SEQUENCE LISTING (3 sequences)

SEQ ID NO: 1 - DNA, 6417 bp, Homo sapiens
aagagtcggcggcggtggcggcggccgctgcagagattggaatccgcctgccgggcttggcgaaggagaagggaggaggcaggagcgaggagggaggagggccaagggcgggcaggaaggcttaggctcggcgcgtccgtccgcgcgcggcgaagatcgcacggcccgatcgaggggcgaccgggtcggggccgctgcacgccaagggcgaaggccgattcgggccccacttcgccccggcggctcgccgcgcccacccgctccgcgccgagggctggaggatgcgttccctggggtccggacttatgaaaatatgcatcagtttaatactgtcttggaattcatgagatggaagcataggtcaaagctgtttggagaaaatcagaagtacagttttatctagccacatcttggaggagtcgtaagaaagcagtgggagttgaagtcattgtcaagtgcttgcgatcttttacaagaaaatctcactgaatgatagtcatttaaattggtgaagtagcaagaccaattattaaaggtgacagtacacaggaaacattacaattgaacaatgcctcagctatacatttacatcagattattgggagcctatttgttcatcatttctcgtgttcaaggacagaatctggatagtatgcttcatggcactgggatgaaatcagactccgaccagaaaaagtcagaaaatggagtaaccttagcaccagaggataccttgccttttttaaagtgctattgctcagggcactgtccagatgatgctattaataacacatgcataactaatggacattgctttgccatcatagaagaagatgaccagggagaaaccacattagcttcagggtgtatgaaatatgaaggatctgattttcagtgcaaagattctccaaaagcccagctacgccggacaatagaatgttgtcggaccaatttatgtaaccagtatttgcaacccacactgccccctgttgtcataggtccgttttttgatggcagcattcgatggctggttttgctcatttctatggctgtctgcataattgctatgatcatcttctccagctgcttttgttacaaacattattgcaagagcatctcaagcagacgtcgttacaatcgtgatttggaacaggatgaagcatttattccagttggagaatcactaaaagaccttattgaccagtcacaaagttctggtagtgggtctggactacctttattggttcagcgaactattgccaaacagattcagatggtccggcaagttggtaaaggccgatatggagaagtatggatgggcaaatggcgtggcgaaaaagtggcggtgaaagtattctttaccactgaagaagccagctggtttcgagaaacagaaatctaccaaactgtgctaatgcgccatgaaaacatacttggtttcatagcggcagacattaaaggtacaggttcctggactcagctctatttgattactgattaccatgaaaatggatctctctatgacttcctgaaatgtgctacactggacaccagagccctgcttaaattggcttattcagctgcctgtggtctgtgccacctgcacacagaaatttatggcacccaaggaaagcccgcaattgctcatcgagacctaaagagcaaaaacatcctcatcaagaaaaatgggagttgctgcattgctgacctgggccttgctgttaaattcaacagtgacacaaatgaagttgatgtgcccttgaataccagggtgggcaccaaacgctacatggctcccgaagtgctggacgaaagcctgaacaaaaaccacttccagccctacatcatggctgacatctacagcttcggcctaatcatttgggagatggctcgtcgttgtatcacaggagggatcgtggaagaataccaattgccatattacaacatggtaccgagtgatccgtcatacgaagatatgcgtgaggttgtgtgtgtcaaacgtttgcggccaattgtgtctaatcggtggaacagtgatgaatgtctacgagcagttttgaagctaatgtcagaatgctgggcccacaatccagcctccagactcacagcattgagaattaagaagacgcttgccaagatggttgaatcccaagatgtaaaaatctgatggttaaaccatcggaggagaaactctagactgcaagaactgtttttacccatggcatgggtggaattagagtggaataaggatgttaacttggttctcagactctttcttcactacgtgttcacaggctgctaatattaaacctttcagtactcttattaggatacaagctgggaacttctaaacacttcattctttatatatggacagctttattttaaatgtggtttttgatgcctttttttaagtgggtttttatgaactgcatcaagacttcaatcctgattagtgtctccagtcaagctctgggtactgaattgcctgttcataaaacggtgctttctgtgaaagccttaagaagataaatgagcgcagcagagatggagaaatagactttgccttttacctgagactttcagttcgtttgtattctacctttgtaaaacagcctatagatgatgatgtgtttgggatactgcttattttatgatagtttgtcctgtgtccttagtgatgtgtgtgtgtctccatgcacatgcacgccgggattcctctgctgccatttgaattagaagaaaataatttatatgcatgcacaggaagatattggtggccggtggttttgtgctttaaaaatgcaatatctgaccaagattcgccaatctcatacaagccatttactttgcaagtgagatagcttccccaccagctttattttttaacatgaaagctgatgccaaggccaaaagaagtttaaagcatctgtaaatttggactgttttccttcaaccaccattttttttgtggttattatttttgtcacggaaagcatcctctccaaagttggagcttctattgccatgaaccatgcttacaaagaaagcacttcttattgaagtgaattcctgcatttgatagcaatgtaagtgcctataaccatgttctatattctttattctcagtaacttttaaaagggaagttatttatattttgtgtataatgtgctttatttgcaaatcacccactcctttacaaccatactttatatatgtacatacattcatactgtagaaaccagctcatgtgtacctcatatcccatccttaagagaagaaatgttataaagtagaactaaatataaattttcagaattaatgcattcaaagtaatatatcaaatccaggactttgttaacttcaggtaaaaacttcattagggtaatatcatctcaattttttcaaatgaaaggattctctaattagaaatttatatgtcagagctgttataaatttatcaactgtcaaatatgttctggacagctaaatcatttgagatttttggttttttgatttctattccctaacttgtgaagacaatgaaaaatcaggcagaaatatttagtatctagtcagtatctgtagctacactgtataactgttcttcaataaaatggttcatattttatagatgccttgttatctcaagaaatctgatttacataaacttatacttctttaatgctttttaaatatttattctgagcaaacaattcatgagtacatcaagtgagatagttttatttgattataacataaaataaatgtgattatatcacatcatcatcaaaaaggtttaaattaaatgggaggaaatcagcatatgtccacccattaccaaaatttgactatcatttaaggttaaaacttacaaatttgtctgcacatcaaatttcacaaatttgaaaattgccttaaccattttgattaataagtttcatctgccataattaaagtctgaagtgttcatcaagataagtaaatttgcatatggataatacccaataacttgttttttcagaatttttcaccatatgtatactgagaaatacaaatattttaatctgcgttgccgtatgatatgattgcacttagaacacccaatttacttaaatcttggtttacttttgacttgataccataatctttaaaatcatttgtcatctttttttttttttttttgagacggagtctcgctctgtcgcccaggctggactgcggactgcagtggcgcaatctcggctcactgcaagctccgcttcccgggttcacgccattctcctgcctcagcctcccgagtagctgggactacaggcgcccgccaccgcgcccggctaattttttgtatttttagtagagacggggtttcaccttgttagccaggatggtctcaatctcctgacctcatgatccacctgcctcggcctcccaaagttcatttgtcatcttaataaaaatataaagacaggcaaagtttattggaaatgttcaaatggtgtgtggaagcaaaaaattacagccagtatatgagaccactattatggttttttaaaattaacttggtctagtaaaagtgatatcaagagttaatcttagaaacttgctcagtaaaaacattttctagtataacatgttctttaaaaagcaaatgctgccgtctttggaatcttaatctaaaaatgtggccgggcgcggtggctcacgcctgtaatcccaacactttgggaggctgaggcgggtggatcacaaggtcaggagttcaagaccagcctggccaacatggtgaaaccccatctctactaaaaataaaaaactcagccaggcgtggtggcgggtgcctgtaatcccagctactcgggaggctgaagcaggagaattgcttaaaatcagaaggtggaggttgcagtgagctgagatcgtgtcactgcactccagcctgggcaaaagagcgaaactccatctcaaataaacaaacaaataaataacaaaaaacaaaaatgttgcattaaacttagttcttgtctctcctttccactcttattcttaaatctgaagctcatcgactaagtgaaatatttaaagaatatgataggccagcaagaagaagtattatgtagtaccatagttagtaaattcgtaaaaccttggaagccattatttggtcccacttgcaatttagtgtttttgaagtgtgtagcttcattcagatagctctttaaataattaaaatataaaagcaaacaacccaaactacctgactataaacaggaaaagttaaccctcaaagagagttcttgtgaattctctttatgctggcaaatagctctaggattaaaggcacattagggtttccttcagtttgtttattctaagcttttactgtgctttttactgaacaagtttctgatgtataaaacttgcatctgatttctttggaaatattttcacaaaagttattttaatcagtatttttacattgcctttccagtgtccagaagtgtttctaaacttagaaagtgacctatagttttttaaaattatgttttcctagaacgtgccaaattttgatttactctaacaatcagtacttttcttcagatgctttgttctgtttagaacaaaaatgcactatagtttttaaagaatcatgcatctttgggttggcccaggatcaaatttgatattgaataatttattccagggcagctttcataaacatacttcatagatgttgttttgaaatgtttctaaatatctaaaatcatttcaacagcagaaatgatttttattttaacaaaagattatgatagcccttgtagtgtttaaaagtggtcatatttattactgactttgagtcaggtgttaaaatagcagtgccacagctcgtctcttgccttagtgtgctgctgtgagagtcacagtggaaactgcagggaggaggtgtgttcctaagaaccaaaatccagcacagcatcctgtgaagccacgtgtaatgatggtcccataaggaaagtatgtgaatatggctcttgtaaaggattaactattgtaattttagcttatgctctgtattctgttttctatggaattatttaagcccttttagtgacctttgtcctggcccatttaaaaactaaaatgtagtatatattgtataaaatggaaatatcattattgcttcattaggggaaactgtacataggcattgaaagaagggtaaaagcaagcagttttatcaggcagttgtaaaacaccaaaaatatagattcgtctttgacgtgtaacacactaaatgtattttgtacagcatctggtttaaaaggtgccttaagagtttaccattacttgctttgttctatatacagattatgtccaatgtatcattttgaagtaaataaccttattttagtata

SEQ ID NO: 2 - DNA, 12068 bp, Homo sapiens
gactcccccctttgtgtctggtctgctcggagccactggaagtgcctcccggagggacgcagggtgtctcgccgcctccctgcccacccccttccccggctaccttcatccgccctcccgccgccccccgccctcggtccgcgacgcccgagttccgtcaggagcccagagctgcgggagaacgaggcggcggcggcggcggcggcggcggcggcggcggcagcagcagcggcttcctcggggggttgtgattcgctcacaggagccattgacgggagaagaggaggctttcttggtggaatttacctcaggcaagatcgagccgcaggaataaaaagcgaggaagggaagggagcgccgccgggaggactagaaggggcagcctctcacacccactccgcctgccgtctcggggagcccggaccggggccgcgaccgcgacccctcccctcccccgctcctacctctcctcagccttcgccagggcctccccaaccctctcacggttgttctgcgaaggcgtggggactgtgagcttgtccatggaggcaggcaccttttttgatccagtcaaggaagaggatttgttgttttcgaaatcagagtgaaggaagcaccgaagcgaaacttaaggaatcctgccttcccggagccgcgggcgatgcgactagggctgccgggcgccgccgccgcccgtccggcttcgtccttcccggcagtcgggaactagttctgaccctcgccccccgaccccggatcgaatccccgccctccgcaccctggatatgttttctcccagacctggatatttttttgatatcgtgaaactacgagggaaataatttgggggatttcttcttggctccctgctttccccacagacatgccttccgtttggagggccgcggcaccccgtccgaggcgaaggaacccccccagccgcgagggagagaaatgaagggaatttctgcagcggcatgaaagctctgcagctaggtcctctcatcagccatttgtcctttcaaactgtattgtgatacgggcaggatcagtccacgggagagaagacgagcctcccggctgtttctccgccggtctacttcccatatttcttttctttgccctcctgattcttggctggcccagggatgacttcctcgctgcagcggccctggcgggtgccctggctaccatggaccatcctgctggtcagcactgcggctgcttcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagacaataatcattgctttggcatcagtctctgtattagctgttttgatagttgccttatgctttggatacagaatgttgacaggagaccgtaaacaaggtcttcacagtatgaacatgatggaggcagcagcatccgaaccctctcttgatctagataatctgaaactgttggagctgattggccgaggtcgatatggagcagtatataaaggctccttggatgagcgtccagttgctgtaaaagtgttttcctttgcaaaccgtcagaattttatcaacgaaaagaacatttacagagtgcctttgatggaacatgacaacattgcccgctttatagttggagatgagagagtcactgcagatggacgcatggaatatttgcttgtgatggagtactatcccaatggatctttatgcaagtatttaagtctccacacaagtgactgggtaagctcttgccgtcttgctcattctgttactagaggactggcttatcttcacacagaattaccacgaggagatcattataaacctgcaatttcccatcgagatttaaacagcagaaatgtcctagtgaaaaatgatggaacctgtgttattagtgactttggactgtccatgaggctgactggaaatagactggtgcgcccaggggaggaagataatgcagccataagcgaggttggcactatcagatatatggcaccagaagtgctagaaggagctgtgaacttgagggactgtgaatcagctttgaaacaagtagacatgtatgctcttggactaatctattgggagatatttatgagatgtacagacctcttcccaggggaatccgtaccagagtaccagatggcttttcagacagaggttggaaaccatcccacttttgaggatatgcaggttctcgtgtctagggaaaaacagagacccaagttcccagaagcctggaaagaaaatagcctggcagtgaggtcactcaaggagacaatcgaagactgttgggaccaggatgcagaggctcggcttactgcacagtgtgctgaggaaaggatggctgaacttatgatgatttgggaaagaaacaaatctgtgagcccaacagtcaatccaatgtctactgctatgcagaatgaacgcaacctgtcacataataggcgtgtgccaaaaattggtccttatccagattattcttcctcctcatacattgaagactctatccatcatactgacagcatcgtgaagaatatttcctctgagcattctatgtccagcacacctttgactataggggaaaaaaaccgaaattcaattaactatgaacgacagcaagcacaagctcgaatccccagccctgaaacaagtgtcaccagcctctccaccaacacaacaaccacaaacaccacaggactcacgccaagtactggcatgactactatatctgagatgccatacccagatgaaacaaatctgcataccacaaatgttgcacagtcaattgggccaacccctgtctgcttacagctgacagaagaagacttggaaaccaacaagctagacccaaaagaagttgataagaacctcaaggaaagctctgatgagaatctcatggagcactctcttaaacagttcagtggcccagacccactgagcagtactagttctagcttgctttacccactcataaaacttgcagtagaagcaactggacagcaggacttcacacagactgcaaatggccaagcatgtttgattcctgatgttctgcctactcagatctatcctctccccaagcagcagaaccttcccaagagacctactagtttgcctttgaacaccaaaaattcaacaaaagagccccggctaaaatttggcagcaagcacaaatcaaacttgaaacaagtcgaaactggagttgccaagatgaatacaatcaatgcagcagaacctcatgtggtgacagtcaccatgaatggtgtggcaggtagaaaccacagtgttaactcccatgctgccacaacccaatatgccaatgggacagtactatctggccaaacaaccaacatagtgacacatagggcccaagaaatgttgcagaatcagtttattggtgaggacacccggctgaatattaattccagtcctgatgagcatgagcctttactgagacgagagcaacaagctggccatgatgaaggtgttctggatcgtcttgtggacaggagggaacggccactagaaggtggccgaactaattccaataacaacaacagcaatccatgttcagaacaagatgttcttgcacagggtgttccaagcacagcagcagatcctgggccatcaaagcccagaagagcacagaggcctaattctctggatctttcagccacaaatgtcctggatggcagcagtatacagataggtgagtcaacacaagatggcaaatcaggatcaggtgaaaagatcaagaaacgtgtgaaaactccctattctcttaagcggtggcgcccctccacctgggtcatctccactgaatcgctggactgtgaagtcaacaataatggcagtaacagggcagttcattccaaatccagcactgctgtttaccttgcagaaggaggcactgctacaaccatggtgtctaaagatataggaatgaactgtctgtgaaatgttttcaagcctatggagtgaaattattttttgcatcatttaaacatgcagaagatgtttaaaaataaaaaaaaaactgctttatcctcctgtcagcaccccctcccacccctgcaacaaagacttgctttaaatagatttcagctatgcagaaaaatttagcttatgcttccatatttttaaattttgttttttaagttttgcacttttgtttagtctcgctaaagttatatttgtctgttatgaccacagagttatatgtgtgtgtatcaaaagtggtctcaaaatatttttttaagaaaaaaagcaaaaacaatgtattgctgataatcagtttggaccagtttcttaaggtcattaaaacagaagcaaattaagacaggtttgactgcagtggtgtctggtatccatgttttatttctgggcacaagctagtttttatgttgatacgttcctgaacatattatcttgttggacatcttttctcttgtgttttgtttgaatgtgcaatagtttataggccacaaataagctttcttgtaagctctcttcctaacagggcacatattcttccataatataaacacttttctgccccatctcccatacttttgaaggtcagttctatgacagtgaattttgcacaggagaagcagctacctgatttcttactttctctctccttatcatggagaatacagaaacattgtctgaaagggctctaaagaaggaactaccaaaacctgacttgaaatgccatttcttttaaccttccaaatcctaaatgtttccttcaaggcatcttaataaacttatttgcttctggttttgggagttcataagagagaatagaacaaaatacaggacatcaaatattagccatttcccattttattttatttttctatgtaggttcatgttccatgttcatttatttaagaaatacatttttattggtaagcttatagagctacacttatggaatttttaagtaggtaaataaatggttaagacaaaatagtgttatagccttcattctctgaataggccatctttgactcataaaattacccttactgtttattataacttcagaagtaatttatagttctgaacctatagtatcttttaccctgttcccaagcaaagactggtgactttatctgaaaatgattcctcttcccatgacctaaaacactgtgaggaaaaatcattcaagtggcatgccaagtccctatgaaggaagggctgctatcaaacctaccttttttgagcaaactgagactaaacttctctcttttcaaaattgtgttatcttccttaatcctattttcataatttttccttttgccagtttttcacattatctttgatatgtgagcaacatttattatttacattagagtataccttttagtaataaaatgacttgaaatcatattatttttaaaagccctttgcttctttcattacttataatctcctctaaaacaacctctgcatgttttttttaaataaagcactttctgtcaaataatggacttttttctaaacagaaattattttctattaatttgcaaactgatgatttcacttttttttacttttttttcgattatcagagtacttagtaaatgttatatagtttagttctaagatagttccagggattaaaaggttaagaggaaaacacaaatcaccaaatttctgatttatgtttttatctcctgaacaatattttctcactcatattcctctatcttatcacttagctaaagacagcctaaatttcccaattttctgtccaaaatatttgtgatttacttgtatataagccttctcatttgccatgtgctgtgatcttacaagttagatgttactatacctaccatttattcagctggattgctgaacacagttctggattcatagaattaagaatatcttgttagtgcccaggatttccacgttttgtgttttattggcccttttctttattcagccccttaatctattttcagtcttctggcacgtaatttttttcacagttattattcttctattaacaaattatttttatctttcctagtacattttcacttagttctcttgcccttaaatatctttcacatgcattttaggatattcttttcaaatatttgtaggacaactttgaatcaaaataaattatgttccttctccaatttgaagcattgaggataaatgaccatttgaggtctaactgatcttttcctgccagaagagttatcttacgttctgctatatttgtatttgggccagttgattgtaggttgtccaacattttttaatattgggaaaattatgataaaatgctttaaaaattaatatgccagattaaaataactgaatagtttactatttcattcaagcatgtttaaaacaaataatttcctttcaccagtttttcttagtaaactcctgaaaaagtaggaaaggtggaaagtatatatcatttttataaattttaaattgtacatcagacttttaaaatctgtaatatacaagcaagcaaaattattttaaatgacttaattgtatgctaatactcatctgataataaatgcttcttaaagttgacatttaactgctatcacaaagttttatatgtagaaaagtggggtccttttgaataaaagatcattcaactaaaaatattaaaatttatttcactggatggtaatgtaaccttaaaagcatcataataggtaaagtctaatattagttcccttaacaaaatcctaactgtataccagaattaggtcactgaaagaacttgatttgaattacgtttagacaaaaatgatttaattgtaaattcttaaaactttctaaatgcataattggcaaaaaaaaaaacccactgttaccagtgtaggaagttacaagaaggcacatactgaatgctgaagtatacatatgctatttctcttaaacctcagagcaaccatatgagcattgtaattaatattcccattttacagatgaggaaactgaagctaagagaagctaagtaatatgcccaaggtccacatctagtaacagacaaagctgggatttcagtctatgtctgcctctctccacatctctttcatccataccacactgcctacatgccatatgacaggatgtgtaatgggctaacgtttatttaaaaagttcaggccaggtgcagtggcttatgcctataatccccgcactttgggaggccgagccgggtggatcatgaggtcagacgttcaacaccagcctggccaagatggtgaaaccccgtctctattaaaaatattttttaaaaattaactgagcgtggtggtgggcgcctgtagtcccagctactcaggaggctaaggcaggagaatctcttgaacccgagaggtggaggttgcagtgagctgagatcgcgccactgcactgcagcctaggcgacagagcaagactgcctcaaaaaaaaaacataaaaattcagtcactatactctggcacaattttcatttgtatatgagccaaaccacataccttaatgatttgggaagttaggcaaatatgagatatgtagacatatctatgctgattgttgcttgagaaataattactaattctagacaaaactcaatcctgtatcttccatcatgaatcttaaaatcatttcacttcactcctaacatttcttacattgcagaactaatggtaaagtaaattttacggaaaaagttaagatagctttgggaacagagacctttccctaaattgattccatagcagatttgggggaattaacaaagaatttcagtctcatcaatcctttgaatccatcttcaaaacttctgcttttaataactttagaaaatttactaatctatagaactaattgagtaggatataggaaggatacaaggatataatgtcctttttataaaagtttagtatagcttctttacatgtatccacttgttccagaaaatgtgcattggttctgaatgtgaaaatatttaaagagagaaaggaacactcaagtaagtgtgggcttcagtgggaattatcacaaaacattggcaagtatttttatttaaattattttcaaatttgacttctacagccaagtggaattggtaggctgtagctgttacactgaaatttctagtctttgtaagtgcctcctgaaagtcatttaaaatggaaaaatatttcaatgagcttttccttttttcatatttatggacatgaatattttattggagatcattaactcctagaatttgagattatatttccatacaacattttataaagttatgttgaacttactacctgttatgtgcaggttattatgtaactattcacagattgcttcatatattgctttatcttcccatctaacttcttaaagttaaaatccggacacacatgttgattatctagaccagtcattctggaaattgtaacactcccacataaaccccaggagactttttcagaatgcaatgtttctaaatgtactgttactggcagtttactctccagcatataaggttgcattttaacttttagattatgaactgtgcaaactttacccaaaactatcttgcatgattccctcctaaatatattccttgattaagtaaatctggcaaatcactgtttgagctagttacataaaatttgttatcaagagaaggcttttctacaagtttccagattaacataaagaaaagagggaatcacagggcatttaagtgcaccttcccattactttccttaaatcacctcatagttaggctgggcgcggtggctcacgcctgtaatcccagcactttgggaggccgagacggtggatcacgaggtcaggggactgagatcatcctggctaacacgatgaaacctcatctctactaaaaatacaaataattagccaggcatggtggcacgcgcctgtagtcccagctactcgggaggcagagacaggagaatcgtttgcacccgggaggcggaggttgttgcagtgagccgagatcgcgccaatgcactccagcctgggctacagagtgagactccatctcaaaaaaaaacaaaaaaaatcacctcatagtttatgtctgacttactccaaacctcagttatctgatttgtagtctgtgtcaggaaagttttcttcatatctattctgtctccctcctctttgattttaaatttttttcttttacccagtaggacaaaaaagagcagttggtcatcatccccaatattcttagtcttcagtatgcttcaggcctctcaatgaacacttaagtctcaattcttcagacaaaattgcttaagctcttctcctcagtcctattttaatgactttatatttaagaatatagaattatattttcttttatattcaaattcattactccagttaagtaatagtttgatagtttgatagaatcgagagttaagatgtttctatttgaaagtggattcaaccatcagaccaccagcaaatcggcacttaatttttgtgttatctaacattttctattgtggaattttatgattttatattctcattagttataactaaaaagccatgcacacagaattgtatatcattttgccattaaaattttttaacatattgcagcaagcttagttttatgattgagccacaaccttttacatattttttgtatgaaatattaaacactaaatgcaagattaactttcaaaagcaaaccctacattaatcaggtattatctatggacatttttgtagaccacttttgaaatacttattattttgcaacatagactggactatacaactttcatttaacttttaggtgactgatttaagttgagtgtgcatatagagaaaaacctagaaatttatctcatggcagatacatttgaaagtacttcagaagaatttatgctgtatattaaaactaggctcaaaataaatctatcgtatctttaaaagtccaattctgttattactgtgatgtttgtagtgttactattaaacattgtgaacatacacatttttaaaacaacttgaaacccattttaaaatctgggtaagagagaaggaatcttcagaacaaaatcacatcattagggtgtccagtttatgattgaatttttaagcaaattactgtatttgaaactacaacttgatttggttttcagttttaaaaggcaacatgtgggttttatccattttatttatacctttagatttcagaaacatcttcatgttttagatgcattctacagacatcatgttacttaaaaactcagggcccctttcatccctttgtacactgaaaaagttcaattgttagcaagtaagcaattagatccagttgaatatttaaagtgtttgttgcacagttcatttaatgtttcatcttatttgactttttcacatagatataatatcagatttcattaattataaaaagttgcccagttctgtaattactgaacagagggaatgactcaactaattggctacatgttgcaacaaatttaggcctttagagttgaagcactgacttaaaacgacttacatttctgttctttggtcaaatgaccatacatgatatgggacaaattgtttcattttgtttgttttttaataagggaacttggtaaagtagttcctgtcagataggattttctcaagagacaatttaacgttataaagccttctaaaagtgaactaaatattttataactttagtaatagcttggatggttttgagaaaataacctgtatttatcacattgtcaaacagaatttttctttgaatcagacaagttcaagctctaaattgatgtgctatatacttaaaatcctaggaagttatctgtaaccagtctcttgtctcaggctcttcaccttgttaccaatcctcgtaagtatgtaaaggaaacatatttttaaagaagcttaacagtaagaaaaaattactaaaagatgcaattcaaagataggtcccagtttaacactgaattgcttgacttctgtggcttttctttttctggccacatttatttatttaagcaatttttgtatgccttgttatttcatttccatagagattatattgtatcagtgtttatgtaagctggaatcatcctcagttttttgctgataatttttcaaataaagatacatggataattgtaaaatacactaactcttagggtgttgtagtagctgaaacatggagatgcgtagctgtcatgctttttctgaatggacaggagaaacataagctacggagtattcacttctgaggatgcttttccggaaaaagaaaggctagaaaatactcgcacttcctcagaaccctctttcttgttaacgggtatcttttgttggtgtgttttgctcttacattacagatagactatcatatatgactttatgaataatttcagttattttgcttttgtataagctgtctgaagccttgctatgctgtataagttgtgtttgatggatcagtgtgagtataaaataaagcaaatcacttttcttttgtattatctatggatgccactatgaaagctgacattaagccactaaagagttttctatgaataagtgtaagtaaatgctttgatatatataaacctaaataaaaagattgtattgatacagagacattggagaaggagattttaaggcagttctttaggtttaaaaaggcttgctgtaaaatggtgcgttattccgtttattaaagatcatattaatgacaa

SEQ ID NO: 3 - DNA, 56 bp, Artificial Sequence
  Description of Artificial Sequence Synthetic oligonucleotide
  modified_base (56)..(56): a, c, t, g, unknown or other
agcagtggtatcaacgcagagtacttttttttttttttttttttttttttttttvn

(19) United States
(12) Patent Application Publication
(10) Pub. No.: US 2022/0186230 A1
(43) Pub. Date: Jun. 16, 2022

(54) MODULATING BONE MORPHOGENIC PROTEIN (BMP) SIGNALING IN THE TREATMENT OF ALZHEIMER'S DISEASE

(71) Applicants: Chan Zuckerberg Biohub, Inc., San Francisco, CA (US); The Board of Trustees of the Leland Stanford Junior University, Stanford, CA (US)

(72) Inventors: Elizabeth Yang Chen, Chicago, IL (US); Felicia Reinitz, Redwood City, CA (US); Jane Antony, San Jose, CA (US); Michael F. Clarke, Menlo Park, CA (US); Robert C. Jones, San Francisco, CA (US)

(21) Appl. No.: 17/548,269

(22) Filed: Dec. 10, 2021

Related U.S. Application Data
(60) Provisional application No. 63/124,644, filed on Dec. 11, 2020.

(51) Int. Cl. C12N 15/113; A61K 31/7088; A61P 25/28

(52) U.S. Cl. CPC C12N 15/1138; A61P 25/28; A61K 31/7088

(57) ABSTRACT

Methods and compositions are provided for the treatment of Alzheimer's Disease (AD) by administering to a patient a therapeutically effective amount of an agent that inhibits signaling mediated by a bone morphogenetic protein type 1A receptor (BMPR-1A) or bone morphogenetic protein type 2 receptor (BMPR-2). Also provided are methods and compositions to increase the rate of neural stem cell self-renewal.

This application claims the priority benefit of U.S. Provisional Patent Application No. 63/124,644, filed Dec. 11, 2020, the entire content& of which are incorporated herein by reference for all purposes.

### SEQUENCE LISTING

This patent disclosure includes a sequence listing in the form of an ASCII text file entitled 103182-1273222-005710US_SL.txt, dated Dec. 10, 2021, which is 24,944 in size. The sequence listing is hereby incorporated herein by reference as part of the disclosure.

### BACKGROUND OF THE INVENTION

Alzheimer's disease (AD) is the most common form of dementia, occurring in 10% of individuals over the age of 65 and affecting an estimated 5.5 million people in the United States (Hebert et al., 2013). Currently there is no treatment to stop, prevent, or reverse AD (Huang and Mucke, 2012). Historically, AD has been understood by its end-stage disease phenotype, characterized clinically by dementia and pathologically by amyloid senile plaques and neurofibrillary tangles (Castellani et al., 2010). These traditional AD pathologies are associated with inflammation, increased reactive oxygen species (ROS) and neurodegeneration during aging (Akiyama et al., 2000; Glass et al., 2010); however, thus far, treatments to prevent or decrease formation of plaques, tangles and inflammation have not significantly improved disease progression or outcomes (Aisen, 2008; Green et al., 2009; Group et al., 2008).

### BRIEF SUMMARY OF THE INVENTION

Disclosed herein is a method for treating a patient having or at risk of developing Alzheimer's Disease (AD), said method comprising inhibiting signaling mediated by BMPR-1A and/or BMPR-2 in neural stem cells or neural progenitor cells. In an aspect inhibiting signaling included inhibiting expression or activity of BMPR-1A and/or BMPR-2. In an aspect the invention provides an inhibitor of BMPR-1A and/or BMPR-2 for use in the treatment of AD.

### BRIEF DESCRIPTION OF THE DRAWINGS

FIG. 1A shows representative 40X confocal images of the SVZ stains for EdU, GFAP and SOX2. 3-4 month old mice underwent intraperitoneal injections every day for 6 days with EdU and the analysis was performed four weeks after. Count of proliferating NPCs, as cells positive for EdU, GFAP, SOX2 and DAPI is shown in the panel on the right (n=3 mice). Data are presented as mean±SEM. FIG. 1B shows results of limiting dilution assays which were performed using single cells derived from neurospheres from 3-4 month old mice. The graph shows the percentage of neurosphere-initiating cells (NIC)±upper and lower estimates converted to percentages from values calculated by ELDA. FIG. 1C shows cytokine levels measured by Luminex array from the SVZ of young 3-4 months mice. No differences have been observed at this age (n=3 mice for each genotype). Data are shown for the relative fluorescence intensity (RFI) presented as mean±SD. FIG. 1D shows the preference index of NOR 24-hour testing. Mice at 3 months of age showed no signs of cognitive impairment in the Tg-SwDI mice with a preference index comparable to that of WT indicating both genotypes had intact object discrimination (P=0.001 for WT and P=0.0099 for Tg-SwDI, n=7-10 mice in each group). Data are presented as mean±SEM. FIG. 1E shows 1X representative photographs of neurospheres grown in 6-well dish after 14 days of culture (left), ELDA graph of limiting dilution assay comparing human fetal neurospheres infected with pHIV-Zsgreen or mutant APP (center).

FIG. 2A shows results of limiting dilution assays which were performed using single cells derived from neurospheres from 1 year old mice. The graph shows the percentage of neurosphere-initiating cells calculated by ELDA. FIG. 2B shows mRNA level of Cdkn2a in the cerebral cortex of young (3-4 months old) and aged (12 months old) mice that were measured by RT-qPCR. Ct values were normalized to β-Actin. (WT=wild-type littermate; Cdkn2a: nYwt=7, nOwt=6, nOTg-SwDI=6). A one-way ANOVA showed significant differences between the groups (P=0.0063 between Aged WT and Aged Tg-SwDI). Data are presented as mean±SD. FIG. 2C shows mRNA levels of Cdkn2a and Bmi1 during WT neurosphere serial passaging in vitro (n=3 mice, mice were aged 3-4 months). Data are presented as mean±SD. FIG. 2D shows Bmi-1 expression levels measured by RT-qPCR in neurospheres formed from the SVZ of WT or Tg-SwDI mice at 3rd passage (mice aged 3-4 months). Data are presented as mean±SD. FIG. 2E shows images of anterior sections that were obtained from 9-12 months old mice, stained and counted for GFAP+ cells in the cortex. Four different images per sections and three sections per mouse were counted (n=4 mice each group). A one-way ANOVA showed significant differences between the groups (P<0.0001 between Aged WT and Tg-SwDI). Data are presented as mean±SD. FIG. 2F shows cytokine levels measured by Luminex array from the SVZ of 1 year old mice. No differences have been observed at this age. (n=3 mice each genotype). Data are presented as mean±SD.

FIG. 3A shows an ELDA graph of limiting dilution assay comparing young SVZ from WT, Tg-SwDI and Tg-SwDI/Cdkn2a−/− mice (left) and a bar graph illustrating the NIC frequencies in SVZ and in the dentate gyrus (right). FIG. 3B shows percentages of total cells with error bars indicating the upper and lower values. Mice were 3 months old when sacrificed; experiment done after 3rd passage of NSPs. FIG. 3C shows schematic illustrations summarizing the role of Bmi1 in ubiquitinating histone H2A at different sites in the genome including the Cdkn2a locus and the role of Usp16 as its natural antagonist, suggesting that Usp16 inhibition could have an effect on neurosphere initiating capacity. FIG. 3D shows results of RT-qPCR of Cdkn2a in the cerebral cortex of old Tg-SwDI mice. mRNA levels were rescued by Usp16 haploinsufficiency (n=3). Ct-values were normalized to β-actin. A one-way ANOVA showed significant differences between the groups (P=0.0365 between WT and Tg-SwDI and P=0.0318 between Tg-SwDI and Tg-SwDI/Usp16+/−). Data are presented as mean±SD. FIG. 3E shows 1X representative photographs of neurospheres grown in 96-well dish after 2 weeks of culture (left). The bar graph shows the NIC frequencies in SVZ as percentages of total cells comparing WT, Tg-SwDI and Tg-SwDI/Usp16+/− mice (right). Mice were 3 months old. FIG. 3F shows an ELDA graph of limiting dilution assay comparing hippocampal cells obtained from the dentate gyrus.

FIG. 4A shows a schematic illustration of the preparation workflow for the single-cell RNA-seq and gene set enrichment analysis (GSEA). Lineage-CD24− NPCs were FACS-sorted from the SVZ of 4 mice each of the different genotypes and processed for single-cell RNA-sequencing. FIG. 4B shows enrichment plots illustrating TGF-β signaling pathway as enriched in Alzheimer's and rescued by Usp16 haploinsufficiency. FIG. 4C shows heatmaps illustrating averaged normalized single-cell gene expression of elements of the TGF-β pathway; elements of the BMP pathway, a sub-pathway of the TGF-β pathway, are specifically enriched in Alzheimer's.

FIG. 5A shows representative 100X images of phospho-Smad 1/5/8 staining in mutant APP-infected human fetal neurospheres compared to Zsgreen controls (left) and quantification of DAPI and phospho-Smad1/5/8 co-stained cells in each group (right). Data are presented as mean±SD. FIG. 5B shows representative 10X images of phospho-Smad1/5/8 staining in neurospheres treated with LDN-193189 for 1 week. FIG. 5C shows bar graphs illustrating the quantification of phospho-SMAD 1/5/8 after treatment with different doses of LDN-193189. A two-way ANOVA revealed significant differences between the groups (**** for P<0.0001). Data are presented as mean±SD. FIG. 5D shows representative 6X images of in vitro colonies of mutant APP- and Zsgreen-infected human fetal neurospheres after 1 week of LDN-193189 treatment. FIG. 5E shows bar graphs illustrating quantification of the colonies in (D). A two-way ANOVA revealed significant differences between groups (**** for P<0.0001 and *** for P=0.0003). Data are presented as mean±SD.

FIG. 6A shows bar graphs illustrating quantification of GFAP+ cells from cortex. A one-way ANOVA showed significant differences between the groups (P=0.0012 between WT and Tg-SwDI and P=0.0188 between Tg-SwDI and Tg-SwDI/Usp16+/−). Data are presented as mean±SD. FIG. 6B shows bar graphs illustrating quantification of area covered by plaques using thioflavin S staining in Tg-SwDI and Tg-SwDI/Usp16+/− mice shows no difference between the two genotypes (10-month-old mice). Data are presented as mean±SEM. FIG. 6C shows bar graphs illustrating the preference index of NOR 24-hour testing in mice at 6 months of age. The earliest signs of cognitive impairment are present in the Tg-SwDI mice with a preference index of 49%, while WT and Tg-SwDI/Usp16+/− mice had preference indexes >65% indicating intact object discrimination (P=0.001 for WT and P=0.0099 for Tg-SwDI/Usp16+/−, n=7-10 mice). Data are presented as mean±SEM. FIG. 6D shows a schematic illustration summarizing the temporal effects of mutant APP demonstrated in the examples above.

FIG. 7A shows cytokine levels measured by Luminex array from the DG. FIG. 7B shows cytokine levels measured by Luminex array from the cortex. Both shows levels for 3-4 months mice. No differences have been observed at this age. N=3 mice for each genotype. Data are presented as mean+/−SD.

FIG. 8A shows Number of times neurospheres were serially passaged before expiring; WT control cells serially passage beyond what is shown and were collected for storage or experimentation prior to expiring. Each dot represents a neurosphere culture derived from an individual mouse SVZ. FIG. 8B shows cytokine levels measured by Luminex array from the DG (top) and the cortex (bottom) of 1 year old mice. No differences have been observed at this age. N=3 mice per genotype. Data are presented as mean+/−SD.

FIG. 9 shows RNA-sequencing workflow in 2 year old mice. GLAST+ NPCs were magnetically enriched from the SVZ of four mice of each genotype.

FIG. 10A shows cytokine levels measured by Luminex array from the SVZ. FIG. 10B shows cytokine levels measured by Luminex array from the dentate gyrus (DG). FIG. 10C shows cytokine levels measured by Luminex array from the cortex. All graphs show cytokine levels for 1 year old mice. No differences have been observed at this age for any of the genotypes. Data are presented as mean+/−SD.

### LIST OF TABLES

Table 1: summarizes the lower, upper and estimates of 1/NIC for the different genotypes calculated by ELDA.

Table 2: Lists the estimated stem cell frequencies and ranges for each group, calculated using the ELDA software (n=3 separate infections and limiting dilution experiments) (P=5.33e-7).

Table 3 summarizes the lower, upper and estimates of 1/NIC for the different genotypes calculated by ELDA.

Table 4. Confidence intervals for 1/NIC in young mice.

Table 5. Pairwise tests for differences in stem cell frequencies.

Table 6: GSEA analysis from single-cell RNA-seq data shows pathways enriched in Tg-SwDI mice compared to WT and rescued in Tg-SwDI/Usp16+/− mice. (n=4 for each genotype at each time point; FDR<25%).

Table 7: Normalized Enrichment Scores of Significantly Enriched Pathways.

Table 8: Pathways Rescued by Usp16 Haploinsufficiency in Tg-SwDI mice.

### DETAILED DESCRIPTION OF THE INVENTION

### 1. Definitions

As used herein, the term “Alzheimer's disease”, also referred to herein as “AD”, means a dementia which is primarily identified by clinical diagnosis and established by markers of the disease. AD is a neurological disorder clinically characterized by the accumulation of amyloid-b (Ab) plaques and neurofibrillary tangles, synaptic and neuronal loss, and/or cognitive decline (Lardenoije et al., Prog Neurobiol, (2015). However, AD pathology begins prior to the onset of clinical symptoms. For example, amyloid plaques, one marker of AD pathology, form 10-20 years prior to the onset of AD dementia. AD progresses along a continuum with three broad phases: preclinical AD, mild cognitive impairment (MCI) due to AD, and dementia due to AD (see e.g., Alzheimer's Association, “Alzheimer's Association Report: 2020 Alzheimer's disease facts and figures,” Alzheimers Dement. 2020; 16(3):391-460). The AD dementia phase is further broken down into the stages of mild, moderate and severe, which reflect the degree to which symptoms interfere with one's ability to carry out everyday activities. The clinical disease stage can be characterized by measures, and changes in these measures over time, such as amyloid-beta accumulation (CSF/PET), synaptic dysfunction (FDG-PET/fMRI), tau-mediated neuronal injury (CSF), brain structure (volumetric MRI), cognition, and clinical function (Clifford Jack et al. Lancet. Neurol. 2010 January; 9(1):119). AD is most common in people over the age of 65 with the risk of AD increasing with age. 4-5% of cases are early-onset AD cases where AD is diagnosed before the age of 65. Often, these patients are in their 40 s or 50 s when they're diagnosed with the disease. People with Down syndrome have a higher risk for early-onset AD. Many individuals with early onset have Familial Alzheimer's disease (FAD) which is often caused by autosomal dominant mutations (e.g., mutations in amyloid precursor protein, presenilin-1, and presenilin-2 genes), afflicting less than 1% of all AD cases.

As used herein, the term “Mild cognitive impairment”, also referred to herein as “MCI” (also known as incipient dementia, or isolated memory impairment) is a diagnosis given to individuals who have cognitive impairments beyond that expected for their age and education, but that typically do not interfere significantly with their daily activities (see, e.g., Petersen et al. (1999) Arch. Neurol. 56(3): 303-308). It is considered in many instances to be a boundary or transitional stage between normal aging and dementia.

The term “agent” refers to any molecule, either naturally occurring or synthetic with the desired property. Agents may be, without limitation, a protein, polypeptide, small molecule, antibody, polysaccharide, lipid, fatty acid, inhibitory RNA (e.g., siRNA or shRNA), polynucleotide, aptamer, affimer, chimeric protein, or inhibitor cysteine-knot.

As used herein, the term “inhibiting signaling” refers to the prevention or reduction of signal transduction by a molecule (e.g., BMPR-1A and/or BMPR-2). For example, inhibiting signaling may affect signal transduction in the pathways upstream or downstream of BMPR-1A and/or BMPR-2. In some embodiments, inhibiting signaling may prevent or reduce the effect of BMPR-1A and/or BMPR-2 on one or more pathways (e.g, SMAD signaling pathway). Inhibiting signaling may be accomplished, for example, by inhibiting expression or activity of the target molecule (e.g., of BMPR-1A and/or BMPR-2). In some embodiments, inhibiting signaling includes inhibiting the expression of the target molecule by nucleic acids, such as siRNA or ASOs. In some embodiments, inhibiting signaling can be achieved by inhibiting the activity of the molecule, e.g., with a small molecule inhibitor. Determining the effect of an inhibitory agent on BMPR-1A and/or BMPR-2 activity can be measured using one or more methods known in the art, including but not limited to, half maximal inhibitory concentration (IC₅₀), dissociation constant (K_{D}), and inhibitor constant (K_{I}). For example, IC₅₀ is a measure of the effectiveness of a substance in inhibiting a specific biological or biochemical function. In some embodiments, inhibition of signaling can be identified by measuring the induction of phosphorylation of downstream targets (e.g., SMADs). For example, inhibition of signaling of BMPR-1A and/or BMPR-2 may be measured by determining levels of phospho-Smad1/5/8.

The term “small molecule inhibitor” as used herein, refers to a molecule (e.g., an organic molecule) having a molecular weight of less than about 10,000, e.g., less than 5,000, less than 2500 Daltons, than 2000, less than 1500, or less than 1000, wherein the molecule is capable of inhibiting, to some measurable extent, the activity of a molecule (e.g., BMPR-1A and/or BMPR-2).

As used herein, the term “inhibition”, or any grammatical variation thereof (e.g., inhibit, inhibiting, etc.) as referred to herein, relates to the retardation, restraining or reduction of the mRNA and/or protein levels, expression and/or activity of a molecule (e.g., BMPR-1A and/or BMPR-2) by at least at least 10%, at least 20%, at least 30%, at least, at least 40%, at least 50%, at least 60%, at least 70%, at least 80%, at least 90%, or 100%, or any percentage in between.

As used herein, the terms “self-renewal” or any grammatical variation thereof (e.g., self-renew, self-renewing, etc.) refers to the capability of a stem cell (e.g., a neural stem cell) to divide to produce two daughter cells, at least one of which is a multipotent stem cell (e.g., a neural stem cell). As such, self-renewal is the process by which stem cells divide to make more stem cells with maintenance of the undifferentiated state. When a stem cell divides symmetrically, both resulting daughter cells are equivalent. For example, a stem cell may undergo a self-renewing symmetric division in which both resulting daughter cells are stem cells with an equal amount of differentiation potential as the mother cell. However, a symmetric division is not necessarily a self-renewing division because both resulting daughter cells may instead be differentiated relative to the mother cell. When a stem cell divides asymmetrically, the resulting daughter cells are different than one another. For example, if a stem cell undergoes a self-renewing asymmetric division, then one of the resulting daughter cells is a stem cell with the same amount of differentiation potential as the mother cell while the other daughter cell is differentiated relative to the mother cell (e.g., a more lineage restricted progenitor cell, a terminally differentiated cell, etc.). A stem cell may directly differentiate (i.e., without dividing), or may instead produce a differentiated cell type through an asymmetric or symmetric cell division.

The term “stem cell” is used herein to refer to a cell that has the ability both to self-renew and to generate a differentiated cell type. A “differentiated cell” is a cell that has progressed further in the developmental pathway than the cell it is being compared with. For example, multipotent stem cells (e.g., neural stem cells) can differentiate into further restricted stem cells (e.g., neural progenitor cells), which in turn can differentiate into cells that are further restricted (e.g., glial-restricted neural progenitor cells), which can differentiate into end-stage cells (i.e., terminally differentiated cells, e.g., neurons), may not retain the capacity to proliferate further. Different types of stem cells may be characterized by both the presence and the absence of specific markers of specific markers (e.g., proteins, RNAs, etc.).

As used herein “neural stem cell” or “NSC” refers to a cell of the central nervous system (CNS) that can self-renew and that has sufficient potency to differentiate into more specialized cell types of the CNS (e.g., neural progenitor cells).

As used herein “neural progenitor cell” or “neuronal precursor cell” refers to a cell that is further differentiated relative to the stem cell that gave rise to it (neural stem cell) and can give rise to cells that are further differentiated (e.g., terminally differentiated cells such as neurons, astrocytes, and oligodendrocytes).

As used herein, the term “nucleic acid” and “polynucleotide” are used interchangeably and refer to a polymer of nucleotides, including deoxyribonucleic acids (DNA), ribonucleic acids (RNA), or any combination and polymers thereof in either single- or double-stranded form. The term encompasses nucleic acids containing modified nucleotides.

The term “protein” are used herein and refer to a polymer of amino acid residues. As used herein, the terms encompass amino acid chains of any length, including full-length proteins and truncated proteins.

As used herein, the term “complementary” refers to specific base pairing between nucleotides or nucleic acids. Complementary nucleotides are, generally, adenine (A) and thymine (T) (or A and uracil (U)), and guanine (G) and cytosine (C). It will be understood that term also encompasses base paring between modified nucleotides, or between non-modified and modified nucleotides.

As used herein, an “antisense polynucleotide”, “antisense oligonucleotide” or “ASO” is a single-stranded nucleic acid sequence (DNA, RNA, or a nucleotide analog) capable of hybridizing to a target RNA sequence (e.g., a BMPR-1A and/or BMPR-2 mRNA). Upon binding to their target RNA, ASOs can inhibit gene expression and/or initiate the degradation of the target RNA through various mechanisms, for example by inducing cleavage of the target RNA through endoribonuclease (RNase) recruitment.

The term “hybridizes” or any grammatical variation thereof (e.g., hybridizing, hybridization, etc.) and “bind” or any grammatical variation thereof (e.g., binding, etc.) are used interchangeably and refer to the annealing of two nucleic acids strands. In particular, two nucleic acid strands form hydrogen bonds between base pairs of the two strands, thereby forming a duplex. In certain embodiments, an antisense oligonucleotide, an siRNA, or a shRNA may hybridize with a target nucleic acid sequence contained in mRNA encoding BMPR-1A or BMPR-2.

The terms “identical” or percent “identity,” in the context of two or more nucleic acids or polypeptide sequences, refer to two or more sequences or subsequences that are the same (“identical”) or have a specified percentage of amino acid residues or nucleotides that are the same (i.e., at least about 70% identity, at least about 75% identity, at least 80% identity, at least about 90% identity, preferably at least about 91%, 92%, 93%, 94%, 95%, 96%, 97%, 98%, 99%, or higher identity over the entire sequence of a specified region, when compared and aligned for maximum correspondence over a comparison window or designated region. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). Algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information.

As used herein, the term “small interfering RNA (siRNA)” refers to a double-stranded RNA (or RNA analog) that is capable of directing or mediating RNA interference. In some embodiments, the siRNA is 10-50 nucleotides (or nucleotide analogs), e.g., 12-30 nucleotides in length, e.g., 15-25 nucleotides in length, e.g., 19-23 nucleotides in length, e.g., 21-23 nucleotides in length.

The term “short hairpin RNA”, “small hairpin RNA”, and “shRNA” are used interchangeably and refer to a double-stranded interfering RNA (e.g., siRNA) where the two strands are connected to form a hairpin or loop region.

The term “guide RNA” or “gRNA”, as used herein refers to a nucleic acid that binds to a Cas protein and aids in targeting the Cas protein to a specific target sequence within DNA. A gRNA may comprise a crisp RNA (crRNA) and a transactivating crisp RNA (tracrRNA).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term includes antibody fragments having the same antigen specificity, and fusion products thereof.

An exemplary immunoglobulin (antibody) structural unit comprises a tetramer. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. Thus, the terms “variable heavy chain,” “V_{H}”, or “VH” refer to the variable region of an immunoglobulin heavy chain, including an Fv, scFv, dsFv or Fab; while the terms “variable light chain,” “V_{L}”, or “VL” refer to the variable region of an immunoglobulin light chain, including of an Fv, scFv, dsFv or Fab. Equivalent molecules include antigen binding proteins having the desired antigen specificity, derived, for example, by modifying an antibody fragment or by selection from a phage display library.

The term “treatment” or any grammatical variation thereof (e.g., treat, treating, etc.), refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, decreasing the rate of disease progression, amelioration or palliation of the disease state, diminishment of any direct or indirect pathological consequences of the disease, improved prognosis, or preventing or decreasing cognitive decline.

The term “subject” or “patient” refers to a human or an animal (particularly a mammal) that receive either prophylactic or therapeutic treatment. For example, a subject can be a human.

“Pharmaceutically acceptable carrier” and “pharmaceutically acceptable excipient” are used interchangeably and refer to a substance or compound that aids or facilitates preparation, storage, administration, delivery, effectiveness, absorption by a subject, or any other feature of the composition for its intended use or purpose. Such a pharmaceutically acceptable carrier is not biologically or otherwise undesirable and can be included in the compositions of the present invention without causing a significant adverse toxicological effect on the subject or interacting in a deleterious manner with the other components of the pharmaceutical composition.

As used herein, the term “administering”, “administration”, or “administer” means delivering the pharmaceutical composition as described herein to a target cell or a subject. The pharmaceutical compositions described herein are designed for delivery to subjects in need thereof by any suitable route or a combination of different routes. In particular embodiments, pharmaceutical compositions are administered by intravenous injection or oral administration.

### 2. Introduction

Adult neurogenesis is thought to be compromised in Alzheimer's disease (AD), contributing to early dementia (Alipour et al., 2019). The decline of neural stem/progenitor cell (NPC) function in the subventricular zone (SVZ) and the hippocampus has been established in both aging (Leeman et al., 2018) and various AD mouse models (Haughey et al., 2002; Lopez-Toledano and Shelanski, 2004; Mu and Gage, 2011; Rodriguez et al., 2009; Rodriguez and Verkhratsky, 2011; Sakamoto et al., 2014; Winner et al., 2011). Current strategies to reverse neurogenesis defects include the use of drugs (“senolytics”) that selectively remove p16Ink4a-positive senescent cells. Removal of p16Ink4a-positive senescent cells, for instance, using a suicide gene under the regulation of the Cdkn2a promoter has been shown to attenuate progression of age-related decline and preserve cognitive function in both an accelerated aging AD mouse model and a tauopathy mouse model (Baker et al., 2011; Bussian et al., 2018). However, the use of a suicide gene is not directly translatable into humans, and other senolytics such as BCL2-inhibitors or the combination of Desatanib and quercetin have toxicities which can limit their use (Amaya-Montoya et al., 2020; Zhu et al., 2015). Additionally, in the brain, it is not clear whether it is more effective to remove the p16Ink4a-positive glial cells after their development or prevent their development altogether as clearance of these cells does not reverse aging (Baker et al., 2011).

Here we investigate whether neurogenesis defects are cell-intrinsic, resulting from changes inside the cells, or extrinsic as a result of external niche factors such as inflammation. We have determined, based on work described in the examples in an AD mouse model harboring Swedish, Dutch, and Iowa mutations in the amyloid precursor protein (Tg-SwDI), that cell intrinsic neural precursor cell defects precede inflammation, an extrinsic factor. In addition, we show that this defect is partly regulated by Cdkn2a, a central component of aging and decreased neurogenesis of NPCs and differentiated cells. As inhibiting Cdkn2a can result in tumor formation, we explored modulation of its upstream regulator, USP16. When we inhibited USP16 by inducing USP16 haploinsufficiency in Tg-SwDI mice (Tg-SwDI/Usp16+/−), we found a rescue in the self-renewal of NPCs as early as 3 months of age. USP16 haploinsufficiency also decreased astrogliosis and cognitive decline. As Usp16 is a deubiquitinating enzyme and may have global epigenetic effects, targeting one of its downstream pathways might limit off-target effects. We thus proceeded to broaden our perspective to the specific pathway(s) Usp16 was affecting through single-cell RNA-sequencing of neural stem cells isolated from all three genotypes followed by pathway analysis. Our single-cell RNA-sequencing analysis revealed the BMP pathway enriched early on in AD and rescued with Usp16 haploinsufficiency. Analysis of key genes involved in the BMP pathway led us to discover that our neural precursor cells highly express bone morphogenetic protein receptors (BMPRs), in particular BMPR-2 and BMPR-1A. To functionally test whether BMP signaling enrichment could play a role in the stem cell defect present in the human AD model, we used a BMP receptor inhibitor and discovered that BMPR inhibition rescues mutant APP mediated self-renewal defects in human neurospheres.

Thus, in one aspect, the present disclosure provides methods and compositions for treating a patient having a neurodegenerative disease, such as AD, by administering to the patient a therapeutic amount of an agent that inhibits signaling mediated by BMPR-1A and/or BMPR-2. In some aspects, such an agent may antagonize the expression or activity of BMPR-1A and/or BMPR-2. In another aspect, the present disclosure provides methods of increasing the rate of neural stem cell self-renewal in a patient having AD, the method comprising administering to the patient a therapeutically effective amount of an agent that inhibits signaling of BMPR-1A and/or BMPR-2.

### 2.1 Bone Morphogenetic Protein (BMP) Pathway

The BMP pathway is part of the larger family of Transforming Growth Factor β TGF-β signaling pathway. TGF-β signaling involves binding of a ligand to a type II receptor, which recruits and phosphorylates a type I receptor. The type I receptor then phosphorylates a regulatory SMAD (R-SMAD), either SMAD1/5/8 in the case of the BMP pathway, or SMAD2/3 in the case of the TGF-β or the Activin pathway. At this point, the pathways converge when the phosphorylated RSMADs recruit co-SMAD, SMAD4, which helps the entire SMAD complex translocate into the nucleus and activate certain context-dependent genes related to proliferation, differentiation, or other cellular processes68. Activation of the BMP pathway specifically induces expression of Id genes such as ID1, ID2, ID3, and ID4.

The TGF superfamily of ligands includes two major branches, characterized by TGF-β/activin/nodal and Bone Morphogenetic Proteins (BMPs). Both, the type I and the type II receptors have a short extracellular domain, a single transmembrane domain, and an intracellular domain with serine/threonine kinase activity. There are a total of seven type I receptors (ALK1-7) for the TGF-β family of ligands, three of which bind BMPs: type 1A BMP receptor (BMPR-1A or ALK3), type 1B BMP receptor (BMPR-1B or ALK6), and type 1A activin receptor (ActR-1A or ALK2). There are at least four type II receptors for the TGF-β family, three of which are known to interact with BMPs: type 2 BMP receptor (BMPR-2), type 2 activin receptor (ActR-2A or ACVR2A), and type 2B activin receptor (ActR-2B or ActR2b).

### 3. Therapeutic Agents

### 3.1 Agents that Bind a BMPR-1A and/or BMPR-2 Protein and/or Inhibit Interaction Between BMPR-1A and/or BMPR-2 Protein and its Ligand

In some embodiments, the patient is administered an agent that inhibits signaling mediated by BMPR-1A and/or a BMPR-2 by binding a BMPR-1A and/or a BMPR-2 protein. In some embodiments, the agent binds BMPR-2 at a site that is a binding site between BMPR-2 and a ligand (e.g., BMP2) to inhibit the activity of BMPR-2. In some embodiments, the agent binds to BMPR-2 at a site other than a binding site between BMPR-2 and a BMP (e.g., BMP2).

In some embodiments, the patient is administered an agent that inhibits interaction between a BMPR-1A and/or BMPR-2 protein and a BMP. In some embodiments, the agent inhibits interaction between BMPR-1A and/or BMPR-2 and a BMP (e.g., BMP-7, BMP-2, BMP-4). In some embodiments, the agent binds to BMPR-1A and/or BMPR-2 at or near its binding site for a BMP, thus inhibiting the ability of BMPR-1A and/or BMPR-2 to bind to the BMP. In some embodiments, an agent that inhibits interaction between a BMPR-1A and/or BMPR-2 protein and a BMP inhibits the signaling pathway that is initiated by binding of BMPR-1A and/or BMPR-2 to the BMP.

In some embodiments, the patient is administered an agent that inhibits complex formation between a BMPR-1A protein and BMPR-2 protein. In some embodiments, the agent binds to BMPR-1A at or near a site that inhibiting the ability of BMPR-1A to interact and build a complex with BMPR-2. In some embodiments, an agent that inhibits complex formation between a BMPR-1A protein and BMPR-2 protein inhibits the signaling pathway that is initiated by complex formation of a BMPR-1A protein and a BMPR-2 protein.

The sections that follow describe different classes of inhibitors of BMPR-1A and/or BMPR-2 binding proteins and antagonists. The development of small molecule inhibitors, antibodies, aptamers, affimers, and other inhibitory agents is generally done by designing or screening a test compound, and then confirming or further selecting candidates with sufficient potency and specificity in one or more suitable assays.

Binding to BMPR-1A and/or BMPR-2 can be determined, for example, by labeling the inhibitor, and determining whether the label is captured using an antibody to the BMPR-1A and/or BMPR-2 protein. Ability to block binding to binding to BMP can be determined, for example, by obtaining a system that quantifies the degree of binding (for example, by fixing BMPR-1A and/or BMPR-2 on a solid surface or cell and labeling the conjugate binding partner, or by measuring change in apparent molecular weight of BMPR-1A and/or BMPR-2 in a Western blot). The test compound is then added to the system, and its ability to inhibit the binding reaction can be quantified relative to control. Other activities normally ascribed to BMPR-1A and/or BMPR-2, such as the triggering of Nodal signaling, can be measured in cultured cells that are capable of demonstrating the activity in question. BMPR-1A and/or BMPR-2 is then combined with or expressed by the cells, in the presence and absence of the test compound. Effective inhibition is measurable by a decrease in the activity normally attributed to or caused by BMPR-1A and/or BMPR-2. With a view to development of an inhibitor as a therapeutic agent, drug candidates that show promise in a binding or cellular assay are then tested in a suitable preclinical model for the intended indication.

### 3.1.1 Small Molecule Inhibitors

In one approach, methods for treating dementia include targeting the BMPR protein using a small molecule inhibitor of BMPR activity. Small molecule inhibitor of BMPR activity include naturally occurring and synthetic small molecule compounds. Naturally occurring or synthetic small molecule compounds of interest include numerous chemical classes, such as organic molecules, e.g., small organic compounds having a molecular weight of more than 50 and less than about 10,000, optionally less than 5,000, sometimes less than 2,500 Daltons. In some embodiments, the small molecule inhibitor has a molecular weight of less than about 1000 Da, or less than about 500 Da. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

In some embodiments, the small molecule inhibitor compound inhibits BMPR-1A (or ALK3) activity. In some embodiments, the small molecule inhibitor compound inhibits BPMR-2 activity. The small molecule inhibitor of BMPR-1A may bind to the ATP binding site of BMPR-1A covalently or non-covalently to inhibit its activity. In other embodiments, the small molecule inhibitor may bind to other parts of BMPR-1A outside of the ATP binding site. For example, the small molecule inhibitor may form a covalent interaction with an amino acid (e.g., methionine, tyrosine, or serine) outside of the ATP binding site to inhibit BMPR-1A activity. In some embodiments, a small molecule inhibitor may also bind to BMPR-1A to cause a conformational change in BMPR-1A that prevents BMPR-1A from functioning. In some embodiments, the small molecule inhibitor may bind to BMPR-1A with a higher affinity than to ActR-1A (or ALK2). In some embodiments, a small molecule inhibitor may bind to an amino acid or a portion of BMPR-1A, that is different from the corresponding amino acid or portion of ActR-1A, to achieve selective inhibition of BMPR-1A over ActR-1A.

Examples of small molecule inhibitors of BMPR-1A and BMPR-2 are described in e.g., WO Patent Publications WO2009/114180, WO2012/100229, WO2014/160203, WO2016/011019, WO2014/051698, WO2014/138088, and Hopkins (2016), “Inhibitors of the bone morphogenetic protein (BMP) signaling pathway: a patent review (2008-2015),” Expert Opin. Ther. Pat. 26 (10), 1115-1128; Lowery et al. (2016), “A survey of strategies to modulate the bone morphogenetic protein signaling pathway: current and future perspectives,” Stem Cells Int.; 2016:7290686, all of which are incorporated herein by reference for teaching of the small molecule inhibitors. In some embodiments, the small molecule inhibitor is LDN193189 (or DM3189; CAS No. 1062368-24-4). See e.g., WO Patent Publications WO2009/114180, WO2012/100229, all of which are incorporated herein by reference for teaching of the LDN193189 small molecule inhibitor. LDN193189 is also commercially available, e.g., from Selleckchem (Catalog No. 52618) or Sigma-Aldrich (Catalog No. SML0559). In some embodiments, the small molecule compound inhibits BMPR-1A to a greater extent than ActR-1A. Such small molecule inhibitors include, for example, compound 6 described in WO Patent Publication WO2014/160203, compound 63 described in WO Patent Publication WO2014/051698, and compounds 26, 33, 27, 35 and 32 described in WO Patent Publication WO2016/011019. These compounds can be prepared by processes known to the skilled person (see, for example, Surmacz et al., Stem Cells 2012; 30: 1875-1884).

Additional small molecule inhibitors can be identified using known methods in the art, for example, by rational drug design or screening. Approaches for drug design and suitable screening methods are described e.g., in Janzen (2014), “Screening technologies for small molecule discovery: The state of the art,” Chemistry and Biology, 21 (9), 1162-1170; Imming et al. (2006), “Drugs, their targets and the nature and number of drug targets,” Nature Reviews. Drug Discovery, 5 (10), 821-34; and Anderson (2003), “The process of structure-based drug design,” Chemistry and Biology, 10 (9), 787-97.

Screening for candidate agents should identify agents that inhibit BMPR-1A and/or BMPR-2 signaling. In some cases, a BMPR-1A and/or BMPR-2 small molecule inhibitor can be identified by its ability to bind to the BMPR-1A and/or BMPR-2 protein, and fully inhibit the activity of BMPR-1A and/or BMPR-2. In some cases, candidate agents may be screened for increasing the rate of stem cell renewal. For example, in screening assays for biologically active agents, cells expressing BMPR-1A and/or BMPR-2 (e.g., neural stem cells) are contacted with a candidate agent of interest and the effect of the candidate agent on the cell is assessed by monitoring one or more output parameters. Cells useful for screening include cells that express BMPR-1A and/or BMPR-2 (e.g., a neural stem cell). Determining the effect of the agent on BMPR-1A and/or BPMR-2 can be measured using one or more methods known in the art, including but not limited to, half maximal inhibitory concentration (IC₅₀), dissociation constant (K_{D}), and inhibitor constant (K_{I}). For example, IC₅₀ is a measure of the effectiveness of a substance in inhibiting a specific biological or biochemical function. This value indicates the concentration of the substance needed to inhibit a given biological process (or component of the biological process) by half.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. See also e.g., “The Pharmacological Basis of Therapeutics,” Goodman and Gilman, McGraw-Hill, New York, N.Y., (2018), 13th edition.

### 3.1.2 Antibodies

In some embodiments, the agent is an anti-BMPR-1A or anti-BMPR-2 antibody or an antigen-binding fragment thereof. In some embodiments, the antibody is a blocking antibody (i.e., an antibody that binds to a target and directly interferes with the target's function). In some embodiments, the antibody is a neutralizing antibody (i.e., an antibody that binds to a target and negates the downstream cellular effects of the target. In some embodiments, the antibody binds to BMPR-1A, e.g., human BMPR-1A. In some embodiments, the antibody binds to BMPR-2, e.g., human BMPR-2.

In some embodiments, the antibody is a monoclonal antibody. In some embodiments, the antibody is a polyclonal antibody. In some embodiments, the antibody is a chimeric antibody. In some embodiments, the antibody is a humanized antibody. In some embodiments, the antibody is a human antibody. In some embodiments, the antibody is an antigen-binding fragment, such as a F(ab′)2, Fab′, Fab, scFv, and the like. The term “antibody or antigen-binding fragment” can also encompass multi-specific and hybrid antibodies, with dual or multiple antigen or epitope specificities.

In some embodiments, the agent is an anti-BMPR-1A antibody. Anti-BMPR-1A antibodies are commercially available, e.g., from Abcam and Invitrogen. Exemplary anti-BMPR-1A antibody includes, but is not limited to, rabbit anti-human BMPR-1A polyclonal antibody (Abcam, Catalog Nos. ab254043 and ab174815; Invitrogen Catalog No 38-6000), and mouse anti-human BMPR-1A monoclonal antibody (Invitrogen Catalog No MA5-17036). In some embodiments, the agent is an anti-BMPR-2 antibody. Anti-BMPR-2 antibodies are commercially available, e.g., from Abcam and BD Biosciences. Exemplary anti-BMPR-2 antibody includes, but is not limited to, mouse anti-human BMPR-2 polyclonal antibody (Abcam, Catalog No. ab14933), mouse anti-human BMPR-2 monoclonal antibody (BD Biosciences, Catalog No.: 612292).

For preparing an antibody that binds to BMPR-1A and/or BMPR-2, many techniques known in the art can be used. See, e.g., Kohler & Milstein, Nature 256:495-497 (1975); Kozbor et al., Immunology Today 4: 72 (1983); Coligan, Current Protocols in Immunology (1991); Harlow & Lane, Antibodies, A Laboratory Manual (1988); and Goding, Monoclonal Antibodies: Principles and Practice (2^{nd} ed. 1986)). In some embodiments, antibodies are prepared by immunizing an animal or animals (such as mice, rabbits, or rats) with an antigen for the induction of an antibody response. In some embodiments, the antigen is administered in conjugation with an adjuvant (e.g., Freund's adjuvant). In some embodiments, after the initial immunization, one or more subsequent booster injections of the antigen can be administered to improve antibody production. Following immunization, antigen-specific B cells are harvested, e.g., from the spleen and/or lymphoid tissue. For generating monoclonal antibodies, the B cells are fused with myeloma cells, which are subsequently screened for antigen specificity.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Additionally, phage or yeast display technology can be used to identify antibodies and heteromeric Fab fragments that specifically bind to selected antigens (see, e.g., McCafferty et al., Nature 348:552-554 (1990); Marks et al., Biotechnology 10:779-783 (1992); Lou et al. m PEDS 23:311 (2010); and Chao et al., Nature Protocols, 1:755-768 (2006)). Alternatively, antibodies and antibody sequences may be isolated and/or identified using a yeast-based antibody presentation system, such as that disclosed in, e.g., Xu et al., Protein Eng Des Sel, 2013, 26:663-670; WO 2009/036379; WO 2010/105256; and WO 2012/009568. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. Nos. 4,946,778, 4,816,567) can also be adapted to produce antibodies. Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or antibodies covalently bound to immunotoxins (see, e.g., U.S. Pat. No. 4,676,980, WO 91/00360; and WO 92/200373).

Antibodies can be produced using any number of expression systems, including prokaryotic and eukaryotic expression systems. In some embodiments, the expression system is a mammalian cell, such as a hybridoma, or a CHO cell. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or be under the control of different promoters. In other embodiments, the V_{H} and V_{L} region may be expressed using separate vectors.

In some embodiments, an antibody comprises one or more CDR, heavy chain, and/or light chain sequences that are affinity matured. Methods for making affinity matured antibodies are known in the art. For example, in some embodiments, phage libraries containing changes in hypervariable regions may be generated to improve the affinity of an antibody. Phage selections may be performed to enrich for clones with high binding affinity. Selected clones may be subsequently sequenced and their binding affinities may be evaluated.

For chimeric antibodies, methods of making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, the antibody comprises one or more CDR, heavy chain, and/or light chain sequences that are humanized. For humanized antibodies, methods of making humanized antibodies are known in the art. See, e.g., U.S. Pat. No. 8,095,890. Generally, a humanized antibody has one or more amino acid residues introduced into it from a source which is non-human. In some embodiments, humanized antibodies comprise one or more variable regions (such as one or more CDRs) or portions thereof that are non-human (e.g., mouse) and one or more constant regions that are derived from human antibody sequences. In some embodiments, humanized antibodies may also contain one or more framework regions or portions thereof that are non-human.

One exemplary approach to antibody humanization is CDR grafting, in which CDR loops comprising the antigen-binding site are grafted onto corresponding human framework regions. Optionally, a computer modeling method can be used to randomize certain framework residues in addition to the CDR grafting. The grafted CDRs and the randomized framework residues are cloned into a phage display library. The phage display library may be screened to identify the clones with the highest binding affinity. As another exemplary approach to antibody humanization, chain shuffling can be performed. In general, chain shuffling involves the construction and screening of two chimeric phage display libraries. For example, a light chain of a non-human antibody (e.g., rodent antibody) is replaced with a light chain from a human antibody library. The resulting hybrid library is screened by panning against the antigen of interest and hybrid antibodies of interest are selected. Next, the heavy chain of the selected hybrid antibodies is replaced with a heavy chain from a human antibody library. The resulting secondary chimeric library is screened to identify humanized antibodies of interest.

In some embodiments, humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody.

As an alternative to humanization, human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

In some embodiments, antibody fragments (such as a Fab, a Fab′, a F(ab′)₂, a scFv, or a diabody) are generated. Various techniques have been developed for the production of antibody fragments, such as proteolytic digestion of intact antibodies (see, e.g., Morimoto et al., J. Biochem. Biophys. Meth., 24:107-117 (1992); and Brennan et al., Science, 229:81 (1985)) and the use of recombinant host cells to produce the fragments. For example, antibody fragments can be isolated from antibody phage libraries. Alternatively, Fab′-SH fragments can be directly recovered from E. coli cells and chemically coupled to form F(ab′)₂ fragments (see, e.g., Carter et al., BioTechnology, 10:163-167 (1992)). According to another approach, F(ab′)₂ fragments can be isolated directly from recombinant host cell culture. Other techniques for the production of antibody fragments will be apparent to those skilled in the art.

Methods for measuring binding affinity and binding kinetics are known in the art. These methods include, but are not limited to, solid-phase binding assays (e.g., ELISA assay), immunoprecipitation, surface plasmon resonance (e.g., Biacore™ (GE Healthcare, Piscataway, N.J.)), kinetic exclusion assays (e.g.,) KinExA®, flow cytometry, fluorescence-activated cell sorting (FACS), BioLayer interferometry (e.g., Octet™ (FortéBio, Inc., Menlo Park, Calif.)), and western blot analysis.

### 3.1.3 Aptamers, Affimers and Knottins

In some embodiments, the agent is a peptide or nucleic acid aptamer. Aptamers are oligonucleotide or peptide molecules that bind tightly to a specific molecular target, such as small molecules, proteins, nucleic acids, and cells. Nucleic acid aptamers are strands of oligonucleotides that can be DNA, RNA, or nucleic acid analogous (XNA). Typically, nucleic acid aptamers are engineered through repeated rounds of in vitro selection (e.g., SELEX (Systematic Evolution of Ligands by Exponential Enrichment) described in Tuerk and Gold (Science (1990) 249:505-510). See also, Jayasena et al., Clinical Chemistry, 1999, 45:1628-1650. Peptide aptamers are artificial proteins that are selected or engineered to bind to specific target molecules, and typically include one or more peptide loops of variable sequence displayed by the protein scaffold. Peptide aptamer selection can be made using different systems, including the yeast two-hybrid system or using combinatorial peptide libraries constructed by phage display and other surface display technologies such as mRNA display, ribosome display, bacterial display and yeast display. See, e.g., Reverdatto et al., 2015, Curr. Top. Med. Chem. 15:1082-1101.

In some embodiments, the agent is an affimer. Affimers are small, highly stable proteins, typically having a molecular weight of about 12-14 kDa, that bind their target molecules with specificity and affinity similar to that of antibodies. Generally, an affimer displays two peptide loops and an N-terminal sequence that can be randomized to bind different target proteins with high affinity and specificity in a similar manner to monoclonal antibodies. Stabilization of the two peptide loops by the protein scaffold constrains the possible conformations that the peptides can take, which increases the binding affinity and specificity compared to libraries of free peptides. Affimers and methods of making affimers are described in the art. See, e.g., Tiede et al., eLife, 2017, 6:e24903. Affimers are also commercially available, e.g., from Avacta Life Sciences.

In some embodiments, the agent is an inhibitor cysteine knot, also referred to as “knottin.” An inhibitor cysteine knot is a protein structural motif that contains three disulfide bridges. A knot is formed by a core of beta strands and disulfide bonds in which two disulfide bonds form a loop through which a third disulfide bond passes. New binding epitopes can be introduced into natural inhibitor cysteine knots using protein engineering. One approach to the production of inhibitor cysteine knots is to create and screen knottin libraries using yeast surface display and fluorescence-activated cell sorting. Methods of engineering inhibitor cysteine knots are described in the art. See, e.g., Kintzing and Cochran, Curr. Opin. Chem. Biol. 34:143-150, 2016.

### 3.2 Agents that Inhibit Expression of BMPR-1A and/or BMPR-2

Methods of treating AD (and other neurodegenerative diseases) in a subject as described herein may be accomplished by administering an agent that inhibits signaling mediated by BMPR-1A and/or a BMPR-2 by inhibiting expression of BMPR-1A and/or a BMPR-2. In some embodiments, the patient is administered a nucleic acid to the subject to decrease or inhibit the expression of the BMPR-1A and/or BMPR-2 gene. In some embodiments, the polynucleotide may be, for example, a DNA oligonucleotide or an RNA oligonucleotide. In other embodiments, the oligonucleotide may be used in a CRISPR/Cas system. An oligonucleotide that inhibits or decreases the expression of the BMPR-1A or BMPR-2 gene may knock out or knock down the BMPR-1A or BMPR-2 gene in the subject.

In some embodiments, the oligonucleotide may be an siRNA or shRNA or antisense RNA. In some embodiments, the oligonucleotide may be an antisense oligonucleotide that mediates an RNase H-dependent cleavage of the mRNA transcript of the BMPR-1A or BMPR-2 gene. In other embodiments, the oligonucleotide may be an miRNA. In yet other embodiments, the oligonucleotide may be used in a CRISPR/Cas system.

In some embodiments, the mRNA transcript of the BMPR-1A or BMPR-2 gene may be targeted for cleavage and degradation. Different portions of the mRNA transcript may be targeted to decrease or inhibit the expression of the BMPR1 gene. In some embodiments, a DNA oligonucleotide may be used to target the mRNA transcript and form a DNA:RNA duplex with the mRNA transcript. The duplex may then be recognized and the mRNA cleaved by specific proteins in the cell. In other embodiments, an RNA oligonucleotide may be used to target the mRNA transcript of the BMPR-1A or BMPR-2 gene.

In some cases, the delivery of the inhibitory polynucleotide may result in a knockdown of BMPR-1A and/or BMPR-2 at least about 25%, 50%, 75%, 80%, 85%, 90%, 95%, 96%, 97%, 98%, 99%, or 100%. In some embodiments, the expression of inhibitory polynucleotide preferentially leads to knockdown of BMPR-1A and/or BMPR-2 compared to other BMPRs (e.g., ACTR-1A).

In some embodiments, the inhibitory polynucleotide targets a sequence that is identical or substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a target sequence in a BMPR-1A or BMPR-2 polynucleotide (e.g., a portion comprising at least at least, 15, at least 20, at least 30, at least 40, at least 50, at least 60, at least 70, at least 80, at least 90, or at least 100 contiguous nucleotides, e.g., from 15-500, 20-250, 20-100, 50-500, or 50-250 contiguous nucleotides of the human BMPR-1A polynucleotide (cDNA) sequence set forth in NCBI Ref. Seq NM 004329.3. (SEQ ID NO: 1) or the human BMPR-2 polynucleotide (cDNA) sequence set forth in NCBI Ref. Seq NM_001204.7 (SEQ ID NO: 2).

In some embodiments, a nucleic acid that targets a BMPR-1A and/or BMPR-2 mRNA and no other BMPR mRNAs can be designed. In one embodiment, target-specific knockdown of BMPR-1A and/or BMPR-2 can be accomplished by designing a nucleic acid (e.g., siRNA or ASO) comprising a sequence that is identical or substantially identical to a region of the BMPR-1A and/or BMPR-2 gene and has low or no homology to the sequence of any other BMPR gene (e.g., gene encoding ACTR-1A). For example, to make siRNAs that preferentially target BMPR-1A and/or BMPR-2 mRNA one would identify a unique region of the BMPR-1A and/or BMPR-2 gene, a region that does not have significant homology to a gene encoding another BMPRs (e.g., ACTR-1A). The specificity or knockdown level of nucleic acid can be confirmed using real-time PCR analysis for mRNA level or ELISA assay for the protein level. To determine if a nucleic acid (e.g., siRNA or ASO agent) preferentially targets BMPR-1A and/or BMPR-2 mRNA over other BMPR mRNAs one can transfect or transduce the polynucleotide tagged to marker such as GFP in a cell line or other expression system, select the GFP positive cells (e.g. transformed cells), and determine the level of BMPR-1A and/or BMPR-2 knockdown relative to expression of other BMPRs (e.g., ACTR-1A) in the cell system without transfection or transduction with the polynucleotide agent. In some embodiments, the expression of RNA is measured. In other embodiments, the expression of the protein is measured. In one example, mRNA may be measured by any PCR-based assay known in the art (e.g., RT-PCR or qRT-PCR or the like). In one example, the protein level may be measured by an immunoassay (e.g., ELISA assay or any antibody-based method known in the art).

In some aspects, the inhibitory polynucleotide (e.g., ASO or siRNA) comprises one or more modified nucleotides to improve certain properties of the nucleic acids, such as binding affinity, stability, and/or nuclease resistance. Accordingly, in some embodiments, nucleic acid comprises at least one nucleotide that is modified. In some aspects, the modified nucleotide comprises a sugar modification, a nucleic acid base modification, and/or a phosphate backbone modification. Modifications that are useful for optimizing inhibitory nucleic acids are described, e.g., in Freier & Altmann (1997), Nucl. Acid Res., 25, 4429-4443; Uhlmann (2000), Curr. Opinion in Drug Development, 3(2), 293-213; and Deleavey and Damha (2012), Chemistry and Biology, 19: 937-954, and U.S. Pat. Nos. 5,684,143, 5,858,988 and 6,291,438; Filippova et al. (2019), “Guide RNA modification as a way to improve CRISPR/Cas9-based genome-editing systems”, Biochimie., 167:49-60.

Suitable antisense RNA molecules, siRNA, miRNA, shRNA can be produced by standard methods of oligonucleotide synthesis or by ordering such molecules from a contract research organization or supplier by providing the polynucleotide sequence being targeted.

The specificity of the target sequence should generally be chosen with awareness that target mRNAs encoding a BMPR-1A or BMPR-2 protein can share similar sequences with non-target mRNAs encoding other gene products. Care should be taken to select a target sequence that has low sequence homology to other genes in the genome to allow for gene-specific knockdown. Where a gene has multiple forms, to achieve sufficient knockdown of gene expression, shRNA should target sequences shared among all isoforms of the target mRNA.

The potency and specificity of a candidate antisense molecule can be determined using cells expressing the BMPR-1A or BMPR-2 gene product to be targeted, measuring the degree of knockdown of the target with the degree of knockdown of other proteins that are normally manufactured by the cells in culture. The expression of BMPR-1A or BMPR-2 protein from the gene or mRNA being targeted can be assessed and quantified, for example, by enzyme-linked immunosorbent assay or Western blot. Ideal candidates will have high potency for inhibiting BMPR-1A or BMPR-2 expression (measured as a decreased production of the protein, compared with untreated controls) and a low potency for inhibiting expression of other proteins that are functionally unrelated to BMPR-1A or BMPR-2 (measured as a substantially unaltered production of such proteins, compared with untreated controls).

Depending on whether transient or stable expression is desired one can select an appropriate delivery vector. Examples of delivery vectors that may be used with the present disclosure are viral vectors, plasmids, exosomes, liposomes, bacterial vectors, or nanoparticles. Exemplary viral vectors include adenovirus vector, adeno-associated viral vector (AAV), retrovirus vector, lentivirus vector.

### 3.2.1 siRNA/shRNA

siRNA and shRNA are involved in the RNA interference (RNAi) pathway where they can induce degradation of a target RNA. Methods for constructing siRNAs useful for inhibiting target RNAs are known to those of skill in the art, see e.g., Fire et al. (1998), “Potent and specific genetic interference by double-stranded RNA in Caenorhabditis elegans”, Nature, 391:806-811; Elbashir et al. (2001), “Duplexes of 21-nucleotide RNAs mediate RNA interference in cultured mammalian cells”, Nature, 411:494-498; Brummelkamp (2002), “A System for Stable Expression of Short Interfering RNAs in Mammalian Cells”, Science, 296:550-553; Wittrup and Lieberman (2015), “Knocking down disease: a progress report on siRNA therapeutics”, Nature Rev Genet., 16:543-552; Vickers et al. (2003), “Efficient Reduction of Target RNAs by Small Interfering RNA and RNase H-dependent Antisense Agents”, J. Biol. Chem., 278:7108-7118. siRNAs comprise a sense strand and a complementary antisense strand annealed together by standard Watson Crick base pairing interactions. The sense strand may comprise a nucleic acid sequence that is identical to a target sequence contained within a target RNA, and the antisense strand may comprise a nucleic acid sequence that is complementary to a target sequence contained within the target RNA. In cells, the sense strand is degraded by RISC and the antisense strand directs RISC to an mRNA that has a complementary sequence. A protein called Ago2 in the RISC then cleaves the mRNA, or in some cases, represses translation of the mRNA, thus, leading to its destruction and an eventual reduction in the protein encoded by the mRNA. Thus, the siRNA leads to targeted gene silencing.

A short hairpin RNA or small hairpin RNA (shRNA) is an artificial RNA molecule that is converted to siRNA and, thus, can be used to silence target gene expression via the same (RNAi) pathway described above. See, e.g., Fire et. al., Nature 391:806-811, 1998; Elbashir et. Al., Nature 411:494-498, 2001; Chakraborty et al. Mol Ther Nucleic Acids 8:132-143, 2017; Bouard et al., Br. J. Pharmacol. 157:153-165, 2009. In the case of the shRNA, the sense and antisense strand are covalently linked by a single-stranded loop region, and the shRNA is converted into a siRNA by a cleavage event mediated by the enzyme Dicer. The loop region may be between 2 and 12 nucleotides in length. In some cases, the loop region is from 4 to 10 nucleotides in length. Details on the structure of shRNAs can be found, for example, in Paddison et al. (2002), “Short hairpin RNAs (shRNAs) induce sequence-specific silencing in mammalian cells”, Genes Dev., 16(8):948-958; Brummelkamp (2002), Science, 296:550-553; and Yu et al. (2002), “RNA interference by expression of short-interfering RNAs and hairpin RNAs in mammalian cells”, Proc Natl Acad Sci USA, 99:6047-6052).

In some embodiments, the siRNA or shRNA is 15-100, e.g., 15-50, e.g., 16-30, e.g., 19-25 nucleotides in length. In some embodiments, the siRNA or shRNA is 21 nucleotides in length. In some embodiments, the siRNA or shRNA comprises at least 15 contiguous nucleotides identical to SEQ ID NO: 1 or SEQ ID NO: 2.

In some aspects, the siRNA or shRNA comprises a sense strand and an antisense strand, where the antisense strand includes a region that is identical or substantially identical to a target sequence of the human BMPR-1A or BMPR-2 polynucleotide and the sense strand includes a region that is complementary or substantially complementary to a region of the antisense strand.

In some embodiments, the siRNA or shRNA comprises an overhang on the sense strand and/or the antisense strand. The overhang may be at the 5′ end and/or the 3′ end of either of the strands. The overhang can have any nucleotide sequence and may be 1-10 nucleotides in length, e.g., 2-6, e.g., 2-4 nucleotides in length.

### 3.2.2 Antisense Oligonucleotides

RNase H-dependent antisense oligonucleotides (ASOs) are single-stranded, chemically modified oligonucleotides that bind to complementary sequences in target mRNAs and reduce gene expression both by RNase H-mediated cleavage of the target RNA and by inhibition of translation by steric blockade of ribosomes.

RNase H is an endonuclease enzyme that catalyzes the cleavage of RNA in an RNA:DNA duplex. The most well studied endogenous function for this enzyme is the removal of Okazaki fragments (small RNAs) used to prime the DNA duplication during cell division. In some embodiments, to target the mRNA transcript of the BMPR-1A or BMPR-2 gene for degradation, a nucleic acid (e.g., DNA oligonucleotide) capable of hybridizing to a portion of the mRNA may be administered to the subject. Once inside the cell, the DNA oligonucleotide base pairs with its targeted mRNA transcript. RNase H may bind to the resulting duplex and cleave the mRNA transcript at one or more places. The DNA oligonucleotide may further bind to other mRNA transcripts to target them for RNase H degradation. Thus, the expression of the BMPR-1A or BMPR-2 gene may be greatly reduced in a subject with dementia.

The DNA oligonucleotide capable of hybridizing to an mRNA transcript of a BMPR-1A or BMPR-2 gene may contain, e.g., between 12 and 30 nucleotides (e.g., 12, 14, 16, 18, 20, 22, 24, 26, 28, or 30 nucleotides). In some embodiments, the DNA oligonucleotide may be 100% identical to a portion of a BMPR-1A or BMPR-2 polynucleotide. In other embodiments, the ASO may be less than 100% identical (e.g., 95%, 90%, 85%, 80%, 75%, or 70% complementarity) to a portion of a BMPR-1A or BMPR-2 polynucleotide, but can still form a stable RNA:DNA duplex for the RNase H to cleave the mRNA transcript. The ASO may bind to the 5′ UTR or the 3′ UTR of the mRNA transcript of the BMPR-1A or BMPR-2 gene.

### 3.2.3 miRNA

A microRNA (miRNA) is a small non-coding RNA molecule that functions in RNA silencing and post-transcriptional regulation of gene expression. miRNAs base pair with complementary sequences within the mRNA transcript. As a result, the mRNA transcript may be silenced by one or more of the mechanisms such as cleavage of the mRNA strand, destabilization of the mRNA through shortening of its poly(A) tail, and decrease translation efficiency of the mRNA transcript into proteins by ribosomes. In some embodiments, miRNAs resemble the siRNAs of the shRNA pathway, except that miRNAs derive from regions of RNA transcripts that fold back on themselves to form short hairpins, which are also called pri-miRNA. Once transcribed as pri-miRNA, the hairpins are cleaved out of the primary transcript in the nucleus by an enzyme called Drosha. The hairpins, or pre-miRNA, are then exported from the nucleus into the cytosol. In the cytosol, the loop of the hairpin is cleaved off by an enzyme called Dicer. The resulting product is now a double strand RNA with overhangs at the 3′ end, which is then incorporated into RISC. Once in the RISC, the second strand is discarded and the miRNA that is now in the RISC is a mature miRNA, which binds to mRNAs that have complementary sequences.

The difference between miRNAs and siRNAs from the shRNA pathway is that base pairing with miRNAs comes from the 5′ end of the miRNA, which is also referred to as the seed sequence. Since the seed sequence is short, each miRNA may target many more mRNA transcript. In some embodiments, an miRNA targeting BMPR-1A or BMPR-2 may be used in methods described herein. Suitable miRNAs are described in e.g., Lowery et al. (2016), “A survey of strategies to modulate the bone morphogenetic protein signaling pathway: current and future perspectives,” Stem Cells Int.; 2016:7290686. Exemplary miRNA can also be found e.g., in the “miRTarBase” database available at mirtarbase.cuhk.edu.cn/php/idex.php, which includes experimentally validated microRNA-target interactions.

### 3.2.4 CRISPR/CAS Systems

In some embodiments, the knocking out or knocking down of the BMPR-1A and/or BMPR-2 gene is performed using a gene editing system such as the CRISPR/Cas system. See Sanders and Joung, Nature Biotechnol 32:347-355, 2014, Huang et al., J Cell Physiol 10:1-17, 2017 and Mitsunobu et al., Trends Biotechnol 17:30132-30134, 2017. The “CRISPR/Cas” system refers to a widespread class of bacterial systems for defense against foreign nucleic acid. CRISPR/Cas systems include type I, II, and III sub-types. Any CRISPR/Cas system that is capable of altering a target polynucleotide sequence in a cell can be used in methods described here. Wild-type type II CRISPR/Cas systems use the RNA-mediated nuclease, for example, Cas9, in complex with guide and activating RNA to recognize and cleave foreign nucleic acid. In nature, many CRISPR systems include transactivating crisp RNA (tracrRNA), which binds the Cas endonuclease, and crisp RNA (crRNA), which binds to the DNA target sequence. Some CRISPR systems (e.g., CRISPR Cas12a/Cpf1) require only crRNA. In research and biomedical applications it is more typical to use a chimeric single guide RNA (“sgRNA”), which is a crRNA-tracrRNA fusion that binds both the Cas endonuclease and the DNA target sequence (e.g., BMPR-1A or BMPR-2 sequence). It will be understood that, except where apparent from context, reference to a “gRNA” includes any suitable guide RNA with appropriate binding specificity (e.g., a sgRNA, crRNA, or other RNA that binds to a gene encoding BMPR-1A or BMPR-2). The most commonly used sgRNA's comprise a nucleic acid sequence approximately 20 nucleotides in length. Methods for designing sgRNAs that target a specified target sequence are well known in the art. See e.g., Doench et al. (2016), Optimized sgRNA design to maximize activity and minimize off-target effects of CRISPR-Cas9″, Nat. Biotechnol. 34:184-191; Horlbeck et al. (2016), “Compact and highly active next-generation libraries for CRISPR-mediated gene repression and activation, eLife. 5, e19760 (2016); Cui et al., “Review of CRISPR/Cas9 sgRNA Design Tools. Interdiscip. Sci. 2018, 10:455-465; and Kiani et al. (2015), “Cas9 gRNA engineering for genome editing, activation and repression”, Nat Methods 2015; 12:1051-4.

Aspects of the invention relate to a nucleic acid that is a guide RNA (gRNA) that targets a polynucleotide encoding BMPR-1A or BMPR-2. In some aspects, introduction of the gRNA in a cell expressing BMPR-1A inhibits expression of BMPR-1A. In some aspects, introduction of the gRNA in a cell expressing BMPR-2 inhibits expression of BMPR-2. In some embodiments, the gRNA is of 20 nucleotides in length. In some embodiments, the gRNA comprises at least 12, at least 15, or at least 20 nucleotides identical to SEQ ID NO: 1 or SEQ ID NO: 2. In some cases, the guide RNA is an sgRNA. In some embodiments, the gRNA comprises at least 8, at least 9, at least 10, at least 11, at least 12, at least 13, at least 14, at least 15, at least 16, at least 17, at least 18, at least 19, or at least 20 contiguous nucleotides identical to SEQ ID NO: 1 or SEQ ID NO: 2.

In some aspects, the invention relates to a CRISPR/Cas system, where the system comprises a Cas protein and a guide RNA (e.g., an sgRNA) as described above. The sgRNA and Cas can be expressed from the same or different vectors of the system. Cas proteins and their amino acid sequence are well known in the art. The Cas protein used in the methods described herein can be a naturally occurring Cas protein or a functional derivative thereof. A “functional derivative” includes, but are not limited to, fragments of a native sequence and derivatives of a native sequence polypeptide and its fragments, provided that they have a biological activity in common with the corresponding native sequence polypeptide. A biological activity contemplated herein is the ability of the functional derivative to hydrolyze a DNA substrate (e.g., a BMPR-1A or BMPR-2 gene) into fragments. The term “derivative” encompasses both amino acid sequence variants of polypeptide, covalent modifications, and fusions thereof. Suitable derivatives of a Cas protein or a fragment thereof include but are not limited to mutants, fusions, or covalent modifications of Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. An exemplary Cas9 protein is the Streptococcus pyogenes Cas9 protein. The amino acid sequence of S. pyogenes Cas9 protein may be found in the SwissProt database under accession number Q99ZW2. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep 24; 110(39):15644-9; Sampson et al., Nature. 2013 May 9; 497(7448):254-7; and Jinek, et al., Science. 2012 Aug. 17; 337(6096):816-21.

In some cases, the gRNA binds to a target sequence that is contiguous with a protospacer adjacent motif (PAM) recognized by the Cas protein. For example, Cas9 generally requires the PAM motif NGG for activity. Thus, in some systems, certain target sequences will be preferred based on the proximity of the target sequence to a PAM. However, some Cas proteins, including variants of Cas9, have flexible PAM requirements (see e.g., Legut et al., 2020, “High-Throughput Screens of PAM-Flexible Cas9”, Cell Reports 30:2859-2868; Gleditzsch et al., 2019, PAM identification by CRISPR-Cas effector complexes: diversified mechanisms and structures. RNA Biol. 2019 April; 16(4): 504-517) and other Cas proteins are PAM-independent (e.g., Cas14a1). Exemplary PAMs are described, e.g., in Zhao et al. (2017), CRISPR-offinder: a CRISPR guide RNA design and off-target searching tool for user-defined protospacer adjacent motif. Int J Biol Sci; 13(12):1470-1478.

### 3.3 Selective inhibitors of BMPR-1A and/or BMPR-2

As described in the Examples below (see Example 5) single-cell analysis of key genes involved in the BMP pathway indicated that BMPR-1A and BMPR-2 are upregulated in Tg-SwDI mice compared to WT mice. Thus, in some embodiments, it is desirable to select agents that achieve selective inhibition of signaling mediated by BMPR-1A and/or BMPR-2. In some embodiments, agents suitable for use in the methods described herein inhibit signaling of BMPR-1A and/or BMPR-2 to a greater extent than the signaling of other BMPRs, such as for example, ActR-1A, BMPR-1B, ActR-2A, and ActR-2B.

Agents that inhibit signaling of BMPR-1A and/or BMPR-2 but do not inhibit signaling of other BMPRs (such as ActR-1A), or agents that signaling of BMPR-1A and/or BMPR-2 to a greater extent than signaling of other BMPRs (such as ActR-1A) can be designed based on differences in sequence and structure of BMPR-1A and/or BMPR-2 and other BMPRs (e.g., ActR-1A) proteins and their corresponding genes and mRNAs. For example, an alignment of BMPR-1A and ActR-1A mRNA sequences can identify non-identical or low identity nucleotide sequences that can be used to design shRNAs or other nucleic acid agents that associate with BMPR-1A mRNA but not ActR-1A sequences. Likewise, aligning BMPR-1A and ActR-1A amino acid sequences can identify divergent regions and antibodies or other binding agents can be produced to specifically bind the BMPR-1A protein. Likewise, small molecule agents can be identified (by rational drug design or screening) that specifically inhibit BMPR-1A and/or BMPR-2 activity, or inhibit BMPR-1A and/or BMPR-2 activity to a greater degree that ActR-1A activity.

The term “an agent that inhibits BMPR-1A and/or BMPR-2 activity but does not significantly inhibit activity of other BMPRs” as used herein, refers to an agent that is capable of specifically binding and inhibiting the activity of BMPR-1A and/or BMPR-2 such that minimal BMPR-1A and/or BMPR-2 activity is detected in vivo or in vitro; while the agent causes no significant decrease in activity of other BMPRs (such as ACTR-1A) under the same conditions. For example, an agent that inhibits activity of BMPR-1A and/or BMPR-2 can specifically bind to BMPR-1A and/or BMPR-2 and fully or significantly inhibit BMPR-1A and/or BMPR-2 activity in vivo or in vitro. In some cases, a BMPR-1A and/or BMPR-2 inhibitor can be identified by its ability to preferentially bind to the BMPR-1A and/or BMPR-2 gene or a BMPR-1A and/or BMPR-2 gene product, and fully inhibit signaling mediated by BMPR-1A and/or BMPR-2. In some cases, inhibiting signaling includes inhibiting the expression or activity of BMPR-1A and/or BMPR-2. Inhibition of BMPR-1A occurs when BMPR-1A activity, when exposed to an agent, is at least about 70% less, for example, at least about 75%, 80%, 90%, or 95% less than BMPR-1A activity in the presence of a control or in the absence of the agent. Inhibition of BPMR-2 occurs when BPMR-2 activity, when exposed to an agent, is at least about 70% less, for example, at least about 75%, 80%, 90%, or 95% less than BPMR-2 activity in the presence of a control or in the absence of the agent. No significant decrease in activity of other BMPRs (e.g., ACTR-1A) occurs, i.e., activity of other BMPRs (e.g., ACTR-1A), upon exposure to the agent, is at least about 90%, for example, at least 95%, 96%, 97%, 98%, 99%, or 100%, in comparison to activity of the BMPRs in the absence of the agent. As set forth herein, the agent can include small molecules (i.e., a molecule having a formula weight of 1000 Daltons or less), such as small molecule chemical inhibitors or large molecules, such as siRNA, shRNA, antisense oligonucleotides, or proteins.

Determining the effect of the agent on BMPR-1A and/or BPMR-2 and other BMPRs activity can be measured using one or more methods known in the art, including but not limited to, half maximal inhibitory concentration (IC₅₀), dissociation constant (K_{D}), and inhibitor constant (K_{I}). For example, IC₅₀ is a measure of the effectiveness of a substance in inhibiting a specific biological or biochemical function. This value indicates the concentration of the substance needed to inhibit a given biological process (or component of the biological process) by half. The IC₅₀ values are typically expressed as molar concentration. According to the Food and Drug Administration (FDA), IC₅₀ represents the concentration of a drug required for 50% inhibition in vitro. In one embodiment, an agent that inhibits BMPR-1A and/or BPMR-2 activity but does not significantly inhibit activity of other BMPRs (such as ACTR-1A) has an IC₅₀ that is at least about 2-fold, 5-fold, 10- fold, 50-fold, 75-fold, or 100-fold, lower than the concentration of the agent required to effect activity of another BMPR under the same conditions. In another embodiment, the IC₅₀for the agent to inhibit BMPR-1A and/or BPMR-2 activity is at least about 25%, 50%, 75%, 80%, 85%, 90%, 95%, 96%, 97%, 98%, or 99%, lower than the IC₅₀ for the agent to inhibit the activity of another BMPRs (e.g., ACTR-1A).

In some embodiments, the effect of the agent on BMPR-1A and/or BPMR-2 and other BMPRs activity can be determined by calculating the equilibrium dissociation constant (K_{D}) of the agent to each BMPR. For example, an agent that inhibits the activity of BMPR-1A and/or BPMR-2 but does not significantly inhibit activity of other BMPRs has a K_{D} that is at least about 2-fold, 5-fold, 10-fold, 50-fold, or 100-fold lower than the K_{D} of the agent to another BMPR (e.g., ACTR-1A) under the same conditions. In one embodiment, the K_{D}for the agent to BMPR-1A and/or BPMR-2 is at least about 25%, 50%, 75%, 80%, 85%, 90%, 95%, 96%, 97%, 98%, or 99%, lower than the K_{D} for the agent to another BMPR (such as ACTR-1A). In a preferred embodiment, the K_{D} is lower for the agent to BMPR-1A and/or BPMR-2 as compared to the K_{D} of the agent another BMPR. Said differently, the equilibrium dissociation constant of the agent to other BMPRs is greater than the equilibrium dissociation constant of the agent to BMPR-1A and/or BPMR-2. In one embodiment, the agent can include an antibody having a K_{D} value in the micromolar (10⁻⁶) to nanomolar (10⁻⁷ to 10⁻⁹) range. In another embodiment, the agent can include an antibody having a K_{D} in the nanomolar range (10⁻⁹) to the picomolar (10⁻¹²) range. In yet another embodiment, the agent can have a nanomolar (nM) equilibrium dissociation constant to BMPR-1A and a micromolar (μM) equilibrium dissociation constant to ACTR-1A. US Patent Publication No. US20120071477 describes kinase inhibition assays in which a compound at a single concentration (2,000 nM) can be used to inhibit ATP pocket binding.

In some embodiments, the effect of the agent on BMPR-1A and/or BPMR-2 and other BMPR activity can be determined by calculating the inhibitor constant (K_{I}) of the agent to each BMPR. The K_{I} is an indication of how potent an inhibitor is; it is the concentration required to produce half maximum inhibition. The lower the K_{I}, the greater the binding affinity between the agent and the BMPR gene. For example, an agent that inhibits the activity of BMPR-1A and/or BPMR-2 but does not significantly inhibit activity of another BMPR (e.g., ACTR-1A) has a K_{I} that is at least about 2-fold, 5-fold, 10- fold, 50-fold, 75-fold, or 100-fold lower than the K_{I} of the agent (to ACTR-1A) under the same conditions. In one embodiment, the K_{I} for the agent to BMPR-1A and/or BPMR-2 is at least about 25%, 50%, 75%, 80%, 85%, 90%, 95%, 96%, 97%, 98%, or 99%, lower than the K_{I} for the agent to other BMPRs (such as ACTR-1A). In one embodiment, the K_{I} is lower for the agent to BMPR-1A as compared to the K_{I} of the agent to ACTR-1A. Said differently, the inhibitor constant of the agent to ACTR-1A is greater than the inhibitor constant of the agent to BMPR-1A. For example, an agent that inhibits activity of BMPR-1A can bind to BMPR-1A and significantly inhibit BMPR-1A activity in vivo or in vitro. In some cases, a BMPR-1A inhibitor can be identified by its ability to preferentially bind to BMPR-1A and fully inhibit activity of BMPR-1A. Inhibition of BMPR-1A occurs when BMPR-1A activity, when exposed to an agent of the invention, is at least about 70% less, for example, at least about 75%, 80%, 90%, 95%, 96%, 97%, 98%, 99% less, or totally inhibited, in comparison to BMPR-1A activity in the presence of a control or in the absence of the agent. In one embodiment, the K_{I} is lower for the agent to BPMR-2 as compared to the K_{I} of the agent to other BMPRs (e.g., ACTR-1A). Said differently, the inhibitor constant of the agent to other BMPRs is greater than the inhibitor constant of the agent to BPMR-2. For example, an agent that inhibits activity of BPMR-2 can bind to BPMR-2 and significantly inhibit BPMR-2 activity in vivo or in vitro. In some cases, a BPMR-2 inhibitor can be identified by its ability to preferentially bind to BPMR-2 and fully inhibit activity of BPMR-2. Inhibition of BPMR-2 occurs when BPMR-2 activity, when exposed to an agent of the invention, is at least about 70% less, for example, at least about 75%, 80%, 90%, 95%, 96%, 97%, 98%, 99% less, or totally inhibited, in comparison to BPMR-2 activity in the presence of a control or in the absence of the agent. No significant decrease in activity for other BMPRs occurs. Activity of other BMPRs upon exposure to the agent, is at least about 90%, for example, at least 95%, 96%, 97%, 98%, 99%, or 100%, in comparison to activity of other BMPRs in the absence of the agent.

The term “an agent that inhibits activity of BMPR-1A and/or BMPR-2 to a greater extent than the activity of other BMPRs” as used herein, refers to an agent that is capable of binding and inhibiting the activity of BMPR-1A and/or BMPR-2 significantly more than the agent's effect on inhibiting the activity of other BMPRs (such as ACTR-1A) under the same conditions. For example, an agent that inhibits activity of BMPR-1A and/or BMPR-2 to a greater extent than inhibiting the activity of other BMPRs, occurs when BMPR-1A and/or BMPR-2 activity, when exposed to an agent of the invention, is at least about 10% less, for example, at least about 15%, 20%, 30%, 40%, or 50% less, than the activity of other BMPRs under the same conditions in vitro or in vivo. In one embodiment, an agent inhibits the activity of BMPR-1A to a greater extent than the activity of ACTR-1A, when the activity of BMPR-1A observed is at least 10% less than the activity of ACTR-1A under the same conditions. In another embodiment, an agent inhibits the activity of BMPR-1A to a greater extent than ACTR-1A activity, when at least 2-fold, 5-fold, 10-fold, 20-fold, 50-fold, or 100-fold less BMPR-1A activity is observed as compared to ACTR-1A activity under the same conditions. In one embodiment, an agent inhibits the activity of BPMR-2 to a greater extent than the activity of another BMPR, when the activity of BPMR-2 observed is at least 10% less than the activity of another BMPR under the same conditions. In another embodiment, an agent inhibits the activity of BPMR-2 to a greater extent than activity of another BMPR, when at least 2-fold, 5-fold, 10-fold, 20-fold, 50-fold, or 100-fold less BPMR-2 activity is observed as compared to activity of other BMPRs under the same conditions. The extent of inhibition (e.g., comparing BMPR-1A activity to ACTR-1A activity) can be determined using one or more methods known in the art, including but not limited to “Percent Of Control (POC)” or “Normalized Percent Inhibition (NPI)”. POC and NPI are methods that normalize data and are often used when comparing multiple agents (e.g., various antibodies or small molecules) against multiple targets (e.g., BMPR-1A and ACTR-1A). For example, POC is a method that corrects for plate-to-plate variability (for example in high-throughput drug screening) by normalizing an agent's measurement relative to one or more controls present in the plate. Raw measurements for each agent can be divided by the “average” of within-plate controls. NPI is a control-based method in which the difference between the agent measurement and the mean of the positive controls is divided by the difference between the means of the measurements on the positive and the negative controls (Malo et al., Nature Biotechnology, Vol. 24, 167-175 (2006)). By normalizing the extent of inhibition observed, accurate conclusions can be made regarding which agent(s) are effective at inhibiting the activity of each target under investigation.

### 4. Methods of Increasing the Self-Renewal Rate of Cells

In another aspect, methods of increasing the self-renewal rate of cells (e.g., neural stem cells) are provided. In some embodiments, the method comprises contacting the cells (e.g., neural stem cells) with an agent that inhibits signaling of BMPR-1A and/or BMPR-2. In some embodiments, contacting includes culturing the cells (e.g., neural stem cells) in the presence of an agent that inhibits expression or activity of BMPR-1A and/or BMPR-2. In yet another aspect, compositions are provided that comprise cell populations in which the cells have been incubated with agent that inhibits signaling of BMPR-1A and/or BMPR-2 (e.g., according to the methods disclosed herein).

In some embodiments, the cell is a multipotent cell. As used herein, “multipotent” refers to the ability of a cell (e.g., an adult stem cell) to form multiple cell types of one lineage, i.e., to differentiate into more specialized cell types. For example, neural stem cells are able to form, for example, neurons and glial cells (e.g., astrocytes and oligodendrocytes). In some embodiments, the cell is a neural stem cell. In some embodiments, the cell is a neural progenitor cell. The neural progenitor cell may be derived from a neural stem cells. The neural progenitor cell may be able to form neurons, astrocytes, and oligodendrocytes. In some embodiments, the neural progenitor cell may be able to form different types of neurons.

Cells (e.g., neural stem cells or neural progenitor cells) can be derived from, for example, from a brain tissue obtained from a subject. The cells may be, for example, derived from tissue obtained from the subventricular zone (SVZ), or the dentate gyrus of the hippocampus). In some cases, the cell may be obtained by differentiation of the cells derived from human embryonic stem cells (hESCs), induced pluripotent stem cells (iPSCs), human adult stem cells, human hematopoietic stem cells, human mesenchymal or stem cells (hMSCs). Hematopoietic stem cells or hematopoietic progenitor cells, may be harvested by apheresis, leukocytapheresis, density gradient separation, bone marrow biopsy, fetal liver biopsy, or cord blood.

In some embodiments, the cells are derived from humans or from non-human mammals. Exemplary non-human mammals include, but are not limited to, mice, rats, cats, dogs, rabbits, guinea pigs, hamsters, sheep, pigs, horses, bovines, and non-human primates. In some embodiments, a cell is from an adult human or non-human mammal.

In some embodiments, the cells are cultured in the presence of an agent that inhibits expression or activity of BMPR-1A and/or BMPR-2. In some embodiments, the agent binds to BMPR-1A and/or BMPR-2. In some embodiments, the agent is a small molecule inhibitor, e.g., LDN19389. In some embodiments, the agent is an antibody. In some embodiments, the agent is a nucleic acid, e.g., an siRN or ASO targeting a BMPR-1A and/or BMPR-2 mRNA. In some embodiments, the cells are contacted with a composition comprising an agent that inhibits expression or activity of BMPR-1A and/or BMPR-2 as disclosed herein. In some embodiments, the composition comprising the inhibitory agent comprises an acceptable carrier and/or excipients.

In some embodiments, the methods of increasing the self-renewal rate of cells comprise culturing the cells in the presence of the inhibitory agent under suitable conditions for the maintenance of the cells. It will be recognized by a person of ordinary skill in the art that the culture conditions will vary depending upon the cell type and the origin of the cell being cultured. Exemplary cell culture conditions are described in the art. See, e.g., Picot, Human Cell Culture Protocols (Methods in Molecular Medicine), 2010 ed., Davis, Basic Cell Culture, 2002 ed., and Lee et al., Int J Stem Cells, 2011, 4:9-17. For example, in some embodiments, the cells are cultured in the presence of one or more cell culture supplements, e.g., growth factors (e.g., bFGF or LIF), small molecule inhibitors, amino acids, or antibiotics. Examples of mediums and reagents that find particular use in the culturing of neural stem cells, neural progenitor cells and neurons may be found in the Example section below.

In some embodiments, culturing the cells with the inhibitory agent increases the self-renewal rate of cells by at least 10%, at least 20%, at least, 30%, at least 40%, at least, 50%, at least, 60%, at least 70%, at least 80%, at least 90%, at least 95% or more. In some embodiments, the cells are cultured in the presence of the inhibitory agent for at least 1, 2, 3, 4, 5, 6, 7, 8, 9, 10, 15, 20 or more cell divisions. In some embodiments, culturing the cells with inhibitory agent promotes the proliferation of the cells for at least 1, 2, 3, 4, 5, 6, 7, 8, 9, 10, 15, 20 or more cell divisions. In some embodiments, self-renewal rate of cells (e.g., neural stem cells) is measured as described in the Examples section below (see e.g., Example 6). Methods of detecting and quantifying neural stem cell self-renewal are also described in the art. See, e.g., Molofsky, 2005; Hu & Smyth, 2009; Pastrana, Silva-Vargas, & Doetsch, 2011.

In some embodiments, the methods of increasing the self-renewal rate of cells (e.g., neural stem cells) are performed in vitro or ex vivo. In some embodiments, the methods of increasing the self-renewal rate of cells (e.g., neural stem cells) are performed on cells that are obtained from a donor (e.g., a human donor). In some embodiments, the cells are optionally further expanded before being administered to a patient. In some embodiments, the cells are autologous to the patient (i.e., the cells are administered to the same patient from whom the cells were obtained). In some embodiments, the cells are allogeneic to the patient. Thus, in some embodiments, the methods and resulting cell compositions find application in regenerative medicine. In some embodiments, the methods of promoting or maintaining the proliferation of cells and resulting cell compositions can be used to grow new organoids and tissues from the cells. In some embodiments, the methods and cell compositions can be used for the transplantation of cells or tissues into a patient.

In some embodiments, a composition comprising a population of cells as disclosed herein, e.g., a composition comprising a population of cells (e.g., neural stem cells) produced according to a method disclosed herein, is used for a regenerative medicine application, such as cell therapy. In some embodiments, a composition comprising a population of cells produced according to a method disclosed herein can be used in a method of cell therapy for the treatment of a neurodegenerative disease, such as AD.

### 5. Pharmaceutical Compositions

For administering an agent as disclosed herein (e.g., an agent that inhibits signaling mediated by BMPR-1A and/or BMPR-2), in some embodiments, the agent is formulated as a pharmaceutical composition. In some embodiments, the pharmaceutical composition comprises an inhibitor of BMPR-1A and a pharmaceutically acceptable excipient or carrier. In some embodiments, the pharmaceutical composition comprises an inhibitor of BMPR-2 and a pharmaceutically acceptable excipient or carrier. Guidance for preparing formulations for use according to the present disclosure is found in, for example, in Remington: The Science and Practice of Pharmacy, 21st Edition, Philadelphia, Pa., Lippincott Williams & Wilkins, 2005; and Gibson, Pharmaceutical Preformulation and Formulation: A Practical Guide from Candidate Drug Selection to Commercial Dosage Form, 2001, Interpharm Press. The pharmaceutical compositions described herein are designed for delivery to subjects in need thereof by any suitable route or a combination of different routes. The following methods and excipients are merely exemplary and are in no way limiting.

In some embodiments, a pharmaceutical composition comprises an acceptable carrier and/or excipients. A pharmaceutically acceptable carrier includes any solvents, dispersion media, or coatings that are physiologically compatible and that preferably does not interfere with or otherwise inhibit the activity of the therapeutic agent. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known and generally described in, for example, Remington: The Science and Practice of Pharmacy, supra. Various pharmaceutically acceptable excipients are well-known in the art and can be found in, for example, Handbook of Pharmaceutical Excipients (5^{th} ed., Ed. Rowe et al., Pharmaceutical Press, Washington, D.C.).

In some embodiments, the pharmaceutical composition is formulated for administration by injection. Pharmaceutical preparations for administration by injection can be formulated by dissolving, suspending or emulsifying the compound in an aqueous or nonaqueous solvent. In some approaches, sterile injectable solutions can be prepared with the agent in the required amount and an excipient suitable for injection into a human patient. In some embodiments, the pharmaceutically and/or physiologically acceptable excipient is particularly suitable for administration to the brain. For example, a suitable carrier may be buffered saline or other buffers, e.g., HEPES, to maintain pH at appropriate physiological levels, stabilizing agents, adjuvants, diluents, or surfactants. For injection, the excipient will typically be a liquid. Exemplary pharmaceutically acceptable excipients include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution.

In some embodiments, the pharmaceutical composition is formulated for oral administration. Compositions for oral administration can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral administration can be obtained by mixing the compounds with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl-pyrrolidone (PVP).

In some cases, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel.

### 6. Administration Methodology and Dosage

### 6.1 Administration

Aspects of the invention include methods of administering a therapeutically effective amount of an agent that inhibits signaling by BMPR-1A and/or BMPR-2 for treating a neurodegenerative disease (e.g., AD) in a subject in need of treatment. Administration is not limited to a particular site or method. Any suitable route of administration or combination of different routes can be used, including, but not limited to, parenteral administration (e.g., intravenous, intramuscular, subcutaneous, or intradermal injection, local injection into the central nervous system (CNS)) or oral administration (e.g., in the form of a tablet or capsule). In some approaches, the agent is administered by injection, such as intravenous injection. In some approaches, the agent is administered to the brain.

### 6.2 Dosage and Effective Amounts

Dosage values may depend on the nature of the product and the severity of the condition. It is to be understood that for any particular subject, specific dosage regimens can be adjusted over time and in course of the treatment according to the individual need and the professional judgment of the person administering or supervising the administration of the compositions. Accordingly, dosage ranges set forth herein are exemplary only and are not intended to limit the scope or practice of the claimed composition.

The amount of agent administered will be an “effective amount” or a “therapeutically effective amount,” i.e., an amount that is effective, at dosages and for periods of time necessary, to achieve a desired result. A desired result would include an increase in the self-renewal rate of cells (e.g., neural stem cell, a neural progenitor cell), improvement of cognitive abilities, or a detectable improvement in a symptom associated with the neurodegenerative disease (e.g., AD) that improves patient quality of life. Alternatively, if the pharmaceutical composition is used prophylactically, a desired result would include a demonstrable prevention of one or more symptoms of the neurodegenerative disease (e.g., AD). A desired result would also include a delay in the onset or progression of preclinical AD to clinical AD. A therapeutically effective amount of such a composition may vary according to factors such as the disease state (e.g., AD stage), age, sex, and weight of the individual, or the ability of the agent to elicit a desired response in the individual. Dosage regimens may be adjusted to provide the optimum response. A therapeutically effective amount is also one in which any toxic or detrimental effects of the viral vector are outweighed by the therapeutically beneficial effects.

For example, an effective dose is the dose that when administered for a suitable period of time, will slow e.g., by about 10% or more, by about 20% or more, e.g. by 30% or more, by 40% or more, or by 50% or more, in some instances by 60% or more, by 70% or more, by 80% or more, or by 90% or more, or halt cognitive decline, in a patient suffering from a neurodegenerative disease such as AD. In some embodiments, an effective amount or dose may not only slow or halt the progression of the disease condition but may also induce the reversal of the condition. For example, an effective dose is the dose that when administered for a suitable period of time, will improve the cognition in an individual with, for example, a neurodegenerative disease such as AD. An improvement in cognition may be observed as, for example, an improvement in memory. Improvements in memory may be readily assessed using any suitable method known in the art, e.g., by assaying retrieval-related brain activity (Buchmann A, et al. (2008) Prion protein M129V polymorphism affects retrieval-related brain activity. Neuropsychologia. 46(9):2389-402) or, e.g., by imaging brain tissue by functional magnetic resonance imaging (fMRI) following repetition priming with familiar and unfamiliar objects (Soldan A, et al. (2008), Global familiarity of visual stimuli affects repetition-related neural plasticity but not repetition priming. Neuroimage. 39(1):515-26; Soldan A, et al. (2008) Aging does not affect brain patterns of repetition effects associated with perceptual priming of novel objects. J Cogn Neurosci. 20(10):1762-76). Other examples include tests such as cognition test for measuring cognitive ability, e.g. attention and concentration, the ability to learn complex tasks and concepts, memory, information processing, visuospatial function, the ability to produce and understanding language, the ability to solve problems and make decisions, and the ability to perform executive functions; for example, the General Practitioner Assessment of Cognition (GPCOG) test (Brodaty et al. (2002), “The GPCOG: a new screening test for dementia designed for general practice,” Journal of the American Geriatrics Society, 50(3): 530-4), the Memory Impairment Screen, the Mini Mental State Examination (MMSE; Folstein et al. (1975) J. Psychiatric Research 12 (3): 189-198), the Delis-Kaplan Executive Functioning System test (Delis, Kaplan & Kramer (2001), “The Delis-Kaplan Executive Function System (D-KEFS) Examiners' Manual, San Antonio, Tex., The Psychological Corporation), and the like.

Delay in the onset or progression of AD may be further assessed by measuring in vivo molecular biomarkers relative to an initial baseline value. For example, a reduction in brain amyloid deposition may be measured, by assessing a change from baseline in composite cortical amyloid standard uptake value ratio (SUVR) using positron emission tomography (PET) imaging (Palmqvist S et al., 2015). In one embodiment, brain amyloid deposition relative to an initial baseline value is reduced. In another approach, tau may be measured. For example, by using PET and a suitable Tau tracer, total Tau and/or phosphorylated Tau may be measured. In one embodiment, the levels of CSF Tau or phosphorylated Tau are reduced relative to an initial baseline value. In some embodiments, effects on neuronal glucose metabolism, density and/or activity may be measured. The F-FDG PET signal in AD-affected brain regions has been shown to be associated with cognitive impairment, subsequent cognitive decline and neuropathology in AD and to progress over time in the clinical and preclinical stages of AD, and is a disease and treatment efficacy biomarker (Foster N Let al., 2007). In some embodiments, delay in the onset or progression of AD may be shown as slower decline in brain volume loss, as assessed by volumetric magnetic resonance imaging (vMRI) to measure a change from baseline in brain volume. vMRI can be used to measure a change in hippocampus, lateral ventricle, and total brain volume. In some embodiments, CSF amyloid-beta protein 40, amyloid-beta protein 42 levels may be measured. In one embodiment, CSF amyloid-beta protein 40 and/or amyloid-beta protein 42 ratio increases relative to an initial baseline value.

The effective amount of the compositions described herein can be determined by one of ordinary skill in the art. An effective amount of any of the compositions described herein will vary and can be determined by one of skill in the art through experimentation and/or clinical trials. In some embodiments, a daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. For composition comprising a viral vector for the delivery of polynucleotides quantification of genome copies (GC), vector genomes (VG), virus particles (VP), or infectious viral titer may be used as a measure of the dose contained in a formulation or suspension. Any method known in the art can be used to determine the GC, VG, VP or infectious viral titer of the virus compositions of the invention, including as measured by qPCR, digital droplet PCR (ddPCR), UV spectrophotometry, ELISA, next-generation sequencing, or fluorimetry as described in, e.g. in Dobkin et al., “Accurate Quantification and Characterization of Adeno-Associated Viral Vectors.” Front Microbiol 10: 1570-1583 (2019); Lock et al., “Absolute determination of single-stranded and self-complementary adeno-associated viral vector genome titers by droplet digital PCR.” Hum Gene Ther Methods 25: 115-125 (2014).

### 6.3 Combination Therapies

In some approaches, the methods and compositions of the present disclosure are used in combination with one or more additional agents and/or therapies, including any known, or as yet unknown, agents or therapies which help preventing development of, slowing progression of, reversing, or ameliorating the symptoms of a neurodegenerative disease (e.g., AD). The one or more additional agents and/or therapies may be administered and/or performed before, concurrent with, or after administration of the inhibitory agents described herein. The combined administration includes co-administration, using separate formulations or a single pharmaceutical formulation.

Examples of other agents that can be combined with the inhibitory agents described herein include, without limitation any one or more of acetylcholinesterase inhibitors such as donepezil, rivastigmine, and galantine, and the NMDA receptor antagonist memantine. Additional therapeutic approaches that may be combined with the methods described herein include administering BACE or y-Secretase inhibitors, and active or passive immune therapies targeting Aβ (see e.g., Wisniewski and Goni (2015), “Immunotherapeutic approaches for Alzheimer's disease,” Neuron 85, 1162-1176).

### 7. Patients and Treatable Conditions

Patients or subjects who are candidates for treatment with the methods and compositions described herein include those experiencing or having experienced one or more signs, symptoms, or other indicators of a disease or disorder described below. A “patient” or “subject” herein refers to any single animal, including, for example, a mammal, such as a human. In some embodiments, the patient to be treated is a human. In some embodiments, the patient has a neurodegenerative disease. The neurodegenerative disease may be Alzheimer's Disease (AD). The neurodegenerative disease may be Dementia. The neurodegenerative disease may be Parkinson's Disease. The neurodegenerative disease may be Amyotrophic Lateral Sclerosis (ALS); The neurodegenerative disease may be Down Syndrome. In some instances, the subject has AD. In some instances, the subject has Parkinson's Disease.

In some embodiments, the subjects have been diagnosed as having AD. In some approaches, patients are selected for treatment based on signs, symptoms, clinical phenotypes and/or biomarkers. Diagnostic criteria for AD, referred to as the NINCDS-ADRDA criteria (McKhann et al. (2011), “The diagnosis of dementia due to Alzheimer's disease: recommendations from the National Institute on Aging-Alzheimer's Association workgroups on diagnostic guidelines for Alzheimer's disease,” Alzheimers Dement, May; 7((3)):263-9), are known in the art and can be used for the diagnosis of AD. AD is generally characterized by symptoms which have a gradual onset over months to years, not sudden over hours or days. The NINCDS-ADRDA Alzheimer's Criteria specify different cognitive domains that may be impaired in AD including memory, language, perceptual skills, attention, constructive abilities, orientation, problem solving and functional abilities. Other diagnostic classification systems include the International Working Group (IWG) new research criteria for diagnosis of AD (Dubois B et al. Lancet Neurol 2007; 6(8):734-736), IWG research criteria, (Dubois et al. Lancet Neurol 2010; 9(11):1118-27), NIA/AA Criteria (Jack C R et al. Alzheimer's Dement 2011; 7(3):257-62), and DSM-5 criteria (American Psychiatric Association, DSM-5, 2013).

A variety of approaches and tools can be used to diagnose AD and measure disease progression, including conducting problem-solving, memory and other cognitive tests, as well as physical and neurologic examinations. For example, psychometric measures and cognitive screening tools may be used, such as the Mini-Mental State Exam (MMSE; Folstein et al. (1975) J. Psychiatric Research 12 (3): 189-198), and the Alzheimer's Disease Assessment Scale (ADAS), which is a comprehensive scale for evaluating patients with Alzheimer's Disease status and function (see, e.g., Rosen, et al. (1984) Am. J. Psychiatr., 141: 1356-1364). Cognitive impairment and decline in the diagnosis of MCI due to AD and AD dementia may be measured using a cognition and functional performance measure to track changes in the clinical stages of the disease, for example, using the Clinical Dementia Rating (CDR) Scale (see e.g., Hughes et al. (1982), “A new scale for the staging of dementia,” Br. J. Psychiatry, 140:566-572). Other cognitive screening tools include but are not limited to AMTS (Abbreviated Mental Test Score), Clock Drawing Test, 6-CIT (6-Item Cognitive Impairment Test), GPCOG (General Practitioner Assessment of Cognition), TYM (Test Your Memory), MoCA (Montreal Cognitive Assessment), ACE-R (Addenbrooke's Cognitive Examination—Revised), and the MIS (Memory Impairment Screen). These and other cognitive assessment tools are described in e.g., Sheenan (2012), “Assessment scales in dementia,”, Ther. Adv. Neurol. Disord, 5(6): 349-358. Delay in the onset or progression of preclinical AD may also be assessed relative to an initial baseline value using a sensitive cognitive measure to track changes in the preclinical stages of the disease, for example, using the Alzheimer's Prevention Initiative (API) preclinical composite cognitive (APCC) test battery (Langbaum J B et al., 2014). In some approaches, a diagnosis of dementia and/or AD can be determined by considering the results of several clinical tests in combination (see e.g., Grundman, et al., Arch Neurol (2004) 61:59-66).

In some cases, diagnosis may include magnetic resonance imaging (MRI) to determine progressive loss of gray matter in the brain (see, e.g., Whitwell et al. (2008) Neurology 70(7): 512-520; Frisoni, et al. (2010), “The clinical use of structural MRI in Alzheimer disease,” Nature Reviews Neurology 6, 67-77; Scheltens et al. (2002), “Structural magnetic resonance imaging in the practical assessment of dementia: beyond exclusion,” The Lancet Neurology 1, 13-21; Fennema-Notestine et al. (2009), “Structural MRI biomarkers for preclinical and mild Alzheimer's disease,” Human Brain Mapping 30, 3238-3253). In some cases, positron emission tomography (PET) imaging of the brain may be performed to determine if the individual has high levels of beta-amyloid. In some cases, single photon emission computed tomography (SPECT) can be used to diagnose AD. In some cases, lumbar puncture may be performed to determine levels of beta-amyloid and certain types of tau in cerebrospinal fluid (CSF). These include measurement of Tau, phosphorylated Tau (pTau), soluble amyloid precursor protein (sAPP)-α, sAPP-β, amyloid-beta protein 40, amyloid-beta protein 42 levels and/or C terminally cleaved APP fragment (APPneo). These methods are known in the art and described in e.g., Waldemar et al. (2007), “Recommendations for the diagnosis and management of Alzheimer's disease and other disorders associated with dementia: EFNS guideline,” European Journal of Neurology. 14 (1): e1-26; Blennow et al. (2015, “Amyloid biomarkers in Alzheimer's disease,” Trends Pharmacol Sci.; 36(5):297-309; Mallik et al. (2017), “Clinical Amyloid Imaging. Semin Nucl Med., 47(1):31-43.

In one aspect, administration of the compositions described herein at a very early stage of disease progression may provide superior therapeutic benefit. For example, treatment may be performed prior to the appearance of signs or symptoms of the disease. Thus, provided herein are methods and compositions for preventing development of the neurodegenerative disease (e.g., AD). In some approaches, the patient has no symptoms of AD. In some approaches, patients are assessed by genotyping to determine their individual genetics (e.g., by assessing the presence of risk alleles associated with neurodegenerative diseases described above) and associated risk of disease. Accordingly, in some approaches, at the time of first administration of the composition, the patient does not exhibit any of the clinical phenotypes of the disease.

### 8. EXAMPLES

### 8.1 Example 1. Approach and Methods

### 8.1.1 Statistical Analyses

In all the graphs, bars show average as central values and ±S.D. as error bars, unless otherwise specified. P values were calculated using ANOVA in analyses with 3 or more groups. Two-tailed t-tests were used in analyses comparing 2 groups, unless otherwise specified. For limiting dilution analyses, ELDA software was used to test inequality between multiple groups. Expected frequencies are reported, as well as the 95% confidence intervals (lower and upper values are indicated). *P<0.05, **P<0.01, ***P<0.001.

### 8.1.2 Brain Multianalyte Analysis

The different brain regions were lysed using cell lysis buffer (Cell signaling #9803) with PMSF (Cell signaling #8553) and complete mini EDTA free protease inhibitor followed by mechanical homogenation by Tissue Ruptor (Qiagen). The samples were centrifuged at 13000 rpm for 15 mins and protein concentration calculated by BCA. Normalized samples were analyzed by the Stanford Human Immune Monitoring Center using a Luminex mouse 38-plex analyte platform that screens 38 secreted proteins using a multiplex fluorescent immunoassay. Brain homogenates were run in duplicate (three biological replicates were analyzed).

### 8.1.3 Flow Cytometry

For single-cell RNA-sequencing, the subventricular zone of 4 mice from each genotype was micro-dissected and tissue digested using Liberase DH (Roche) and DNAse I (250 U/ml) at 37° C. for 20 minutes followed by trituration. Digested tissue was washed in ice-cold HBSS without calcium and magnesium, filtered through a 40-μm filter, and then stained with the following antibodies for 30 minutes: PacBlue-CD31 (Biolegend), PacBlue-CD45 (Biolegend), PacBlue-Ter119 (Biolegend), and FITC-CD24 (Biolegend). Sytox Blue was used for cell death exclusion and samples were sorted into 384 well plates prepared with lysis buffer using the Sony Sorter. For bulk RNA-sequencing, tissue was processed as above (4 replicates per genotype), cells were stained using an Anti-GLAST (ACSA-1) Microbead Kit (Miltenyi Biotec), and GLAST+ cells were separated using a positive selection scheme with a MACS separator.

### 8.1.4 Lysis Plate Preparation

Lysis plates were created by dispensing 0.4 μl lysis buffer (0.5 U Recombinant RNase Inhibitor (Takara Bio, 2313B), 0.0625% Triton™ X-100 (Sigma, 93443-100 ML), 3.125 mM dNTP mix (Thermo Fisher, R0193), 3.125 μM Oligo-dT30VN (IDT, 5′AGCAGTGGTATCAACGCAGAGTACT30VN-3′) (SEQ ID NO: 3) and 1:600,000 ERCC RNA spike-in mix (Thermo Fisher, 4456740)) into 384-well hard-shell PCR plates (Biorad HSP3901) using a Tempest or Mantis liquid handler (Formulatrix).

### 8.1.5 cDNA Synthesis and Library Preparation

cDNA synthesis was performed using the Smart-seq2 protocol [1,2]. Illumina sequencing libraries were prepared according to the protocol in the Nextera XT Library Sample Preparation kit (Illumina, FC-131-1096). Each well was mixed with 0.8 μl Nextera tagmentation DNA buffer (Illumina) and 0.4 μl Tn5 enzyme (Illumina), then incubated at 55° C. for 10 min. The reaction was stopped by adding 0.4 μl “Neutralize Tagment Buffer” (Illumina) and spinning at room temperature in a centrifuge at 3220×g for 5 min. Indexing PCR reactions were performed by adding 0.4 μl of 5 μM i5 indexing primer, 0.4 μM of 5 μM i7 indexing primer, and 1.2 μl of Nextera NPM mix (Illumina). PCR amplification was carried out on a ProFlex 2×384 thermal cycler using the following program: 1. 72° C. for 3 minutes, 2. 95° C. for 30 seconds, 3. 12 cycles of 95° C. for 10 seconds, 55° C. for 30 seconds, and 72° C. for 1 minute, and 4. 72° C. for 5 minutes.

### 8.1.6 Library Pooling, Quality Control, and Sequencing

Following library preparation, wells of each library plate were pooled using a Mosquito liquid handler (TTP Labtech). Pooling was followed by two purifications using 0.7× AMPure beads (Fisher, A63881). Library quality was assessed using capillary electrophoresis on a Fragment Analyzer (AATI), and libraries were quantified by qPCR (Kapa Biosystems, KK4923) on a CFX96 Touch Real-Time PCR Detection System (Biorad). Plate pools were normalized to 2 nM and equal volumes from 10 or 20 plates were mixed together to make the sequencing sample pool. PhiX control library was spiked in at 0.2% before sequencing. Single-cell libraries were sequenced on the NovaSeq 6000 Sequencing System (Illumina) using 2×100 bp paired-end reads and 2×8 bp or 2×12 bp index reads with a 300-cycle kit (Illumina 20012860). Bulk RNA-seq libraries were sequenced on the NextSeq 500 Sequencing System (Illumina) using 75 cycle high-output kit (Illumina 20024906).

### 8.1.7 Data Processing

Sequences were collected from the sequencer and de-multiplexed using bcl2fastq version 2.19.0.316. Reads were aligned using to the mm10plus genome using STAR version 2.5.2b with parameters TK. Gene counts were produced using HTSEQ version 0.6.1p1 with default parameters, except ‘stranded’ was set to ‘false’, and ‘mode’ was set to ‘intersection-nonempty’.

### 8.1.8 Gene Set Enrichment Analysis

Gene counts were log normalized and scaled before generating the .gct files. GSEA with the Hallmarks gene sets was run with standard parameters: 1000 permutations of type phenotype, with no collapsing to gene symbols, and weighted enrichment. Gene sets were considered significantly enriched if FDR<25%.

### 8.1.9 Human Neurosphere Cultures

A human fetal neural stem cell line from University of California Irvine was developed from fetal neural tissue at 18 week gestational age enriched for CD133+ cells. The use of neural progenitor cells as non-hESC stem cells in this study is compliant to Stanford Stem Cell Research Oversight (SCRO) Protocol 194 pre-approved by the Internal Review Board (IRB)/SCRO of the Stanford Research Compliance Office (RCO). Informed consent was obtained, and standard material transfer agreement signed. Cells were grown in nonadherent ultra-low attachment well plates in X-VIVO 15 media (LONZA) supplemented with LIF (10 ng/ml), N2 Supplement, N-acetylcysteine (63 ug/ml), Heparin (2 ug/ml), EGF (20 ng/ml), and FGF (20 ng/ml).

For limiting dilution analysis, cells were directly plated into 96-well ultra-low adherent plates (Corning Costar) in limiting dilutions down to one cell per well. Each plating dose was done in replicates of up to 12 wells in each experiment, and the number of wells with neurospheres was counted after 10 days. Experiment was repeated 3 times.

### 8.1.10 Lentivirus Production

cDNA for mutant APP harboring the Swedish and Indiana mutations was cloned into a pHIV-Zsgreen backbone obtained from Addgene. Lipofectamine 2000 was used to transduce the construct (either pHIV-Zsgreen+mutant APP or pHIV-Zsgreen alone) into H293T cells and media was collected after 48 hours. Virus was ultra-centrifuged and resuspended in PBS then titered before infecting human fetal neurospheres.

### 8.1.11 Colony Counts

Human neurospheres were dissociated into single cells and infected with either a lentiviral construct containing pHIV-Zsgreen+mutant APP or pHIV-Zsgreen alone and allowed to grow for a week. Thereafter, cells were again dissociated and seeded at 5,000 cells/well in a 24-well plate in triplicate. Cells were fed every day with 20× media containing the appropriate amount of LDN-19389 (Selleckchem S2618). Colonies were counted after 7 days.

### 8.1.12 Immunofluorescence

Neurospheres were cytospun onto slides and fixed in ice-cold methanol for 5 minutes. Slides were rinsed 3 times in phosphate-buffered saline (PBS) at room temperature, followed by blocking in 3% BSA in PBS for 1 hour at room temperature. Rabbit antibody to pSMAD 1/5/8 (1:100; CST 9516) and mouse antibody to beta-amyloid (1:100; Invitrogen 13-200) were diluted in the same 3% blocking buffer and incubated overnight at 4° C. The following day, sections were rinsed three times in 1X PBS and incubated in secondary antibody solution Cy-3 donkey anti-rabbit (1:500; Jackson ImmunoResearch) or Cy-3 donkey anti-mouse (1:500; Jackson ImmunoResearch) and 4′,6-diamidino-2-phenylindole (DAPI) (1:10,000) in 3% blocking solution at room temperature for 2 hours. Slides were then washed 3 times at room temperature in 1X PBS and mounted.

### 8.1.13 Mice

Tg-SwDI mice (background C57BI/6) were purchased from Jackson Laboratories. These mice were made hemizygous for experiments after breeding with Cdkn2a−/−(C57BI6 background) or Usp16+/− mice (back-crossed to B6EiC3). Usp16+/− mice were originally ordered from Mutant Mouse Regional Resource Centers (MMRRC) and Cdkn2a−/−(B6.129-Cdkn2atm1Rdp) were obtained from Mouse Models of Human Cancers Consortium (NCI-Frederick). Wild-type littermates were used as control mice. Mice were genotyped by traditional PCR according to animal's provider. Mice were housed in accordance with the guidelines of Institutional Animal Care Use Committee. All animal procedures and behavioral studies involved in this manuscript are compliant to Stanford Administrative Panel on Laboratory Animal Care (APLAC) Protocol 10868 pre-approved by the Stanford Institutional Animal Care and Use Committee (IACUC).

### 8.1.14 Mouse Neurosphere Cultures

To produce neurospheres, mice were euthanized by CO2, decapitated and the brain immediately removed. The subventricular zone was micro-dissected and stored in ice-cold PBS for further processing. The tissue was digested using Liberase DH (Roche) and DNAse I (250 U/ml) at 37° C. for 20 minutes followed by trituration. Digested tissue was washed in ice-cold HBSS without calcium and magnesium, filtered through a 40-μm filter and immediately put into neurosphere growth media that is, Neurobasal-A (Invitrogen) supplemented with Glutamax (Life Technologies), 2% B27-A (Invitrogen), mouse recombinant epidermal growth factor (EGF; 20 ng/ml) and basic fibroblast growth factor (bFGF; 20 ng/ml) (Shenandoah Biotechnology).

For limiting dilution analysis, cells were directly plated into 96-well ultra-low adherent plates (Corning Costar) in limiting dilutions down to one cell per well. Each plating dose was done in replicate of up to 12 wells in each experiment, and the number of wells with neurospheres was counted after 10 days. For passaging, neurospheres were dissociated and re-plated at a density of 10 cells/uL.

### 8.1.15 RNA Expression Analyses (Mouse)

For real-time analyses, cells were collected in Trizol (Invitrogen), and RNA was extracted following the manufacturer's protocol. Complementary DNA was obtained using Superscript III First Strand Synthesis (Invitrogen). Real-time reactions were assembled using Taqman probes (Applied Biosystems) in accordance with the manufacturer's directions. Expression data were normalized by the expression of housekeeping gene ActB (Mm00607939_s1). Probes used in this study: Cdkn2a (Mm_00494449), Bmi1 (Mm03053308_g1).

### 8.1.16 Immunohistochemistry

All animals were anesthetized with avertin and transcardially perfused with 15 ml phosphate-buffered saline (PBS). Brains were postfixed in 4% paraformaldehyde (PFA) overnight at 4° C. before cryoprotection in 30% sucrose. Brains were embedded in optimum cutting temperature (Tissue-Tek) and coronally sectioned at 40 μm using a sliding microtome (Leica, HM450). For immunohistochemistry, sections were stained using the Click-iT EdU cell proliferation kit and protocol (Invitrogen) to expose EdU labeling followed by incubation in blocking solution [3% normal donkey serum, 0.3% Triton X-100 in phosphate-buffered saline (PBS)] at room temperature for 1 hour. Goat antibody to Sox2 (anti-Sox2) (1:50; R&D Systems AF2018) and rabbit anti-GFAP (1:500; Stem Cell Technologies 60128) were diluted in 1% blocking solution (normal donkey serum in 0.3% Triton X-100 in PBS) and incubated overnight at 4° C. Secondary-only stains were performed as negative controls. The following day, sections were rinsed three times in 1X PBS and incubated in secondary antibody solution (1:500) and 4′,6-diamidino-2-phenylindole (DAPI) (1:10,000) in 1% blocking solution at 4° C. for 4 hours. The following secondary antibodies were used: Alexa 594 donkey anti-rabbit (Jackson ImmunoResearch), Alexa 647 donkey anti-goat (Jackson ImmunoResearch). The next day, sections were rinsed three times in PBS and mounted with ProLong Gold Antifade (Cell Signaling) mounting medium. For senile plaques, sections were incubated for 8 min in aqueous 1% Thioflavin S (Sigma) at room temperature, washed in ethanol and mounted. Total plaque area from images taken of 6 sections were analyzed from each mouse with n=3 mice in each group.

### 8.1.17 Confocal Imaging and Quantification

All cell counting was performed by experimenters blinded to the experimental conditions using a Zeiss LSM700 scanning confocal microscope (Carl Zeiss). For EdU stereology, all EdU-labeled cells in every 6th coronal section of the SVZ were counted by blinded experimenters at 40× magnification. The total number of EdU-labeled cells co-labeled with Sox2 and GFAP per SVZ was determined by multiplying the number of EdU+GFAP+Sox2+ cells by 6. Cells were considered triple-labeled when they colocalized within the same plane.

### 8.1.18 Behavioral Testing for Novel Object Recognition

One behavioral test used in this study for assessing long term memory was novel object recognition (NOR) 67 carried out in arenas (50 cm×50 cm×50 cm) resting on an infra-red emitting base. Behavior was recorded by an infrared-sensitive camera placed 2.5 m above the arena. Data were stored and analyzed using Videotrack software from ViewPoint Life Sciences, Inc. (Montreal, Canada) allowing the tracking of body trajectory/speed and the detection of the nose position. On the day before NOR training, the mouse was habituated to the apparatus by freely exploring the open arena. NOR is based on the preference of mice for a novel object versus a familiar object when allowed to explore freely. For NOR training, two identical objects were placed into the arena and the animals were allowed to explore for 10 minutes. Testing occurred 24 hours later in the same arena but one of the familiar objects used during training was replaced by a novel object of similar dimensions, and the animal was allowed to explore freely for 7 min. The objects and the arena were cleaned with 10% ethanol between trials. Exploration of the objects was defined by the time spent with the nose in a 2.5 cm zone around the objects. The preference index (P.I.) was calculated as the ratio of the time spent exploring the novel object over the total time spent exploring the two objects. The P.I. was calculated for each animal and averaged among the groups of mice by genotype. The P.I. should not be significantly different from 50% in the training session, but is significantly different if novelty is detected.

### 8.2 Example 2. Neural Precursor Cell Exhaustion is the Earliest Sign of Disease in Tg-SwDI Mice

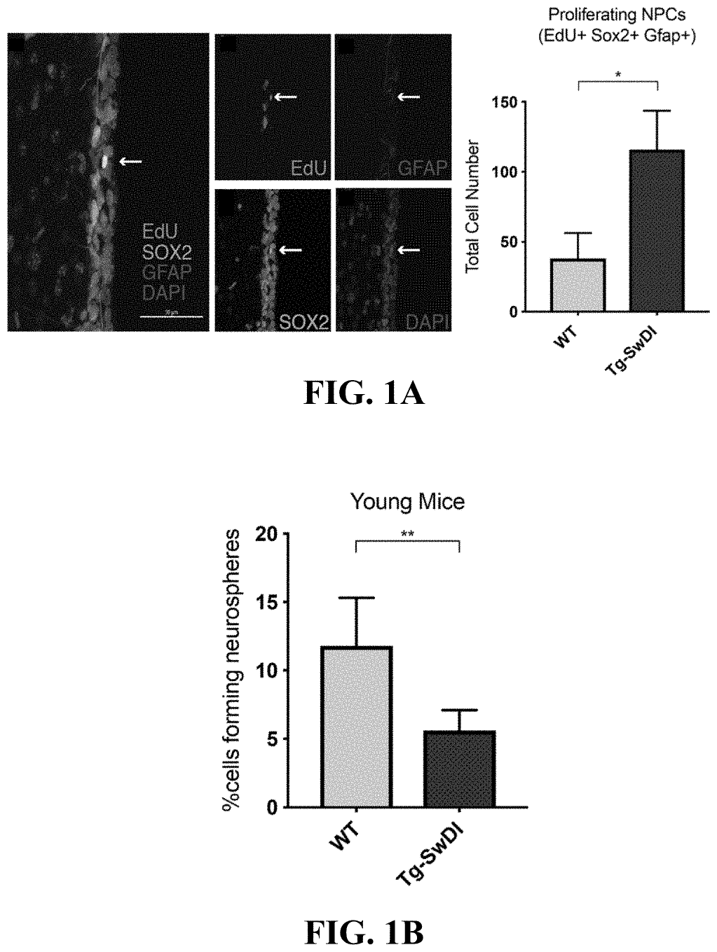

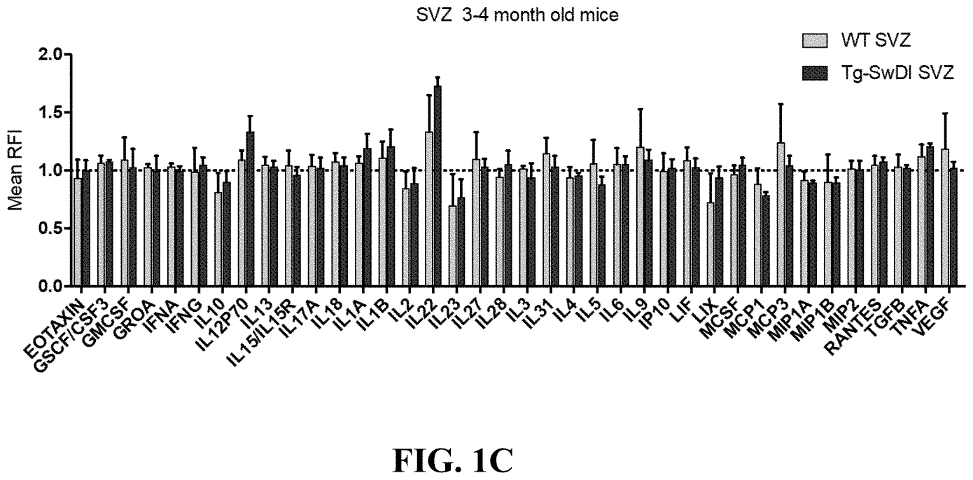

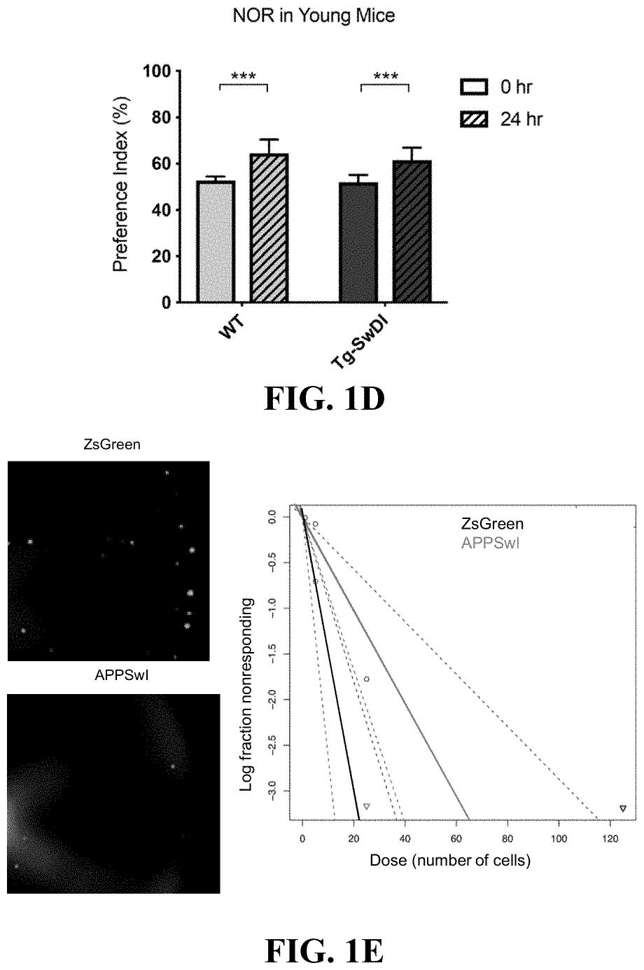

Detecting disease early before fulminant pathogenesis is key to developing effective diagnosis and treatment, particularly when it comes to irreversible degeneration. Therefore, we used a multimodal temporal approach including immunofluorescence staining, in vitro neurosphere assays, Luminex assays, and behavioral studies to dissect changes at the molecular, cellular, and organismal levels in both 3-4 month old and 1 year old AD mice. At 3 months of age, we found that hyperproliferation of neural progenitor cells, marked by 5-ethynyl-2′-deoxyuridine (EdU), (Chehrehasa et al., 2009) SOX2 and GFAP, was increased three-fold in the SVZ of Tg-SwDI mice (P=0.0153; FIG. 1A). In many tissues including the blood, pancreas, intestine and mammary gland, hyperproliferation has been linked to a premature decline in stem cell function associated with aging (Essers et al., 2009; Krishnamurthy et al., 2006; Scheeren et al., 2014). Using extreme limiting dilution analysis (ELDA) of neurosphere-formation from single cells, (Hu and Smyth, 2009; Pastrana et al., 2011) we discovered that 3-4 month old Tg-SwDI mice had significantly less regenerative potential of the SVZ cells than that of healthy age-matched control mice (neurosphere-initiating cell (NIC) frequencies: 1 in 14.5 vs 1 in 7.5, respectively, P=0.00166, FIG. 1B). Table 1, below, summarizes the lower, upper and estimates of 1/NIC for the different genotypes calculated by ELDA.

TABLE 1
Confidence intervals for 1/NIC in young mice

| Age | Group | Lower | Estimate | Upper |
|---|---|---|---|---|
| 3-4 months | WT | 9.82 | 7.5 | 5.75 |
| 3-4 months | Tg-SwDI | 19.74 | 14.5 | 10.68 |

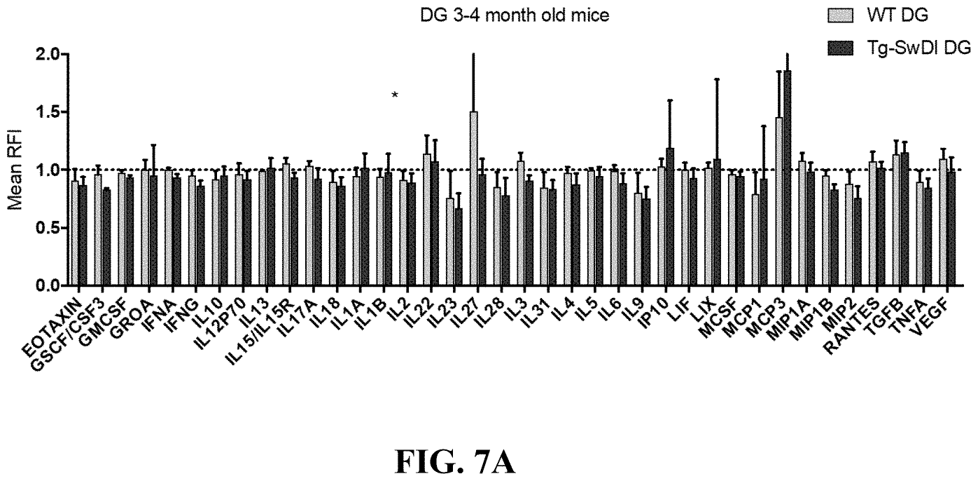

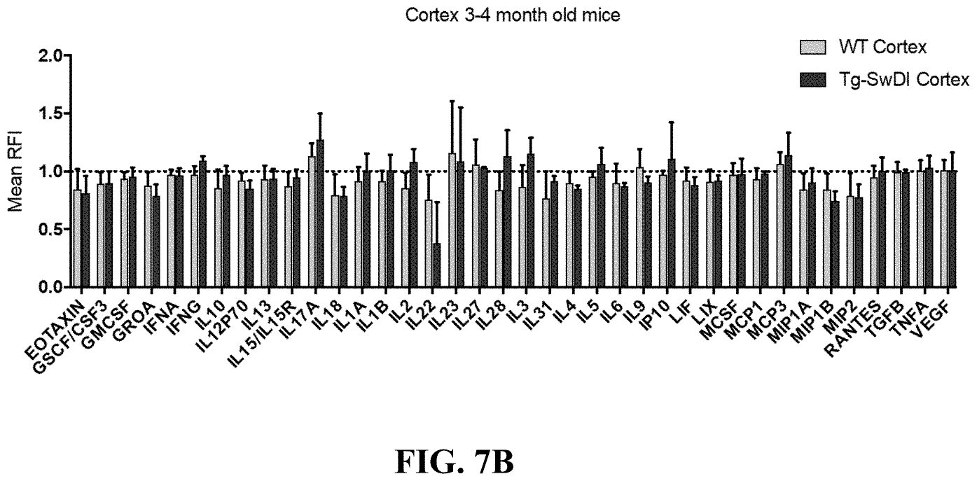

A well-established prominent phenotype associated with AD is inflammation, although it is not clear when brain inflammation can first be detected. To explore whether an inflammatory signature was present by 3-4 months of age, we employed a Luminex screen to assess the presence of an array of cytokines and other inflammatory markers. We looked at the SVZ, hippocampal dentate gyrus, and cortex in 3-4 month old mice, but found no significant differences in inflammatory markers between Tg-SwDI and wild type mice in any of these regions (FIG. 1C and FIGS. 7A, 7B and 7C).

One of the most debilitating features of AD is memory impairment and progressively diminished cognitive function. Although Tg-SwDI mice are known to exhibit these features, there was no evidence of cognitive impairment in 3-4 month old mice when subjected to novel object recognition (NOR) training and subsequent testing after 24 hours (Ennaceur and Delacour, 1988) (FIG. 1D).

Given the only aberrant phenotype in the young Tg-SwDI mouse model was hyperproliferation and self-renewal, we investigated whether or not expression of mutant APP in human NPCs might also cause a self-renewal defect. We therefore infected human fetal neurospheres with a lentiviral construct for either pHIV-Zsgreen alone or pHIV-Zsgreen with Swedish and Indiana APP mutations (APPSwl). Employing the same limiting dilution assay as before, we found diminished NIC frequency of mutant APP-infected human neurospheres compared to cells infected with an empty vector (1 in 19.63 vs 1 in 6.73, respectively, P=5.33e-7; FIG. 1E). This result suggests that the self-renewal defect seen in the Tg-SwDI Alzheimer's model is likely cell-intrinsic and not specific to the Swedish, Dutch and Iowa mutations, but more broadly seen with other APP mutations. Importantly, these results also demonstrate that the effects observed in NPCs derived from a genetic mouse model can be robustly recapitulated in human NPCs expressing mutant APP.

Table 2, below, lists the estimated stem cell frequencies and ranges for each group, calculated using the ELDA software (n=3 separate infections and limiting dilution experiments) (P=5.33e-7). Table 3 summarizes the lower, upper and estimates of 1/NIC for the different genotypes in aged mice calculated by ELDA.

TABLE 2
Confidence intervals for 1/NIC in human fetal NSPs

| Group | Lower | Estimate | Upper |
|---|---|---|---|
| ZsGreen | 5.62 | 4.0 | 2.86 |
| APPSwI | 19.0 | 13.8 | 10.0 |

TABLE 3
Confidence intervals for 1/NIC in aged mice

| Age | Group | Lower | Estimate | Upper |
|---|---|---|---|---|
| 12 months | WT | 24.3 | 17.6 | 12.8 |
| 12 months | Tg-SwDI | 52.8 | 35.5 | 23.8 |

### 8.3 Example 3. Modest Aging in Tg-SwDI Accelerates NPC Exhaustion and Astrogliosis Prior to Inflammation

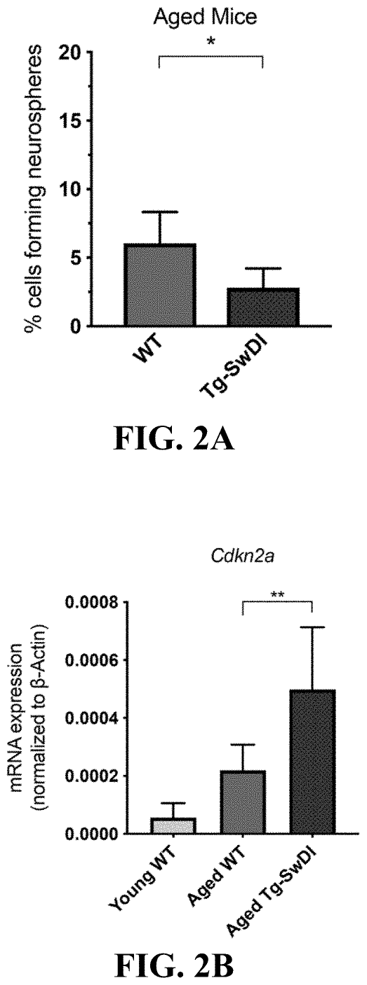

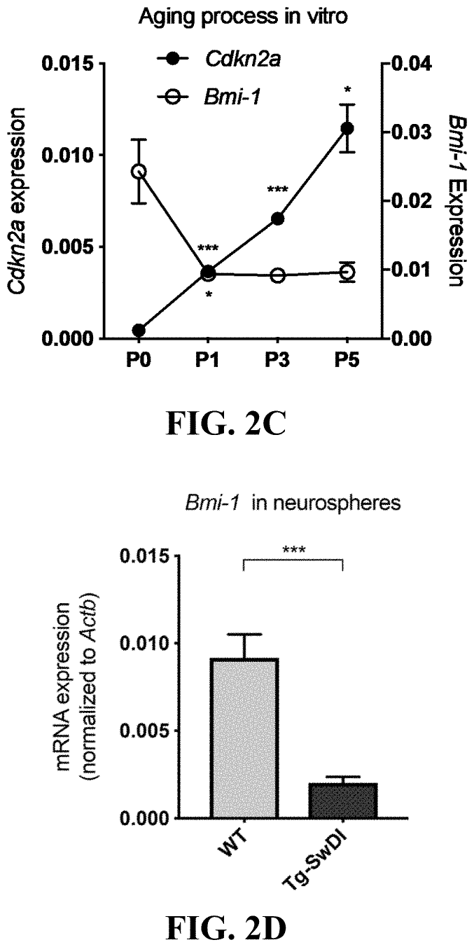

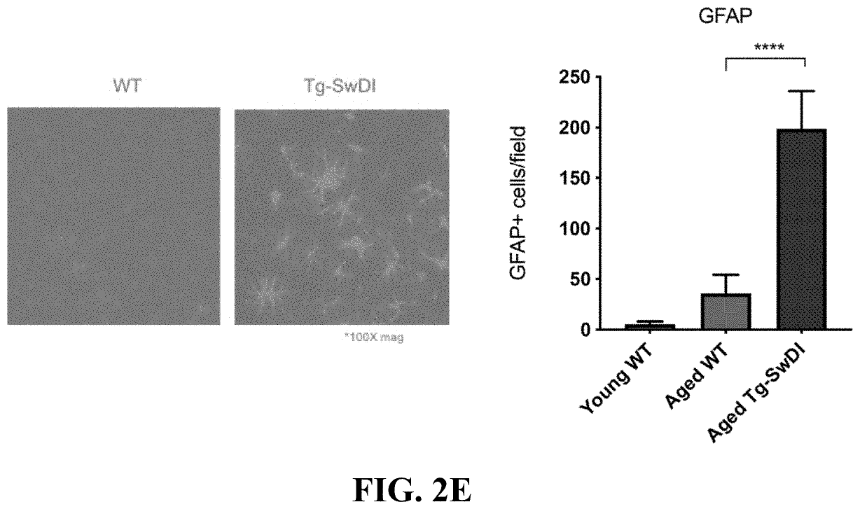

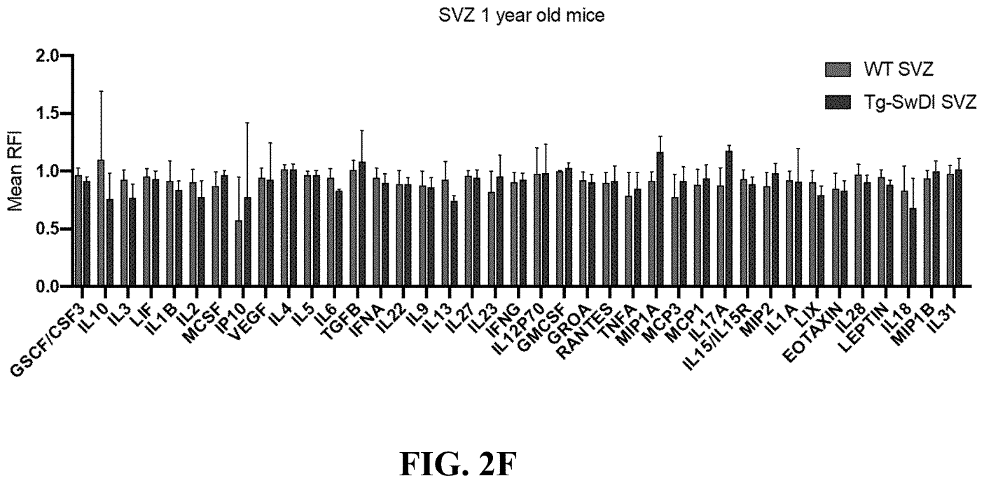

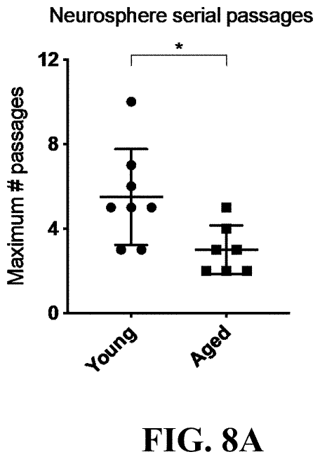

To explore progression of the disease with aging, we next looked at what phenotypic changes occur in 1 year old Tg-SwDI mice. The defect in self-renewal that was observed in the 3-4 month old Tg-SwDI mice was exacerbated in the 1 year old Tg-SwDI mice (1 in 35 for Tg-SwDI mice vs 1 in 17 for WT mice, P=0.00625; FIG. 2A). Furthermore, the NIC capacity in 1 year old controls was similar to that of young Tg-SwDI mice (1 in 17 vs 1 in 14, respectively, compare Tables in FIG. 1b to FIG. 2a), emulating an accelerated aging phenotype with mutant APP. In fact, continuous passaging of neurospheres diminished with age alone in healthy control mice (P=0.021; FIG. 8A), and aging more severely impacted the Tg-SwDI mice. In line with these findings, expression of the well-studied gene, Cdkn2a, known for its increased expression with aging and critical function of inhibiting stem cell self-renewal during development and throughout the lifespan, was increased with aging and even more so in the Tg-SwDI cortex (FIG. 2B).

Reactive astrogliosis, the abnormal increase and activation of astrocytes that can drive degeneration of neurons, has also been linked to both AD disease pathogenesis (Osborn et al., 2016) and to the BMI1/Cdkn2a pathway. Specifically, Zencak and colleagues showed increased astrogliosis in the brain of Bmi1−/− mice (Zencak et al., 2005). As a secondary effect of the reduced passaging capacity of neurospheres during aging, we observed an increase in Cdkn2a expression in neurospheres along with a decrease in Bmi1 expression (FIG. 2C). In neurospheres derived from the SVZ of Tg-SwDI mice, we observed an even greater decrease in Bmi1 expression (FIG. 2D). Furthermore, we observed that astrogliosis, marked by GFAP+ cells, increases with age in 1 year old mice and is further exacerbated in Tg-SwDI mice (P=0.0208, P=0.0010, respectively; FIG. 2E).

Often associated with astrogliosis is neuroinflammation (Frost and Li, 2017). As previously tested in the 3-4 month old mice, we looked for the presence of inflammatory cytokines using the Luminex array in 1 year old mice. We predicted inflammation may explain some of the aging phenotypes observed thus far. However, even at 1 year old, there were no significant differences in the SVZ, DG, or cortex between the WT and Tg-SwDI mice (FIG. 2F and FIG. 8B). Taken together with our data in human NPCs expressing mutant APP, the phenotypes seen at both 3-4 months old and 1 year old suggest a cell intrinsic defect rather than an outcome of cell extrinsic factors like inflammation. Moreover, through these data examining the phenotypic changes occurring with aging in Tg-SwDI mice, we were able to identify an NPC defect as the earliest indication of disease.

### 8.4 Example 4. Self-Renewal Defects are Rescued by Usp16 and Cdkn2a Modulation

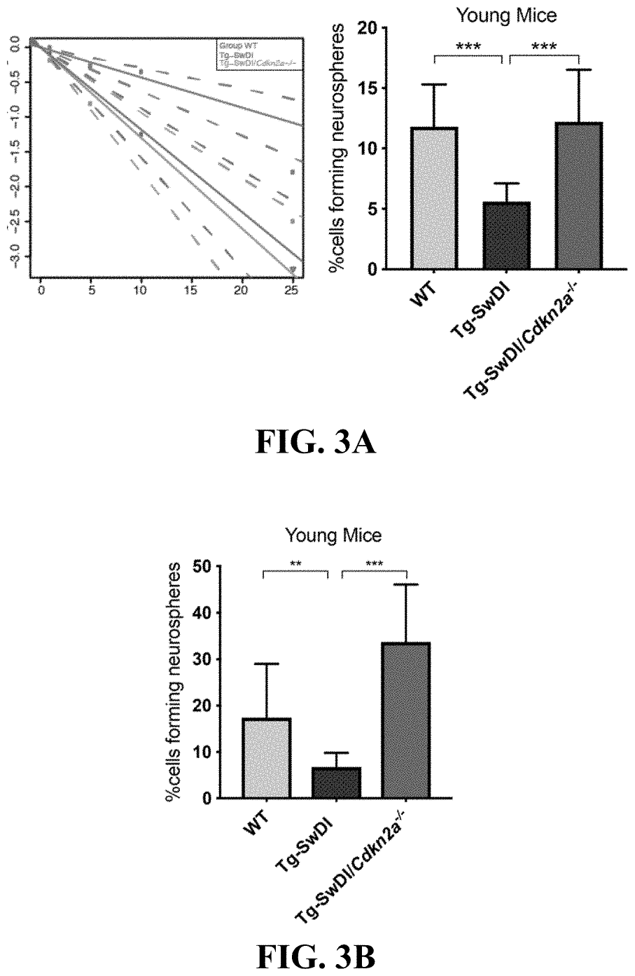

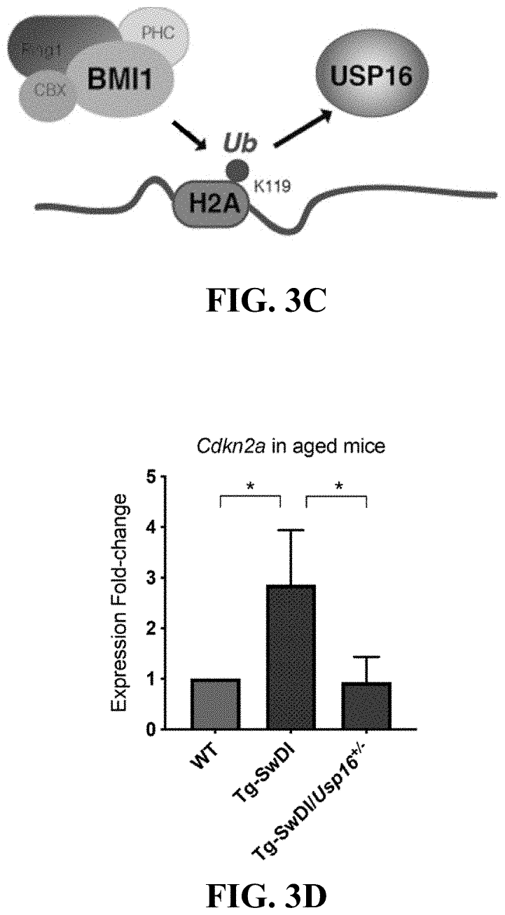

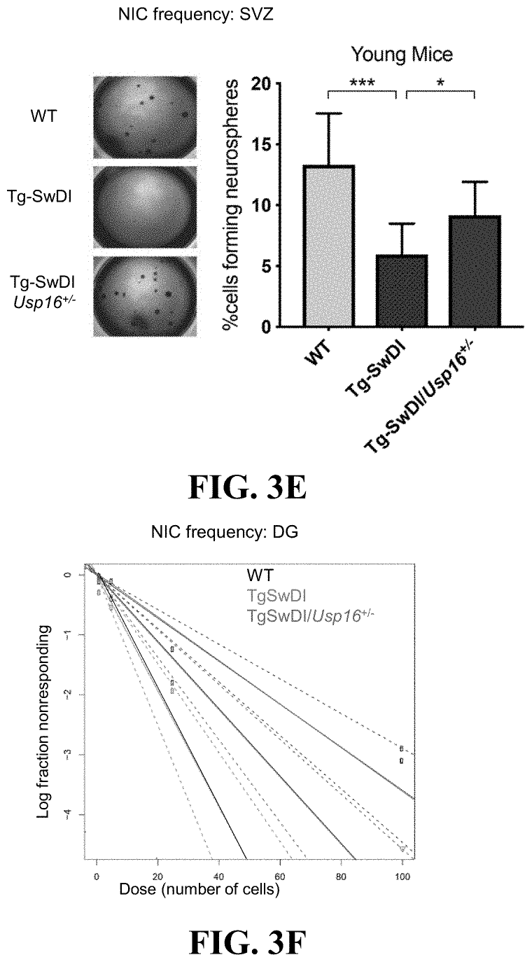

Neural precursor cells function through a number of genetic and epigenetic components, and one of the well described master regulators is Cdkn2a, a gene tightly regulated by BMI1 (Bruggeman et al., 2005). When we crossed the Tg-SwDI mouse with a Cdkn2a knockout mouse (Tg-SwDI/Cdkn2a−/−) and performed limiting dilution assays in SVZ cells from 3-month old mice, a complete restoration of the NIC frequency in the Tg-SwDI/Cdkn2a−/− cells compared to age- matched Tg-SwDI cells was observed (P=7.7e-05; FIG. 3A). This NIC rescue was also observed in hippocampal cells cultured from microdissection of the dentate gyrus (DG) (P=2.09e-9, FIG. 3B). These results demonstrate that impairment of NPC regeneration, as measured by NIC frequencies, is a function of aging that is accelerated by APP mutations and is mediated through Cdkn2a, a known regulator of self-renewal (Molofsky et al., 2003).

Mutations or loss of function in the Cdkn2a gene lead to tumor formation, making it a poor direct target for therapeutics (Hussussian et al., 1994). Upstream of Cdkn2a is USP16, an antagonist of BMI1 and a de-repressor of Cdkn2a that acts through the enzymatic removal of ubiquitin from histone H2A (FIG. 3C) (Adorno et al., 2013; Joo et al., 2007). We predicted that downregulation of Usp16 would increase BMI1 function to counteract the effects of mutant APP similar to what we observed with knockout of Cdkn2a. This is supported by previous data that showed overexpression of USP16 in human-derived neurospheres led to a marked decrease in the formation of secondary neurospheres (Adorno et al., 2013). To test this, we crossed Tg-SwDI with Usp16+/− mice to generate Tg-SwDI/Usp16+/− mice, which do not show tumor formation. We found that Tg-SwDI mice express greater than two-fold more cortical Cdkn2a than both WT and Tg-SwDI/Usp16+/− mice, for which expression levels were very similar (P=0.0403 and P=0.0483, respectively, FIG. 3D). Limiting dilution experiments of cells isolated from the SVZ and DG of the hippocampus showed that Tg-SwDI/Usp16+/− mice had significantly greater NIC frequencies, partially rescuing the mutant APP self-renewal defect (P=0.0492 and P=0.00233, respectively; FIGS. 3E and F). Similar to the NIC rescue in the Tg-SwDI/Cdkn2a−/−, these data show cell-intrinsic impaired self-renewal in the Tg-SwDI model of familial AD, and that reversal of this impairment is possible through targeting Cdkn2a upstream regulator, USP16.

Table 4 shows Confidence intervals for 1/NIC in the dendate gyrus of young mice and Table 5 lists stem cell frequencies and statistics, including p values for each group comparison.

TABLE 4
Confidence intervals for 1/NIC in DG of young mice

| Age | Group | Lower | Estimate | Upper |
|---|---|---|---|---|
| 3-4 months | Tg-SwDI | 34.1 | 27.32 | 21.90 |
| 3-4 months | Tg-SwDI/Usp16+/− | 21.4 | 17.31 | 14.03 |
| 3-4 months | WT | 13.0 | 9.84 | 7.46 |

TABLE 5
Pairwise tests for differences in stem cell frequencies

| Group 1 | Group 2 | Chisq | DF | P value |
|---|---|---|---|---|
| Tg-SwDI | Tg-SwDI/Usp16+/− | 9.27 | 1 | 0.00233 |
| Tg-SwDI | WT | 32.9 | 1 | 9.9e−9 |
| Tg-SwDI/Usp16+/− | WT | 11.2 | 1 | 0.000812 |

### 8.5 Example 5. RNA-Seq Data Reveals Enriched BMP Signaling in Tg-SwDI Mice that is Rescued by Usp16 Haploinsufficiency

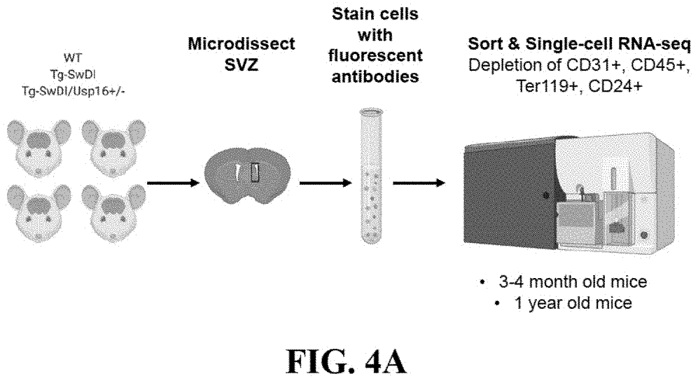

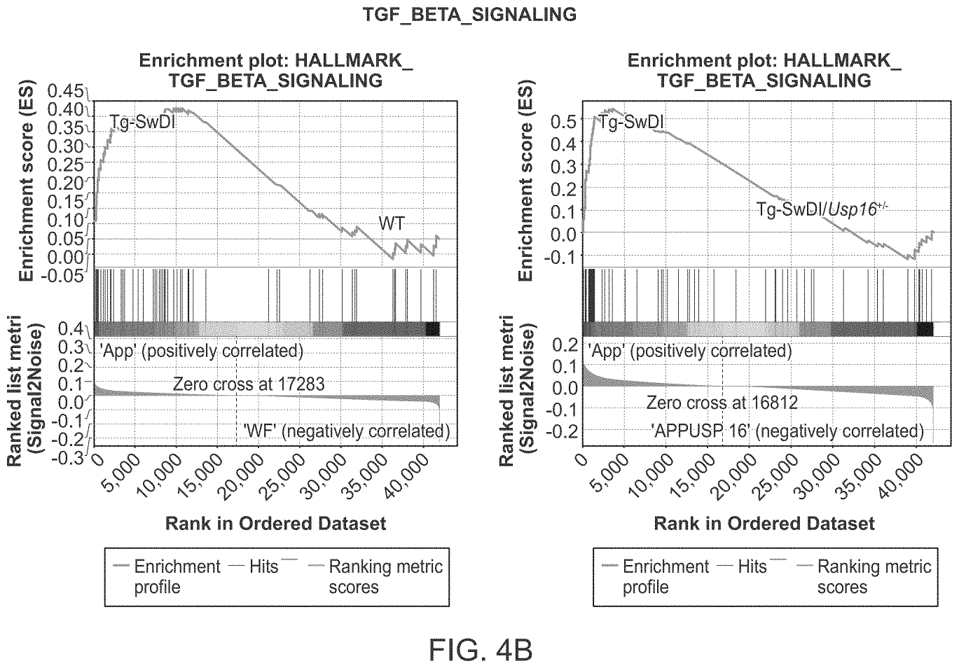

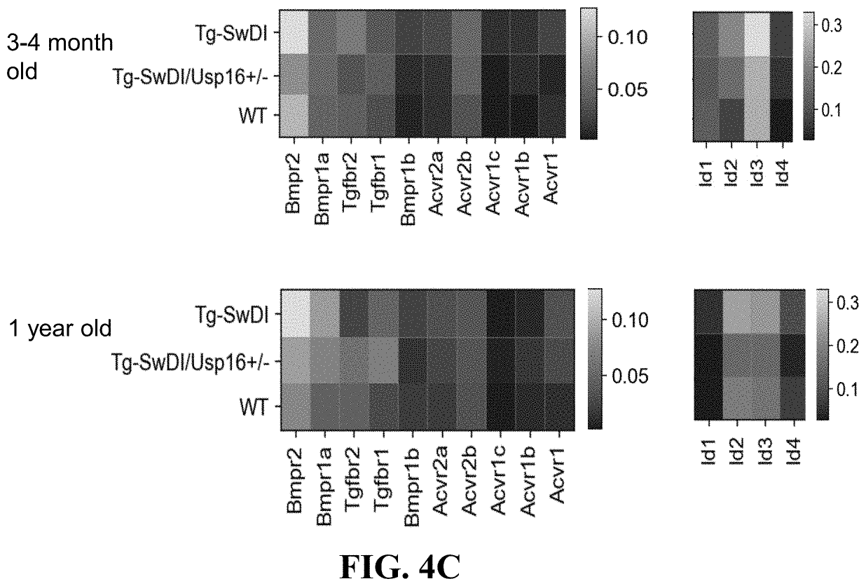

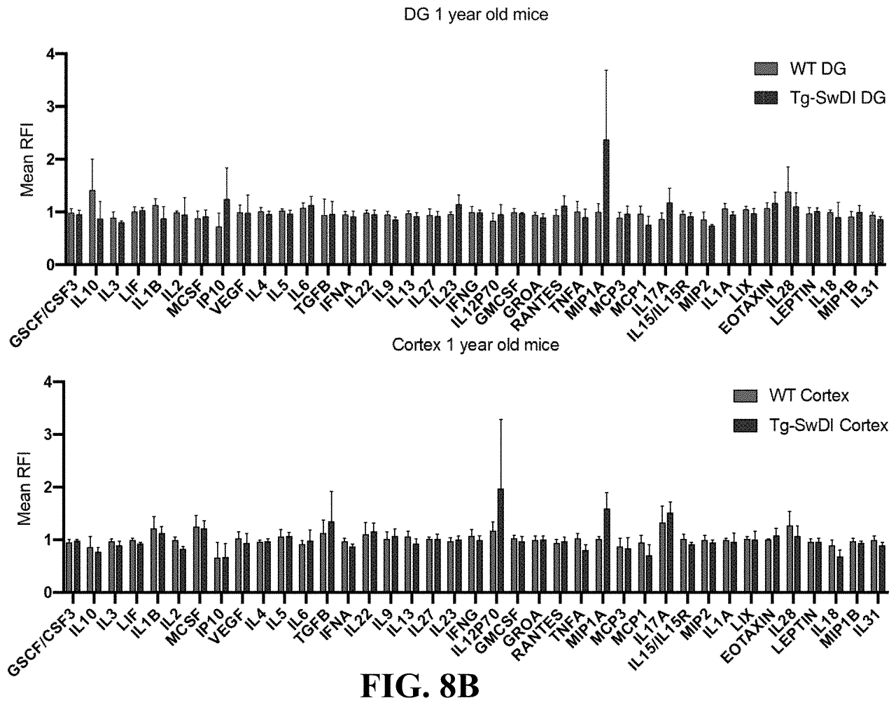

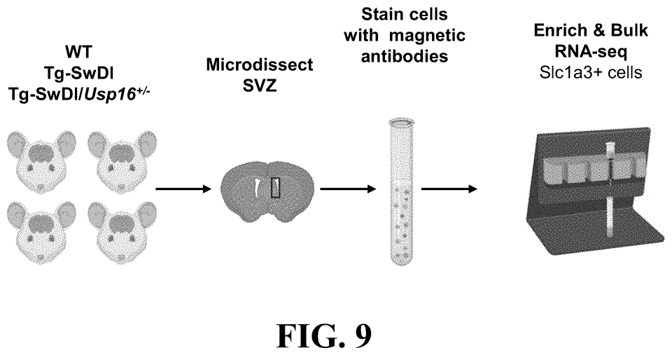

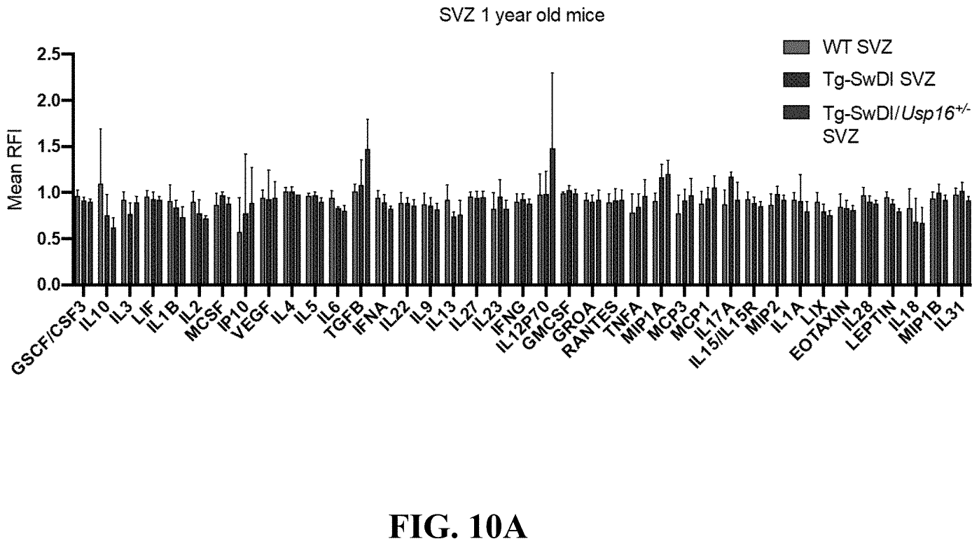

To delineate potential self-renewal pathways that might contribute to the defect and rescue of Tg-SwDI NPCs and Tg-SwDI/Usp/16+/− NPCs, respectively, we performed single-cell RNA-seq and gene set enrichment analysis (GSEA) on primary FACS-sorted CD31-CD45-Ter119-CD24− NPCs from Tg-SwDI, WT, and Tg-SwDI/Usp16+/− mice at 3-4 months and 1 year of age (FIG. 4A) (Mootha et al., 2003; Subramanian et al., 2005). Using the GSEA Hallmark gene sets, we found only three gene sets that were enriched in Tg-SwDI mice over WT mice and rescued in the Tg-SwDI/Usp16+/− mice at both ages: TGF-β pathway, oxidative phosphorylation, and Myc Targets (FIG. 4B). The TGF-β pathway consistently had the highest normalized enrichment score in pairwise comparisons between Tg-SwDI vs WT and Tg-SwDI vs Tg-SwDI/Usp16+/− of the three rescued pathways . We further conducted a bulk RNA-sequencing of GLAST+ enriched NPCs from Tg-SwDI, WT, and Tg-SwDI/Usp16+/− mice at 2 years of age to follow progression of the disease (FIG. 9). Similar to the other time points, TGF-β signaling was enriched in Tg-SwDI NPCs compared to WT and Tg-SwDI/Usp16+/− (Table 7, see below). With further aging, the NPCs also develop an inflammatory signature which was not previously seen at earlier timepoints (Table 8, see below). In looking specifically at the leading-edge genes contributing to the enrichment plots of the TGF-β pathway, we found upregulation of BMP receptors and Id genes which are known to be involved in the BMP pathway, a sub-pathway of the greater TGF-β pathway (FIG. 4C). Heatmaps of average normalized single-cell gene expression showed BMP receptors as the highest expressed TGF-β receptors in the sorted cells, with genes such as BMPR-2, BMPR-1A, Id2, and Id3 upregulated in Tg-SwDI mice and rescued in Tg-SwDI/Usp16+/− mice (FIG. 4D). These data suggest that USP16 may regulate neural precursor cell function specifically through the BMP pathway.

Table 6, below, shows results from GSEA analysis from single-cell RNA-seq data showing pathways enriched in Tg-SwDI mice compared to WT and rescued in Tg-SwDI/Usp16+/− mice. (n=4 for each genotype at each time point; FDR<25%).

TABLE 6
Pathways enriched in Tg-SwDI that are rescued in Tg-SwDI/Usp16+/−
in young and aged mice

| 3-4 months old | 1 year old |
|---|---|
| TGF_BETA_SIGNALING | FATTY_ACID_METABOLISM |
| OXIDATIVE_PHOSPHORYLATION | HEME_METABOLISM |
| MYC_TARGETS_V2 | MTORC1_SIGNALING |
| | CHOLESTEROL_HOMEOSTASIS |
| | ADIPOGENESIS |
| | PROTEIN_SECRETION |
| | PEROXISOME |
| | MYC_TARGETS_V1 |
| | UNFOLDED PROTEIN RESPONSE |
| | WNT_BETA_CATENIN_SIGNALING |
| | PI3K_AKT_MTOR_SIGNALING |
| | MYOGENESIS |
| | XENOBIOTIC_METABOLISM |
| | OXIDATIVE PHOSPHORYLATION |
| | TGF_BETA_SIGNALING |
| | APICAL_JUNCTION |
| | P53_PATHWAY |
| | DNA_REPAIR |
| | UV_RESPONSE_UP |
| | GLYCOLYSIS |
| | BILE_ACID_METABOLISM |
| | IL2_STAT5_SIGNALING |
| | UV_RESPONSE_DN |
| | REACTIVE_OXYGEN_SPECIES |
| | MITOTIC SPINDLE |
| | MYC_TARGETS_V2 |
| | E2F_TARGETS |
| | G2M_CHECKPOINT |
| | HYPOXIA |

Table 7, below, lists normalized enrichment scores of significantly enriched pathways in Tg-SwDI mice compared to WT or Tg-SwDI/Usp16+/− mice at different time points. TGFbeta, Oxidative phosphorylation, and MYC Targets V2 were selected as they were rescued in both 3-4 months and 1 year old mice by Usp16 haploinsufficiency.

TABLE 7
Normalized Enrichment Scores of Significantly
Enriched Pathways in Tg-SwDI mice

| | | Oxidative | MYC |
|---|---|---|---|
| Tg-SwDI vs | TGFbeta | Phosphorylation | Targets V2 |
| WT at 3-4 months | 1.51 | 1.18 | 1.15 |
| Tg-SwDI/Usp16+/− | 1.37 | 1.15 | 1.35 |
| at 3-4 months | | | |
| WT at 1 year old | 1.77 | 1.92 | 1.40 |
| Tg-SwDI/Usp16+/− | 2.30 | 2.02 | 1.59 |
| at 1 year old | | | |
| WT at 2 years old | 1.35 | N/A | N/A |
| Tg-SwDI/Usp16+/− | 1.46 | N/A | N/A |
| at 2 years old | | | |

Table 8 lists specific pathways found to be enriched in Tg-SwDI compared to Tg-SwDI/Usp16+/− and WT mice (n=4 for each genotype at each time point; FDR<25%).

TABLE 8
Pathways Rescued by Usp16 Haploinsufficiency in Tg-SwDI mice
2 year old mice
INFLAMMATORY RESPONSE
INTERFERON ALPHA
INTERFERON GAMMA
TNFA
KRAS_SIGNALING_UP
TGF_BETA_SIGNALING

### 8.6 Example 6. BMPR Inhibition Rescues Stem Cell Defects and Abolishes Increased Phospho-SMAD 1/5/8

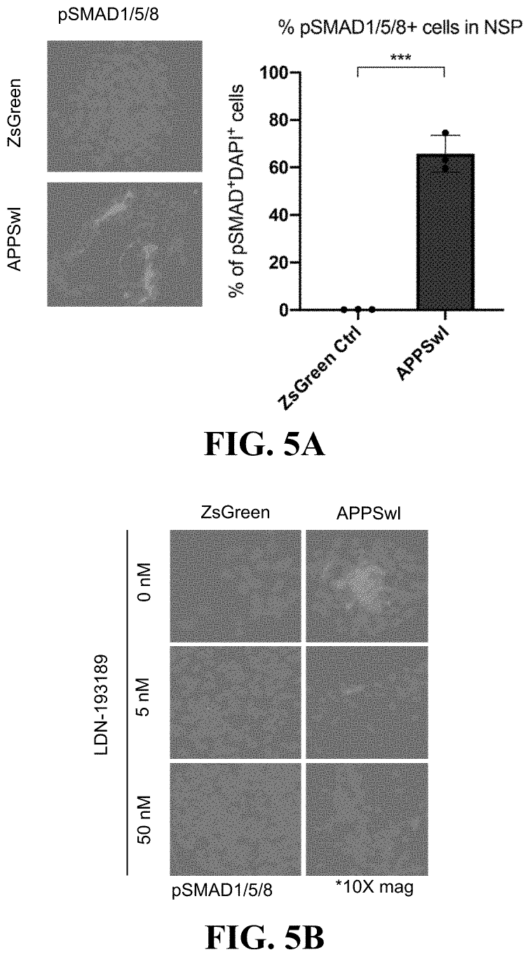

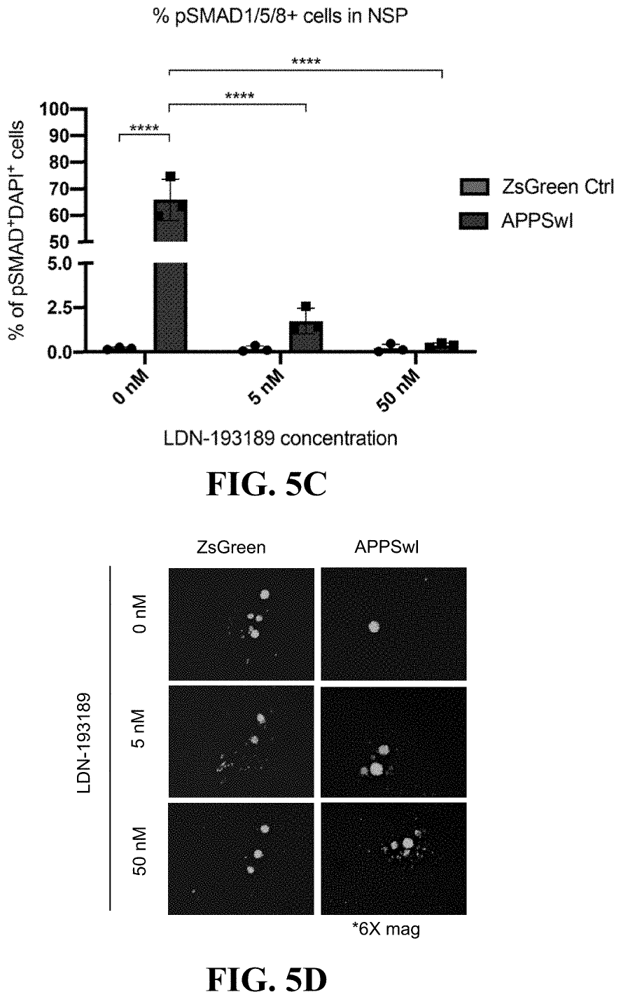

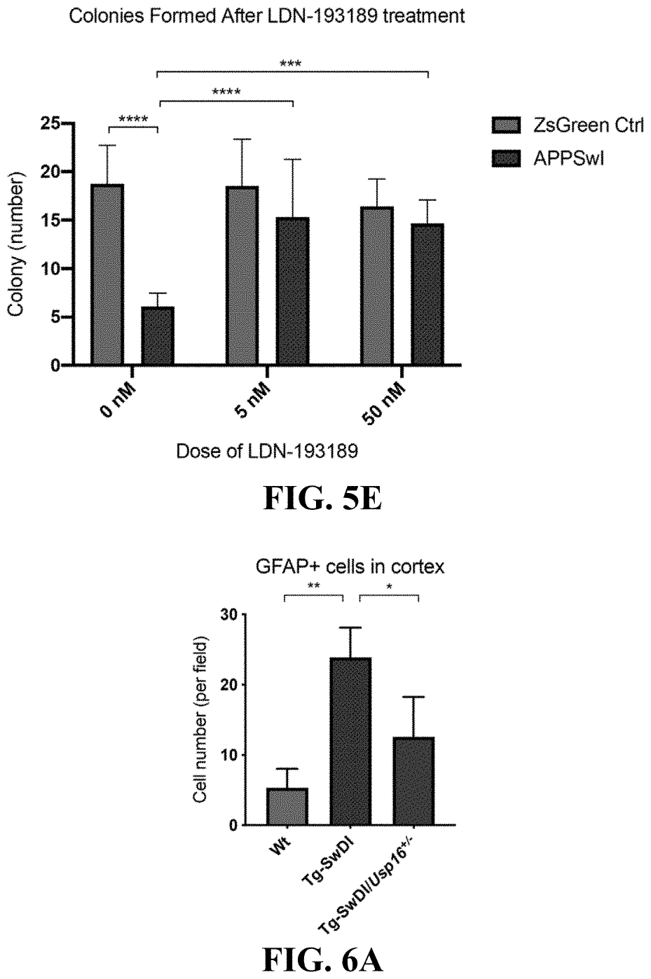

To confirm the functional significance of the BMP pathway in NPC self-renewal, we measured the effects of modulating BMP pathway activity in vitro in human fetal NPCs expressing mutant APP. First, we measured levels of phosphorylated-SMAD (pSMAD) 1, 5, and 8, known readouts of BMP activity, and found they were significantly increased in the mutant neurospheres compared to control (FIG. 5A). Treatment of the neurospheres with the BMP receptor inhibitor LDN-193189, a specific inhibitor of BMP-mediated SMAD1, SMAD5, and SMAD8 activation, substantially decreased pSMAD 1/5/8 (FIGS. 5B and C) (Yu et al., 2008). Furthermore, when we treated neurospheres expressing mutant APP with LDN-193189 for a week, the number of colonies originating from those cells were similar to control cells and significantly higher than untreated mutant APP neurospheres (FIGS. 5D and E). Notably, LDN-193189 had minimal impact on Zsgreen control neurosphere growth (FIG. 5E). This finding demonstrates that the decrease in NIC frequency observed with mutant APP could be explained by the upregulation of BMP signaling. Moreover, BMPR inhibition rescues this defect in cells overexpressing mutant APP at doses that had no toxic effect on healthy cells. Altogether these data reveal that BMP signaling enrichment is recapitulated in human NPCs expressing mutant APP, and that BMP inhibition normalizes the stem cell defect.

### 8.7 Example 7. Astrogliosis is Reduced and Cognitive Function is Restored in Tg-SwDI/Usp16+/− mice

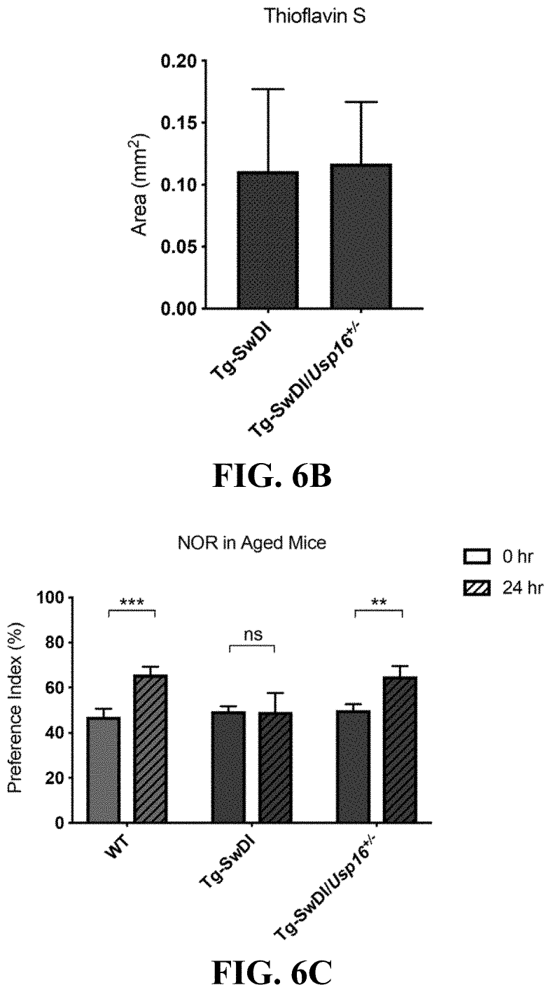

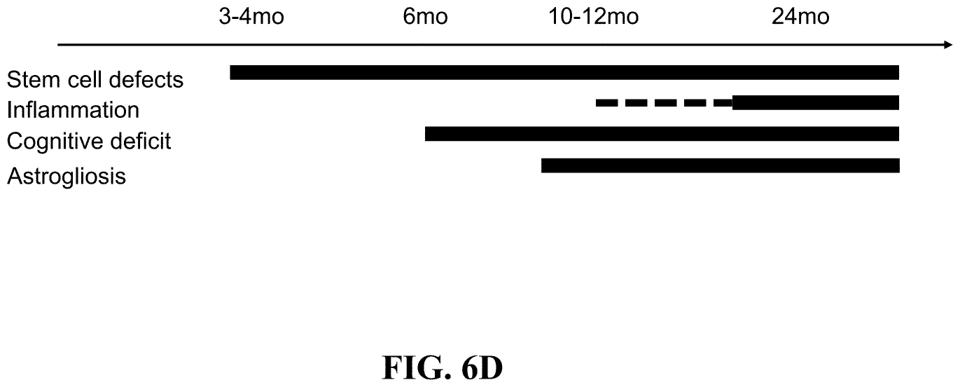

Having identified USP16 as a target to modulate two critical pathways affected by mutations in APP, we investigated its potential effects on downstream pathophysiological markers of AD such as astrogliosis, inflammation, amyloid plaques and memory. Astrogliosis, marked by GFAP+ cells, was increased throughout the cortex in Tg-SwDI mice and was significantly reduced with Usp16 haploinsufficiency (FIG. 6A).

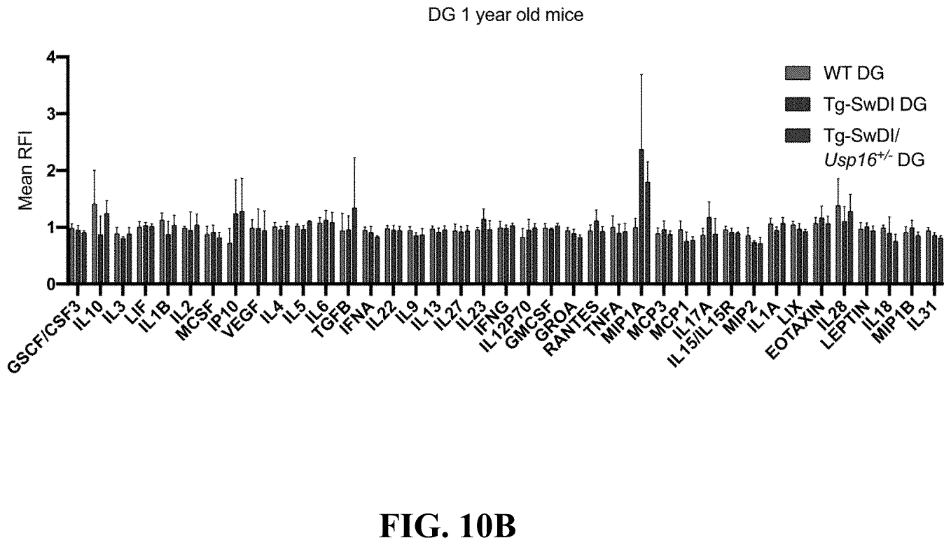

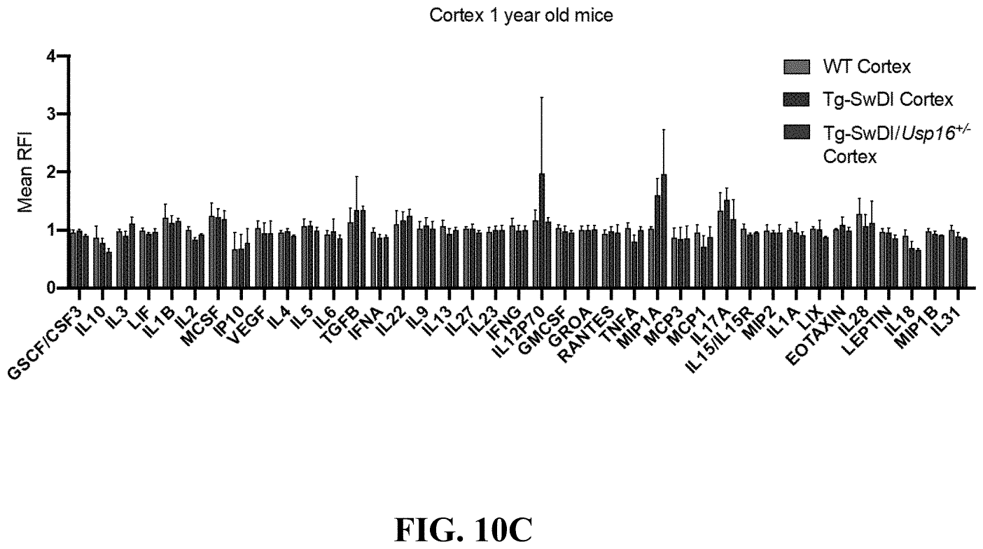

Amyloid plaques are one of the defining features of AD, and controversy exists concerning the effect of plaques on cognitive decline. Mutations in APP lead to amyloid plaque deposition throughout the brain as seen in the aged Tg-SwDI mice. However, no change was observed in plaque burden, demonstrated by Thioflavin S staining, in the age-matched Tg-SwDI/Usp16+/− mice (FIG. 6B). In addition, a Luminex screen of the Tg-SwDI/Usp16+/− mice also did not reveal significant differences in the levels of inflammatory cytokines from any of the groups (FIGS. 10A, 10B and 10C).

As expected, when studying the cognitive decline in the Tg-SwDI cohort, we found that the Tg- SwDI cohort exhibited impaired performance in the NOR task as early as 6 months of age, with preference indexes (P.I.s) that were not significantly different 24 hours after training, indicating no memory of the familiar object (FIG. 6C). The Tg-SwDI/Usp16+/− mice performed equally to their age-matched wild-type controls indicating memory of the familiar object with P.I.s in the 65-70% range (P=0.0099; FIG. 6C). These data indicate that although modulating Usp16 gene dosage does not affect amyloid plaque burden, it ameliorates stem cell self-renewal defects which may be the earliest indication of pathology, as well as reactive astrogliosis and some of the cognitive defects in these mice that occur later (FIG. 6D).

### REFERENCES

1. Abdouh, M., Chatoo, W., El Hajjar, J., David, J., Ferreira, J., and Bernier, G. (2012). Bmi1 is down-regulated in the aging brain and displays antioxidant and protective activities in neurons. PloS one 7, e31870.
2. Adorno, M., Sikandar, S., Mitra, S. S., Kuo, A., Nicolis Di Robilant, B., Haro-Acosta, V., Ouadah, Y., Quarta, M., Rodriguez, J., Qian, D., et al. (2013). Usp16 contributes to somatic stem-cell defects in Down's syndrome. Nature 501, 380-384.
3. Aisen, P. S. (2008). Tarenflurbil: a shot on goal. Lancet Neurol 7, 468-469.
4. Akiyama, H., Barger, S., Barnum, S., Bradt, B., Bauer, J., Cole, G .M., Cooper, N. R., Eikelenboom, P., Emmerling, M., Fiebich, B. L., et al. (2000). Inflammation and Alzheimer's disease. Neurobiol Aging 21, 383-421.
5. Alipour, M., Nabavi, S. M., Arab, L., Vosough, M., Pakdaman, H., Ehsani, E., and Shahpasand, K. (2019). Stem cell therapy in Alzheimer's disease: possible benefits and limiting drawbacks. Mol Biol Rep 46, 1425-1446.
6. Amaya-Montoya, M., Perez-Londono, A., Guatibonza-Garcia, V., Vargas-Villanueva, A., and Mendivil, C. O. (2020). Cellular Senescence as a Therapeutic Target for Age-Related Diseases: A Review. Adv Ther. Baker, D. J., Wijshake, T., Tchkonia, T., LeBrasseur, N. K., Childs, B. G., van de Sluis, B., Kirkland, J. L., and van Deursen, J. M. (2011). Clearance of p16Ink4a-positive senescent cells delays ageing-associated disorders. Nature 479, 232-236.
7. Bateman, R. J., Xiong, C., Benzinger, T. L., Fagan, A. M., Goate, A., Fox, N. C., Marcus, D. S., Cairns, N. J., Xie, X., Blazey, T. M., et al. (2012). Clinical and biomarker changes in dominantly inherited Alzheimer's disease. N Engl J Med 367, 795-804.
8. Bruggeman, S. W., Valk-Lingbeek, M. E., van der Stoop, P. P., Jacobs, J. J., Kieboom, K., Tanger, E., Hulsman, D., Leung, C., Arsenijevic, Y., Marino, S., et al. (2005). Ink4a and Arf differentially affect cell proliferation and neural stem cell self-renewal in Bmi1-deficient mice. Genes Dev 19, 1438-1443.
9. Bussian, T. J., Aziz, A., Meyer, C. F., Swenson, B. L., van Deursen, J. M., and Baker, D. J. (2018). Clearance of senescent glial cells prevents tau-dependent pathology and cognitive decline. Nature 562, 578-582.
10. Castellani, R. J., Rolston, R. K., and Smith, M. A. (2010). Alzheimer disease. Dis Mon 56, 484-546. Chehrehasa, F., Meedeniya, A. C., Dwyer, P., Abrahamsen, G., and Mackay-Sim, A. (2009). EdU, a new thymidine analogue for labelling proliferating cells in the nervous system. J Neurosci Methods 177, 122-130.
11. Doody, R. S., Raman, R., Farlow, M., Iwatsubo, T., Vellas, B., Joffe, S., Kieburtz, K., He, F., Sun, X., Thomas, R. G., et al. (2013). A phase 3 trial of semagacestat for treatment of Alzheimer's disease. N Engl J Med 369, 341-350.
12. Ennaceur, A., and Delacour, J. (1988). A new one-trial test for neurobiological studies of memory in rats. 1: Behavioral data. Behav Brain Res 31, 47-59.
13. Essers, M. A., Offner, S., Blanco-Bose, W. E., Waibler, Z., Kalinke, U., Duchosal, M. A., and Trumpp, A. (2009). IFNalpha activates dormant haematopoietic stem cells in vivo. Nature 458, 904-908.
14. Flamier, A., El Hajjar, J., Adjaye, J., Fernandes, K. J., Abdouh, M., and Bernier, G. (2018). Modeling Late-Onset Sporadic Alzheimer's Disease through BMI1 Deficiency. Cell Rep 23, 2653-2666.
15. Frost, G. R., and Li, Y. M. (2017). The role of astrocytes in amyloid production and Alzheimer's disease. Open Biol 7.
16. Gargiulo, G., Cesaroni, M., Serresi, M., de Vries, N., Hulsman, D., Bruggeman, S. W., Lancini, C., and van Lohuizen, M. (2013). In vivo RNAi screen for BMI1 targets identifies TGF-beta/BMP-ER stress pathways as key regulators of neural- and malignant glioma-stem cell homeostasis. Cancer Cell 23, 660-676.
17. Gjoneska, E., Pfenning, A .R., Mathys, H., Quon, G., Kundaje, A., Tsai, L. H., and Kellis, M. (2015).
18. Conserved epigenomic signals in mice and humans reveal immune basis of Alzheimer's disease. Nature 518, 365-369.
19. Glass, C. K., Saijo, K., Winner, B., Marchetto, M. C., and Gage, F. H. (2010). Mechanisms underlying inflammation in neurodegeneration. Cell 140, 918-934.
20. Green, R. C., Schneider, L. S., Amato, D. A., Beelen, A. P., Wilcock, G., Swabb, E. A., Zavitz, K. H., and
21. Tarenflurbil Phase 3 Study, G. (2009). Effect of tarenflurbil on cognitive decline and activities of daily living in patients with mild Alzheimer disease: a randomized controlled trial. JAMA 302, 2557-2564.
22. Group, A. D. C., Bentham, P., Gray, R., Sellwood, E., Hills, R., Crome, P., and Raftery, J. (2008). Aspirin in Alzheimer's disease (AD2000): a randomised open-label trial. Lancet Neurol 7, 41-49.
23. Haughey, N. J., Liu, D., Nath, A., Borchard, A. C., and Mattson, M. P. (2002). Disruption of neurogenesis in the subventricular zone of adult mice, and in human cortical neuronal precursor cells in culture, by amyloid beta-peptide: implications for the pathogenesis of Alzheimer's disease. Neuromolecular Med 1, 125-135.
24. Hebert, L. E., Weuve, J., Scherr, P. A., and Evans, D. A. (2013). Alzheimer disease in the United States (2010-2050) estimated using the 2010 census. Neurology 80, 1778-1783.
25. Hu, Y., and Smyth, G. K. (2009). ELDA: extreme limiting dilution analysis for comparing depleted and enriched populations in stem cell and other assays. J Immunol Methods 347, 70-78.
26. Huang, Y., and Mucke, L. (2012). Alzheimer mechanisms and therapeutic strategies. Cell 148, 1204-1222.
27. Hunter, S., Arendt, T., and Brayne, C. (2013). The senescence hypothesis of disease progression in Alzheimer disease: an integrated matrix of disease pathways for FAD and SAD. Mol Neurobiol 48, 556-570.
28. Hussussian, C. J., Struewing, J. P., Goldstein, A. M., Higgins, P. A., Ally, D. S., Sheahan, M. D., Clark, W. H., Jr., Tucker, M .A., and Dracopoli, N. C. (1994). Germline p16 mutations in familial melanoma. Nat Genet 8, 15-21.
29. Imbimbo, B. P., Solfrizzi, V., and Panza, F. (2010). Are NSAIDs useful to treat Alzheimer's disease or mild cognitive impairment? Front Aging Neurosci 2.
30. Joo, H. Y., Zhai, L., Yang, C., Nie, S., Erdjument-Bromage, H., Tempst, P., Chang, C., and Wang, H. (2007). Regulation of cell cycle progression and gene expression by H2A deubiquitination. Nature 449, 1068-1072.
31. Krishnamurthy, J., Ramsey, M. R., Ligon, K. L., Torrice, C., Koh, A., Bonner-Weir, S., and Sharpless, N. E. (2006). p16INK4a induces an age-dependent decline in islet regenerative potential. Nature 443, 453-457.
32. Kwak, Y. D., Hendrix, B. J., and Sugaya, K. (2014). Secreted type of amyloid precursor protein induces glial differentiation by stimulating the BMP/Smad signaling pathway. Biochem Biophys Res Commun 447, 394-399.
33. Leeman, D. S., Hebestreit, K., Ruetz, T., Webb, A. E., McKay, A., Pollina, E. A., Dulken, B. W., Zhao, X., Yeo, R. W., Ho, T. T., et al. (2018). Lysosome activation clears aggregates and enhances quiescent neural stem cell activation during aging. Science 359, 1277-1283.
34. Lopez-Toledano, M. A., and Shelanski, M. L. (2004). Neurogenic effect of beta-amyloid peptide in the development of neural stem cells. J Neurosci 24, 5439-5444.
35. Molofsky, A. V., He, S., Bydon, M., Morrison, S. J., and Pardal, R. (2005). Bmi-1 promotes neural stem cell self-renewal and neural development but not mouse growth and survival by repressing the p16Ink4a and p19Arf senescence pathways. Genes Dev 19, 1432-1437.
36. Molofsky, A .V., Pardal, R., Iwashita, T., Park, I. K., Clarke, M. F., and Morrison, S. J. (2003). Bmi-1 dependence distinguishes neural stem cell self-renewal from progenitor proliferation. Nature 425, 962-967.
37. Molofsky, A. V., Slutsky, S. G., Joseph, N. M., He, S., Pardal, R., Krishnamurthy, J., Sharpless, N. E., and Morrison, S. J. (2006). Increasing p16INK4a expression decreases forebrain progenitors and neurogenesis during ageing. Nature 443, 448-452.
38. Mootha, V. K., Lindgren, C .M., Eriksson, K. F., Subramanian, A., Sihag, S., Lehar, J., Puigserver, P., Carlsson, E., Ridderstrale, M., Laurila, E., et al. (2003). PGC-lalpha-responsive genes involved in oxidative phosphorylation are coordinately downregulated in human diabetes. Nat Genet 34, 267-273.
39. Mu, Y., and Gage, F. H. (2011). Adult hippocampal neurogenesis and its role in Alzheimer's disease. Mol Neurodegener 6, 85.
40. Osborn, L. M., Kamphuis, W., Wadman, W. J., and Hol, E. M. (2016). Astrogliosis: An integral player in the pathogenesis of Alzheimer's disease. Prog Neurobiol 144, 121-141.
41. Pastrana, E., Silva-Vargas, V., and Doetsch, F. (2011). Eyes wide open: a critical review of sphere-formation as an assay for stem cells. Cell Stem Cell 8, 486-498.
42. Rodriguez, J. J., Jones, V. C., and Verkhratsky, A. (2009). Impaired cell proliferation in the subventricular zone in an Alzheimer's disease model. Neuroreport 20, 907-912.
43. Rodriguez, J. J., and Verkhratsky, A. (2011). Neurogenesis in Alzheimer's disease. J Anat 219, 78-89.
44. Sakamoto, M., Ieki, N., Miyoshi, G., Mochimaru, D., Miyachi, H., Imura, T., Yamaguchi, M., Fishell, G.,
45. Mori, K., Kageyama, R., et al. (2014). Continuous postnatal neurogenesis contributes to formation of the olfactory bulb neural circuits and flexible olfactory associative learning. J Neurosci 34, 5788-5799.
46. Salloway, S., Sperling, R., Fox, N. C., Blennow, K., Klunk, W., Raskind, M., Sabbagh, M., Honig, L. S., Porsteinsson, A. P., Ferris, S., et al. (2014). Two phase 3 trials of bapineuzumab in mild-to-moderate Alzheimer's disease. N Engl J Med 370, 322-333.
47. Scheeren, F .A., Kuo, A. H., van Weele, L. J., Cai, S., Glykofridis, I., Sikandar, S. S., Zabala, M., Qian, D., Lam, J. S., Johnston, D., et al. (2014). A cell-intrinsic role for TLR2-MYD88 in intestinal and breast epithelia and oncogenesis. Nat Cell Biol 16, 1238-1248.
48. Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550.
49. Wilcock, D. M., Lewis, M. R., Van Nostrand, W. E., Davis, J., Previti, M. L., Gharkholonarehe, N., Vitek, M. P., and Colton, C. A. (2008). Progression of amyloid pathology to Alzheimer's disease pathology in an amyloid precursor protein transgenic mouse model by removal of nitric oxide synthase 2. J Neurosci 28, 1537-1545.
50. Winner, B., Kohl, Z., and Gage, F. H. (2011). Neurodegenerative disease and adult neurogenesis. Eur J. Neurosci 33, 1139-1151.
51. Yousef, H., Morgenthaler, A., Schlesinger, C., Bugaj, L., Conboy, I. M., and Schaffer, D. V. (2015). Age-Associated Increase in BMP Signaling Inhibits Hippocampal Neurogenesis. Stem Cells 33, 1577-1588.
52. Yu, P. B., Deng, D. Y., Lai, C. S., Hong, C. C., Cuny, G .D., Bouxsein, M. L., Hong, D. W., McManus, P. M., Katagiri, T., Sachidanandan, C., et al. (2008). BMP type I receptor inhibition reduces heterotopic [corrected] ossification. Nat Med 14, 1363-1369.
53. Zencak, D., Lingbeek, M., Kostic, C., Tekaya, M., Tanger, E., Hornfeld, D., Jaquet, M., Munier, F. L., Schorderet, D. F., van Lohuizen, M., et al. (2005). Bmi1 loss produces an increase in astroglial cells and a decrease in neural stem cell population and proliferation. J Neurosci 25, 5774-5783.
   All cited patents and nonpatent publications are incorporated herein.

SEQUENCE LISTING

SEQ ID NO.: 1
HOMO SAPIENS BONE MORPHOGENETIC PROTEIN RECEPTOR TYPE 1A (BMPR1A),
MRNA
NCBI Reference Sequence: NM_004329.3
LOCUS NM_004329 6417 bp mRNA linear PRI 24 Oct. 2020
DEFINITION Homo sapiens bone morphogenetic protein receptor type 1A
(BMPR1A),
ORGANISM Homo sapiens
Eukaryota; Metazoa; Chordata; Craniata; Vertebrata; Euteleostomi;
Mammalia; Eutheria; Euarchontoglires; Primates; Haplorrhini;
Catarrhini; Hominidae; Homo.
ORIGIN

| | |
|---|---|
| 1 | aagagtcggc ggcggtggcg gcggccgctg cagagattgg aatccgcctg ccgggcttgg |
| 61 | cgaaggagaa gggaggaggc aggagcgagg agggaggagg gccaagggcg ggcaggaagg |
| 121 | cttaggctcg gcgcgtccgt ccgcgcgcgg cgaagatcgc acggcccgat cgaggggcga |
| 181 | ccgggtcggg gccgctgcac gccaagggcg aaggccgatt cgggccccac ttcgccccgg |
| 241 | cggctcgccg cgcccacccg ctccgcgccg agggctggag gatgcgttcc ctggggtccg |
| 301 | gacttatgaa aatatgcatc agtttaatac tgtcttggaa ttcatgagat ggaagcatag |
| 361 | gtcaaagctg tttggagaaa atcagaagta cagttttatc tagccacatc ttggaggagt |
| 421 | cgtaagaaag cagtgggagt tgaagtcatt gtcaagtgct tgcgatcttt tacaagaaaa |
| 481 | tctcactgaa tgatagtcat ttaaattggt gaagtagcaa gaccaattat taaaggtgac |
| 541 | agtacacagg aaacattaca attgaacaat gcctcagcta tacatttaca tcagattatt |
| 601 | gggagcctat ttgttcatca tttctcgtgt tcaaggacag aatctggata gtatgcttca |
| 661 | tggcactggg atgaaatcag actccgacca gaaaaagtca gaaaatggag taaccttagc |
| 721 | accagaggat accttgcctt ttttaaagtg ctattgctca gggcactgtc cagatgatgc |
| 781 | tattaataac acatgcataa ctaatggaca ttgctttgcc atcatagaag aagatgacca |
| 841 | gggagaaacc acattagctt cagggtgtat gaaatatgaa ggatctgatt ttcagtgcaa |
| 901 | agattctcca aaagcccagc tacgccggac aatagaatgt tgtcggacca atttatgtaa |
| 961 | ccagtatttg caacccacac tgccccctgt tgtcataggt ccgttttttg atggcagcat |
| 1021 | tcgatggctg gttttgctca tttctatggc tgtctgcata attgctatga tcatcttctc |
| 1081 | cagctgcttt tgttacaaac attattgcaa gagcatctca agcagacgtc gttacaatcg |
| 1141 | tgatttggaa caggatgaag catttattcc agttggagaa tcactaaaag accttattga |
| 1201 | ccagtcacaa agttctggta gtgggtctgg actaccttta ttggttcagc gaactattgc |
| 1261 | caaacagatt cagatggtcc ggcaagttgg taaaggccga tatggagaag tatggatggg |
| 1321 | caaatggcgt ggcgaaaaag tggcggtgaa agtattcttt accactgaag aagccagctg |
| 1381 | gtttcgagaa acagaaatct accaaactgt gctaatgcgc catgaaaaca tacttggttt |
| 1441 | catagcggca gacattaaag gtacaggttc ctggactcag ctctatttga ttactgatta |
| 1501 | ccatgaaaat ggatctctct atgacttcct gaaatgtgct acactggaca ccagagccct |
| 1561 | gcttaaattg gcttattcag ctgcctgtgg tctgtgccac ctgcacacag aaatttatgg |
| 1621 | cacccaagga aagcccgcaa ttgctcatcg agacctaaag agcaaaaaca tcctcatcaa |
| 1681 | gaaaaatggg agttgctgca ttgctgacct gggccttgct gttaaattca acagtgacac |
| 1741 | aaatgaagtt gatgtgccct tgaataccag ggtgggcacc aaacgctaca tggctcccga |
| 1801 | agtgctggac gaaagcctga acaaaaacca cttccagccc tacatcatgg ctgacatcta |
| 1861 | cagcttcggc ctaatcattt gggagatggc tcgtcgttgt atcacaggag ggatcgtgga |
| 1921 | agaataccaa ttgccatatt acaacatggt accgagtgat ccgtcatacg aagatatgcg |
| 1981 | tgaggttgtg tgtgtcaaac gtttgcggcc aattgtgtct aatcggtgga acagtgatga |
| 2041 | atgtctacga gcagttttga agctaatgtc agaatgctgg gcccacaatc cagcctccag |
| 2101 | actcacagca ttgagaatta agaagacgct tgccaagatg gttgaatccc aagatgtaaa |
| 2161 | aatctgatgg ttaaaccatc ggaggagaaa ctctagactg caagaactgt ttttacccat |
| 2221 | ggcatgggtg gaattagagt ggaataagga tgttaacttg gttctcagac tctttcttca |
| 2281 | ctacgtgttc acaggctgct aatattaaac ctttcagtac tcttattagg atacaagctg |
| 2341 | ggaacttcta aacacttcat tctttatata tggacagctt tattttaaat gtggtttttg |
| 2401 | atgccttttt ttaagtgggt ttttatgaac tgcatcaaga cttcaatcct gattagtgtc |
| 2461 | tccagtcaag ctctgggtac tgaattgcct gttcataaaa cggtgctttc tgtgaaagcc |
| 2521 | ttaagaagat aaatgagcgc agcagagatg gagaaataga ctttgccttt tacctgagac |
| 2581 | tttcagttcg tttgtattct acctttgtaa aacagcctat agatgatgat gtgtttggga |
| 2641 | tactgcttat tttatgatag tttgtcctgt gtccttagtg atgtgtgtgt gtctccatgc |
| 2701 | acatgcacgc cgggattcct ctgctgccat ttgaattaga agaaaataat ttatatgcat |
| 2761 | gcacaggaag atattggtgg ccggtggttt tgtgctttaa aaatgcaata tctgaccaag |
| 2821 | attcgccaat ctcatacaag ccatttactt tgcaagtgag atagcttccc caccagcttt |
| 2881 | attttttaac atgaaagctg atgccaaggc caaaagaagt ttaaagcatc tgtaaatttg |
| 2941 | gactgttttc cttcaaccac catttttttt gtggttatta tttttgtcac ggaaagcatc |
| 3001 | ctctccaaag ttggagcttc tattgccatg aaccatgctt acaaagaaag cacttcttat |
| 3061 | tgaagtgaat tcctgcattt gatagcaatg taagtgccta taaccatgtt ctatattctt |
| 3121 | tattctcagt aacttttaaa agggaagtta tttatatttt gtgtataatg tgctttattt |
| 3181 | gcaaatcacc cactccttta caaccatact ttatatatgt acatacattc atactgtaga |
| 3241 | aaccagctca tgtgtacctc atatcccatc cttaagagaa gaaatgttat aaagtagaac |
| 3301 | taaatataaa ttttcagaat taatgcattc aaagtaatat atcaaatcca ggactttgtt |
| 3361 | aacttcaggt aaaaacttca ttagggtaat atcatctcaa ttttttcaaa tgaaaggatt |
| 3421 | ctctaattag aaatttatat gtcagagctg ttataaattt atcaactgtc aaatatgttc |
| 3481 | tggacagcta aatcatttga gatttttggt tttttgattt ctattcccta acttgtgaag |
| 3541 | acaatgaaaa atcaggcaga aatatttagt atctagtcag tatctgtagc tacactgtat |
| 3601 | aactgttctt caataaaatg gttcatattt tatagatgcc ttgttatctc aagaaatctg |
| 3661 | atttacataa acttatactt ctttaatgct ttttaaatat ttattctgag caaacaattc |
| 3721 | atgagtacat caagtgagat agttttattt gattataaca taaaataaat gtgattatat |
| 3781 | cacatcatca tcaaaaaggt ttaaattaaa tgggaggaaa tcagcatatg tccacccatt |
| 3841 | accaaaattt gactatcatt taaggttaaa acttacaaat ttgtctgcac atcaaatttc |
| 3901 | acaaatttga aaattgcctt aaccattttg attaataagt ttcatctgcc ataattaaag |
| 3961 | tctgaagtgt tcatcaagat aagtaaattt gcatatggat aatacccaat aacttgtttt |
| 4021 | ttcagaattt ttcaccatat gtatactgag aaatacaaat attttaatct gcgttgccgt |
| 4081 | atgatatgat tgcacttaga acacccaatt tacttaaatc ttggtttact tttgacttga |
| 4141 | taccataatc tttaaaatca tttgtcatct tttttttttt ttttttgaga cggagtctcg |
| 4201 | ctctgtcgcc caggctggac tgcggactgc agtggcgcaa tctcggctca ctgcaagctc |
| 4261 | cgcttcccgg gttcacgcca ttctcctgcc tcagcctccc gagtagctgg gactacaggc |
| 4321 | gcccgccacc gcgcccggct aattttttgt atttttagta gagacggggt ttcaccttgt |
| 4381 | tagccaggat ggtctcaatc tcctgacctc atgatccacc tgcctcggcc tcccaaagtt |
| 4441 | catttgtcat cttaataaaa atataaagac aggcaaagtt tattggaaat gttcaaatgg |
| 4501 | tgtgtggaag caaaaaatta cagccagtat atgagaccac tattatggtt ttttaaaatt |
| 4561 | aacttggtct agtaaaagtg atatcaagag ttaatcttag aaacttgctc agtaaaaaca |
| 4621 | ttttctagta taacatgttc tttaaaaagc aaatgctgcc gtctttggaa tcttaatcta |
| 4681 | aaaatgtggc cgggcgcggt ggctcacgcc tgtaatccca acactttggg aggctgaggc |
| 4741 | gggtggatca caaggtcagg agttcaagac cagcctggcc aacatggtga aaccccatct |
| 4801 | ctactaaaaa taaaaaactc agccaggcgt ggtggcgggt gcctgtaatc ccagctactc |
| 4861 | gggaggctga agcaggagaa ttgcttaaaa tcagaaggtg gaggttgcag tgagctgaga |
| 4921 | tcgtgtcact gcactccagc ctgggcaaaa gagcgaaact ccatctcaaa taaacaaaca |
| 4981 | aataaataac aaaaaacaaa aatgttgcat taaacttagt tcttgtctct cctttccact |
| 5041 | cttattctta aatctgaagc tcatcgacta agtgaaatat ttaaagaata tgataggcca |
| 5101 | gcaagaagaa gtattatgta gtaccatagt tagtaaattc gtaaaacctt ggaagccatt |
| 5161 | atttggtccc acttgcaatt tagtgttttt gaagtgtgta gcttcattca gatagctctt |
| 5221 | taaataatta aaatataaaa gcaaacaacc caaactacct gactataaac aggaaaagtt |
| 5281 | aaccctcaaa gagagttctt gtgaattctc tttatgctgg caaatagctc taggattaaa |
| 5341 | ggcacattag ggtttccttc agtttgttta ttctaagctt ttactgtgct ttttactgaa |
| 5401 | caagtttctg atgtataaaa cttgcatctg atttctttgg aaatattttc acaaaagtta |
| 5461 | ttttaatcag tatttttaca ttgcctttcc agtgtccaga agtgtttcta aacttagaaa |
| 5521 | gtgacctata gttttttaaa attatgtttt cctagaacgt gccaaatttt gatttactct |
| 5581 | aacaatcagt acttttcttc agatgctttg ttctgtttag aacaaaaatg cactatagtt |
| 5641 | tttaaagaat catgcatctt tgggttggcc caggatcaaa tttgatattg aataatttat |
| 5701 | tccagggcag ctttcataaa catacttcat agatgttgtt ttgaaatgtt tctaaatatc |
| 5761 | taaaatcatt tcaacagcag aaatgatttt tattttaaca aaagattatg atagcccttg |
| 5821 | tagtgtttaa aagtggtcat atttattact gactttgagt caggtgttaa aatagcagtg |
| 5881 | ccacagctcg tctcttgcct tagtgtgctg ctgtgagagt cacagtggaa actgcaggga |
| 5941 | ggaggtgtgt tcctaagaac caaaatccag cacagcatcc tgtgaagcca cgtgtaatga |
| 6001 | tggtcccata aggaaagtat gtgaatatgg ctcttgtaaa ggattaacta ttgtaatttt |
| 6061 | agcttatgct ctgtattctg ttttctatgg aattatttaa gcccttttag tgacctttgt |
| 6121 | cctggcccat ttaaaaacta aaatgtagta tatattgtat aaaatggaaa tatcattatt |
| 6181 | gcttcattag gggaaactgt acataggcat tgaaagaagg gtaaaagcaa gcagttttat |
| 6241 | caggcagttg taaaacacca aaaatataga ttcgtctttg acgtgtaaca cactaaatgt |
| 6301 | attttgtaca gcatctggtt taaaaggtgc cttaagagtt taccattact tgctttgttc |
| 6361 | tatatacaga ttatgtccaa tgtatcattt tgaagtaaat aaccttattt tagtata |

SEQ. ID NO.: 2
Homo sapiens bone morphogenetic protein receptor type 2 (BMPR2), mRNA
NCBI Reference Sequence: NM_001204.7
LOCUS NM_001204 12068 bp mRNA linear PRI 01 Nov. 2020
DEFINITION Homo sapiens bone morphogenetic protein receptor type 2 (BMPR2),
mRNA.
ORGANISM Homo sapiens
Eukaryota; Metazoa; Chordata; Craniata; Vertebrata; Euteleostomi;
Mammalia; Eutheria; Euarchontoglires; Primates; Haplorrhini;
Catarrhini; Hominidae; Homo.

| | |
|---|---|
| 1 | gactcccccc tttgtgtctg gtctgctcgg agccactgga agtgcctccc ggagggacgc |
| 61 | agggtgtctc gccgcctccc tgcccacccc cttccccggc taccttcatc cgccctcccg |
| 121 | ccgccccccg ccctcggtcc gcgacgcccg agttccgtca ggagcccaga gctgcgggag |
| 181 | aacgaggcgg cggcggcggc ggcggcggcg gcggcggcgg cagcagcagc ggcttcctcg |
| 241 | gggggttgtg attcgctcac aggagccatt gacgggagaa gaggaggctt tcttggtgga |
| 301 | atttacctca ggcaagatcg agccgcagga ataaaaagcg aggaagggaa gggagcgccg |
| 361 | ccgggaggac tagaaggggc agcctctcac acccactccg cctgccgtct cggggagccc |
| 421 | ggaccggggc cgcgaccgcg acccctcccc tcccccgctc ctacctctcc tcagccttcg |
| 481 | ccagggcctc cccaaccctc tcacggttgt tctgcgaagg cgtggggact gtgagcttgt |
| 541 | ccatggaggc aggcaccttt tttgatccag tcaaggaaga ggatttgttg ttttcgaaat |
| 601 | cagagtgaag gaagcaccga agcgaaactt aaggaatcct gccttcccgg agccgcgggc |
| 661 | gatgcgacta gggctgccgg gcgccgccgc cgcccgtccg gcttcgtcct tcccggcagt |
| 721 | cgggaactag ttctgaccct cgccccccga ccccggatcg aatccccgcc ctccgcaccc |
| 781 | tggatatgtt ttctcccaga cctggatatt tttttgatat cgtgaaacta cgagggaaat |
| 841 | aatttggggg atttcttctt ggctccctgc tttccccaca gacatgcctt ccgtttggag |
| 901 | ggccgcggca ccccgtccga ggcgaaggaa cccccccagc cgcgagggag agaaatgaag |
| 961 | ggaatttctg cagcggcatg aaagctctgc agctaggtcc tctcatcagc catttgtcct |
| 1021 | ttcaaactgt attgtgatac gggcaggatc agtccacggg agagaagacg agcctcccgg |
| 1081 | ctgtttctcc gccggtctac ttcccatatt tcttttcttt gccctcctga ttcttggctg |
| 1141 | gcccagggat gacttcctcg ctgcagcggc cctggcgggt gccctggcta ccatggacca |
| 1201 | tcctgctggt cagcactgcg gctgcttcgc agaatcaaga acggctatgt gcgtttaaag |
| 1261 | atccgtatca gcaagacctt gggataggtg agagtagaat ctctcatgaa aatgggacaa |
| 1321 | tattatgctc gaaaggtagc acctgctatg gcctttggga gaaatcaaaa ggggacataa |
| 1381 | atcttgtaaa acaaggatgt tggtctcaca ttggagatcc ccaagagtgt cactatgaag |
| 1441 | aatgtgtagt aactaccact cctccctcaa ttcagaatgg aacataccgt ttctgctgtt |
| 1501 | gtagcacaga tttatgtaat gtcaacttta ctgagaattt tccacctcct gacacaacac |
| 1561 | cactcagtcc acctcattca tttaaccgag atgagacaat aatcattgct ttggcatcag |
| 1621 | tctctgtatt agctgttttg atagttgcct tatgctttgg atacagaatg ttgacaggag |
| 1681 | accgtaaaca aggtcttcac agtatgaaca tgatggaggc agcagcatcc gaaccctctc |
| 1741 | ttgatctaga taatctgaaa ctgttggagc tgattggccg aggtcgatat ggagcagtat |
| 1801 | ataaaggctc cttggatgag cgtccagttg ctgtaaaagt gttttccttt gcaaaccgtc |
| 1861 | agaattttat caacgaaaag aacatttaca gagtgccttt gatggaacat gacaacattg |
| 1921 | cccgctttat agttggagat gagagagtca ctgcagatgg acgcatggaa tatttgcttg |
| 1981 | tgatggagta ctatcccaat ggatctttat gcaagtattt aagtctccac acaagtgact |
| 2041 | gggtaagctc ttgccgtctt gctcattctg ttactagagg actggcttat cttcacacag |
| 2101 | aattaccacg aggagatcat tataaacctg caatttccca tcgagattta aacagcagaa |
| 2161 | atgtcctagt gaaaaatgat ggaacctgtg ttattagtga ctttggactg tccatgaggc |
| 2221 | tgactggaaa tagactggtg cgcccagggg aggaagataa tgcagccata agcgaggttg |
| 2281 | gcactatcag atatatggca ccagaagtgc tagaaggagc tgtgaacttg agggactgtg |
| 2341 | aatcagcttt gaaacaagta gacatgtatg ctcttggact aatctattgg gagatattta |
| 2401 | tgagatgtac agacctcttc ccaggggaat ccgtaccaga gtaccagatg gcttttcaga |
| 2461 | cagaggttgg aaaccatccc acttttgagg atatgcaggt tctcgtgtct agggaaaaac |
| 2521 | agagacccaa gttcccagaa gcctggaaag aaaatagcct ggcagtgagg tcactcaagg |
| 2581 | agacaatcga agactgttgg gaccaggatg cagaggctcg gcttactgca cagtgtgctg |
| 2641 | aggaaaggat ggctgaactt atgatgattt gggaaagaaa caaatctgtg agcccaacag |
| 2701 | tcaatccaat gtctactgct atgcagaatg aacgcaacct gtcacataat aggcgtgtgc |
| 2761 | caaaaattgg tccttatcca gattattctt cctcctcata cattgaagac tctatccatc |
| 2821 | atactgacag catcgtgaag aatatttcct ctgagcattc tatgtccagc acacctttga |
| 2881 | ctatagggga aaaaaaccga aattcaatta actatgaacg acagcaagca caagctcgaa |
| 2941 | tccccagccc tgaaacaagt gtcaccagcc tctccaccaa cacaacaacc acaaacacca |
| 3001 | caggactcac gccaagtact ggcatgacta ctatatctga gatgccatac ccagatgaaa |
| 3061 | caaatctgca taccacaaat gttgcacagt caattgggcc aacccctgtc tgcttacagc |
| 3121 | tgacagaaga agacttggaa accaacaagc tagacccaaa agaagttgat aagaacctca |
| 3181 | aggaaagctc tgatgagaat ctcatggagc actctcttaa acagttcagt ggcccagacc |
| 3241 | cactgagcag tactagttct agcttgcttt acccactcat aaaacttgca gtagaagcaa |
| 3301 | ctggacagca ggacttcaca cagactgcaa atggccaagc atgtttgatt cctgatgttc |
| 3361 | tgcctactca gatctatcct ctccccaagc agcagaacct tcccaagaga cctactagtt |
| 3421 | tgcctttgaa caccaaaaat tcaacaaaag agccccggct aaaatttggc agcaagcaca |
| 3481 | aatcaaactt gaaacaagtc gaaactggag ttgccaagat gaatacaatc aatgcagcag |
| 3541 | aacctcatgt ggtgacagtc accatgaatg gtgtggcagg tagaaaccac agtgttaact |
| 3601 | cccatgctgc cacaacccaa tatgccaatg ggacagtact atctggccaa acaaccaaca |
| 3661 | tagtgacaca tagggcccaa gaaatgttgc agaatcagtt tattggtgag gacacccggc |
| 3721 | tgaatattaa ttccagtcct gatgagcatg agcctttact gagacgagag caacaagctg |
| 3781 | gccatgatga aggtgttctg gatcgtcttg tggacaggag ggaacggcca ctagaaggtg |
| 3841 | gccgaactaa ttccaataac aacaacagca atccatgttc agaacaagat gttcttgcac |
| 3901 | agggtgttcc aagcacagca gcagatcctg ggccatcaaa gcccagaaga gcacagaggc |
| 3961 | ctaattctct ggatctttca gccacaaatg tcctggatgg cagcagtata cagataggtg |
| 4021 | agtcaacaca agatggcaaa tcaggatcag gtgaaaagat caagaaacgt gtgaaaactc |
| 4081 | cctattctct taagcggtgg cgcccctcca cctgggtcat ctccactgaa tcgctggact |
| 4141 | gtgaagtcaa caataatggc agtaacaggg cagttcattc caaatccagc actgctgttt |
| 4201 | accttgcaga aggaggcact gctacaacca tggtgtctaa agatatagga atgaactgtc |
| 4261 | tgtgaaatgt tttcaagcct atggagtgaa attatttttt gcatcattta aacatgcaga |
| 4321 | agatgtttaa aaataaaaaa aaaactgctt tatcctcctg tcagcacccc ctcccacccc |
| 4381 | tgcaacaaag acttgcttta aatagatttc agctatgcag aaaaatttag cttatgcttc |
| 4441 | catattttta aattttgttt tttaagtttt gcacttttgt ttagtctcgc taaagttata |
| 4501 | tttgtctgtt atgaccacag agttatatgt gtgtgtatca aaagtggtct caaaatattt |
| 4561 | ttttaagaaa aaaagcaaaa acaatgtatt gctgataatc agtttggacc agtttcttaa |
| 4621 | ggtcattaaa acagaagcaa attaagacag gtttgactgc agtggtgtct ggtatccatg |
| 4681 | ttttatttct gggcacaagc tagtttttat gttgatacgt tcctgaacat attatcttgt |
| 4741 | tggacatctt ttctcttgtg ttttgtttga atgtgcaata gtttataggc cacaaataag |
| 4801 | ctttcttgta agctctcttc ctaacagggc acatattctt ccataatata aacacttttc |
| 4861 | tgccccatct cccatacttt tgaaggtcag ttctatgaca gtgaattttg cacaggagaa |
| 4921 | gcagctacct gatttcttac tttctctctc cttatcatgg agaatacaga aacattgtct |
| 4981 | gaaagggctc taaagaagga actaccaaaa cctgacttga aatgccattt cttttaacct |
| 5041 | tccaaatcct aaatgtttcc ttcaaggcat cttaataaac ttatttgctt ctggttttgg |
| 5101 | gagttcataa gagagaatag aacaaaatac aggacatcaa atattagcca tttcccattt |
| 5161 | tattttattt ttctatgtag gttcatgttc catgttcatt tatttaagaa atacattttt |
| 5221 | attggtaagc ttatagagct acacttatgg aatttttaag taggtaaata aatggttaag |
| 5281 | acaaaatagt gttatagcct tcattctctg aataggccat ctttgactca taaaattacc |
| 5341 | cttactgttt attataactt cagaagtaat ttatagttct gaacctatag tatcttttac |
| 5401 | cctgttccca agcaaagact ggtgacttta tctgaaaatg attcctcttc ccatgaccta |
| 5461 | aaacactgtg aggaaaaatc attcaagtgg catgccaagt ccctatgaag gaagggctgc |
| 5521 | tatcaaacct accttttttg agcaaactga gactaaactt ctctcttttc aaaattgtgt |
| 5581 | tatcttcctt aatcctattt tcataatttt tccttttgcc agtttttcac attatctttg |
| 5641 | atatgtgagc aacatttatt atttacatta gagtatacct tttagtaata aaatgacttg |
| 5701 | aaatcatatt atttttaaaa gccctttgct tctttcatta cttataatct cctctaaaac |
| 5761 | aacctctgca tgtttttttt aaataaagca ctttctgtca aataatggac ttttttctaa |
| 5821 | acagaaatta ttttctatta atttgcaaac tgatgatttc actttttttt actttttttt |
| 5881 | cgattatcag agtacttagt aaatgttata tagtttagtt ctaagatagt tccagggatt |
| 5941 | aaaaggttaa gaggaaaaca caaatcacca aatttctgat ttatgttttt atctcctgaa |
| 6001 | caatattttc tcactcatat tcctctatct tatcacttag ctaaagacag cctaaatttc |
| 6061 | ccaattttct gtccaaaata tttgtgattt acttgtatat aagccttctc atttgccatg |
| 6121 | tgctgtgatc ttacaagtta gatgttacta tacctaccat ttattcagct ggattgctga |
| 6181 | acacagttct ggattcatag aattaagaat atcttgttag tgcccaggat ttccacgttt |
| 6241 | tgtgttttat tggccctttt ctttattcag ccccttaatc tattttcagt cttctggcac |
| 6301 | gtaatttttt tcacagttat tattcttcta ttaacaaatt atttttatct ttcctagtac |
| 6361 | attttcactt agttctcttg cccttaaata tctttcacat gcattttagg atattctttt |
| 6421 | caaatatttg taggacaact ttgaatcaaa ataaattatg ttccttctcc aatttgaagc |
| 6481 | attgaggata aatgaccatt tgaggtctaa ctgatctttt cctgccagaa gagttatctt |
| 6541 | acgttctgct atatttgtat ttgggccagt tgattgtagg ttgtccaaca ttttttaata |
| 6601 | ttgggaaaat tatgataaaa tgctttaaaa attaatatgc cagattaaaa taactgaata |
| 6661 | gtttactatt tcattcaagc atgtttaaaa caaataattt cctttcacca gtttttctta |
| 6721 | gtaaactcct gaaaaagtag gaaaggtgga aagtatatat catttttata aattttaaat |
| 6781 | tgtacatcag acttttaaaa tctgtaatat acaagcaagc aaaattattt taaatgactt |
| 6841 | aattgtatgc taatactcat ctgataataa atgcttctta aagttgacat ttaactgcta |
| 6901 | tcacaaagtt ttatatgtag aaaagtgggg tccttttgaa taaaagatca ttcaactaaa |
| 6961 | aatattaaaa tttatttcac tggatggtaa tgtaacctta aaagcatcat aataggtaaa |
| 7021 | gtctaatatt agttccctta acaaaatcct aactgtatac cagaattagg tcactgaaag |
| 7081 | aacttgattt gaattacgtt tagacaaaaa tgatttaatt gtaaattctt aaaactttct |
| 7141 | aaatgcataa ttggcaaaaa aaaaaaccca ctgttaccag tgtaggaagt tacaagaagg |
| 7201 | cacatactga atgctgaagt atacatatgc tatttctctt aaacctcaga gcaaccatat |
| 7261 | gagcattgta attaatattc ccattttaca gatgaggaaa ctgaagctaa gagaagctaa |
| 7321 | gtaatatgcc caaggtccac atctagtaac agacaaagct gggatttcag tctatgtctg |
| 7381 | cctctctcca catctctttc atccatacca cactgcctac atgccatatg acaggatgtg |
| 7441 | taatgggcta acgtttattt aaaaagttca ggccaggtgc agtggcttat gcctataatc |
| 7501 | cccgcacttt gggaggccga gccgggtgga tcatgaggtc agacgttcaa caccagcctg |
| 7561 | gccaagatgg tgaaaccccg tctctattaa aaatattttt taaaaattaa ctgagcgtgg |
| 7621 | tggtgggcgc ctgtagtccc agctactcag gaggctaagg caggagaatc tcttgaaccc |
| 7681 | gagaggtgga ggttgcagtg agctgagatc gcgccactgc actgcagcct aggcgacaga |
| 7741 | gcaagactgc ctcaaaaaaa aaacataaaa attcagtcac tatactctgg cacaattttc |
| 7801 | atttgtatat gagccaaacc acatacctta atgatttggg aagttaggca aatatgagat |
| 7861 | atgtagacat atctatgctg attgttgctt gagaaataat tactaattct agacaaaact |
| 7921 | caatcctgta tcttccatca tgaatcttaa aatcatttca cttcactcct aacatttctt |
| 7981 | acattgcaga actaatggta aagtaaattt tacggaaaaa gttaagatag ctttgggaac |
| 8041 | agagaccttt ccctaaattg attccatagc agatttgggg gaattaacaa agaatttcag |
| 8101 | tctcatcaat cctttgaatc catcttcaaa acttctgctt ttaataactt tagaaaattt |
| 8161 | actaatctat agaactaatt gagtaggata taggaaggat acaaggatat aatgtccttt |
| 8221 | ttataaaagt ttagtatagc ttctttacat gtatccactt gttccagaaa atgtgcattg |
| 8281 | gttctgaatg tgaaaatatt taaagagaga aaggaacact caagtaagtg tgggcttcag |
| 8341 | tgggaattat cacaaaacat tggcaagtat ttttatttaa attattttca aatttgactt |
| 8401 | ctacagccaa gtggaattgg taggctgtag ctgttacact gaaatttcta gtctttgtaa |
| 8461 | gtgcctcctg aaagtcattt aaaatggaaa aatatttcaa tgagcttttc cttttttcat |
| 8521 | atttatggac atgaatattt tattggagat cattaactcc tagaatttga gattatattt |
| 8581 | ccatacaaca ttttataaag ttatgttgaa cttactacct gttatgtgca ggttattatg |
| 8641 | taactattca cagattgctt catatattgc tttatcttcc catctaactt cttaaagtta |
| 8701 | aaatccggac acacatgttg attatctaga ccagtcattc tggaaattgt aacactccca |
| 8761 | cataaacccc aggagacttt ttcagaatgc aatgtttcta aatgtactgt tactggcagt |
| 8821 | ttactctcca gcatataagg ttgcatttta acttttagat tatgaactgt gcaaacttta |
| 8881 | cccaaaacta tcttgcatga ttccctccta aatatattcc ttgattaagt aaatctggca |
| 8941 | aatcactgtt tgagctagtt acataaaatt tgttatcaag agaaggcttt tctacaagtt |
| 9001 | tccagattaa cataaagaaa agagggaatc acagggcatt taagtgcacc ttcccattac |
| 9061 | tttccttaaa tcacctcata gttaggctgg gcgcggtggc tcacgcctgt aatcccagca |
| 9121 | ctttgggagg ccgagacggt ggatcacgag gtcaggggac tgagatcatc ctggctaaca |
| 9181 | cgatgaaacc tcatctctac taaaaataca aataattagc caggcatggt ggcacgcgcc |
| 9241 | tgtagtccca gctactcggg aggcagagac aggagaatcg tttgcacccg ggaggcggag |
| 9301 | gttgttgcag tgagccgaga tcgcgccaat gcactccagc ctgggctaca gagtgagact |
| 9361 | ccatctcaaa aaaaaacaaa aaaaatcacc tcatagttta tgtctgactt actccaaacc |
| 9421 | tcagttatct gatttgtagt ctgtgtcagg aaagttttct tcatatctat tctgtctccc |
| 9481 | tcctctttga ttttaaattt ttttctttta cccagtagga caaaaaagag cagttggtca |
| 9541 | tcatccccaa tattcttagt cttcagtatg cttcaggcct ctcaatgaac acttaagtct |
| 9601 | caattcttca gacaaaattg cttaagctct tctcctcagt cctattttaa tgactttata |
| 9661 | tttaagaata tagaattata ttttctttta tattcaaatt cattactcca gttaagtaat |
| 9721 | agtttgatag tttgatagaa tcgagagtta agatgtttct atttgaaagt ggattcaacc |
| 9781 | atcagaccac cagcaaatcg gcacttaatt tttgtgttat ctaacatttt ctattgtgga |
| 9841 | attttatgat tttatattct cattagttat aactaaaaag ccatgcacac agaattgtat |
| 9901 | atcattttgc cattaaaatt ttttaacata ttgcagcaag cttagtttta tgattgagcc |
| 9961 | acaacctttt acatattttt tgtatgaaat attaaacact aaatgcaaga ttaactttca |
| 10021 | aaagcaaacc ctacattaat caggtattat ctatggacat ttttgtagac cacttttgaa |
| 10081 | atacttatta ttttgcaaca tagactggac tatacaactt tcatttaact tttaggtgac |
| 10141 | tgatttaagt tgagtgtgca tatagagaaa aacctagaaa tttatctcat ggcagataca |
| 10201 | tttgaaagta cttcagaaga atttatgctg tatattaaaa ctaggctcaa aataaatcta |
| 10261 | tcgtatcttt aaaagtccaa ttctgttatt actgtgatgt ttgtagtgtt actattaaac |
| 10321 | attgtgaaca tacacatttt taaaacaact tgaaacccat tttaaaatct gggtaagaga |
| 10381 | gaaggaatct tcagaacaaa atcacatcat tagggtgtcc agtttatgat tgaattttta |
| 10441 | agcaaattac tgtatttgaa actacaactt gatttggttt tcagttttaa aaggcaacat |
| 10501 | gtgggtttta tccattttat ttataccttt agatttcaga aacatcttca tgttttagat |
| 10561 | gcattctaca gacatcatgt tacttaaaaa ctcagggccc ctttcatccc tttgtacact |
| 10621 | gaaaaagttc aattgttagc aagtaagcaa ttagatccag ttgaatattt aaagtgtttg |
| 10681 | ttgcacagtt catttaatgt ttcatcttat ttgacttttt cacatagata taatatcaga |
| 10741 | tttcattaat tataaaaagt tgcccagttc tgtaattact gaacagaggg aatgactcaa |
| 10801 | ctaattggct acatgttgca acaaatttag gcctttagag ttgaagcact gacttaaaac |
| 10861 | gacttacatt tctgttcttt ggtcaaatga ccatacatga tatgggacaa attgtttcat |
| 10921 | tttgtttgtt ttttaataag ggaacttggt aaagtagttc ctgtcagata ggattttctc |
| 10981 | aagagacaat ttaacgttat aaagccttct aaaagtgaac taaatatttt ataactttag |
| 11041 | taatagcttg gatggttttg agaaaataac ctgtatttat cacattgtca aacagaattt |
| 11101 | ttctttgaat cagacaagtt caagctctaa attgatgtgc tatatactta aaatcctagg |
| 11161 | aagttatctg taaccagtct cttgtctcag gctcttcacc ttgttaccaa tcctcgtaag |
| 11221 | tatgtaaagg aaacatattt ttaaagaagc ttaacagtaa gaaaaaatta ctaaaagatg |
| 11281 | caattcaaag ataggtccca gtttaacact gaattgcttg acttctgtgg cttttctttt |
| 11341 | tctggccaca tttatttatt taagcaattt ttgtatgcct tgttatttca tttccataga |
| 11401 | gattatattg tatcagtgtt tatgtaagct ggaatcatcc tcagtttttt gctgataatt |
| 11461 | tttcaaataa agatacatgg ataattgtaa aatacactaa ctcttagggt gttgtagtag |
| 11521 | ctgaaacatg gagatgcgta gctgtcatgc tttttctgaa tggacaggag aaacataagc |
| 11581 | tacggagtat tcacttctga ggatgctttt ccggaaaaag aaaggctaga aaatactcgc |
| 11641 | acttcctcag aaccctcttt cttgttaacg ggtatctttt gttggtgtgt tttgctctta |
| 11701 | cattacagat agactatcat atatgacttt atgaataatt tcagttattt tgcttttgta |
| 11761 | taagctgtct gaagccttgc tatgctgtat aagttgtgtt tgatggatca gtgtgagtat |
| 11821 | aaaataaagc aaatcacttt tcttttgtat tatctatgga tgccactatg aaagctgaca |
| 11881 | ttaagccact aaagagtttt ctatgaataa gtgtaagtaa atgctttgat atatataaac |
| 11941 | ctaaataaaa agattgtatt gatacagaga cattggagaa ggagatttta aggcagttct |
| 12001 | ttaggtttaa aaaggcttgc tgtaaaatgg tgcgttattc cgtttattaa agatcatatt |
| 12061 | aatgacaa |

What is claimed is:

1. A method of treating a subject having Alzheimer's Disease (AD), the method comprising administering to the subject a therapeutically effective amount of an agent that inhibits signaling by BMPR-1A, BMPR-2, or both BMPR-1A and BMPR-2.

2. A method of increasing the rate of self-renewal of a stem cell, the method comprising contacting the stem cell with an agent that inhibits signaling by BMPR-1A, BMPR-2, or both BMPR-1A and BMPR-2.

3. The method of claim 1, which is a method of increasing the rate of neural stem cell self-renewal in the subject.

4. The method of claim 1, wherein the agent:
(a) inhibits expression of a BMPR-1A mRNA or protein;
(b) binds a BMPR-1A protein; and/or
(c) inhibits interaction between a BMP protein and a BMPR-1A.

5. The method of claim 1, wherein the agent:
(a) inhibits expression of a BMPR-2 mRNA or protein;
(b) binds a BMPR-2 protein; and/or
(c) inhibits interaction between a BMP protein and a BMPR-2.

6. The method of claim 1, wherein the agent is a nucleic acid.

7. The method of claim 6, wherein the agent is a small interfering RNA (siRNA) or a short hairpin RNA (shRNA) that targets BMPR-1A, BMPR-2A, or both BMPR-1A and BMPR-2.

8. The method of claim 6, wherein the agent is an antisense oligonucleotide (ASO) that targets BMPR-1A, BMPR-2A, or both BMPR-1A and BMPR-2.

9. The method of claim 6, wherein the agent is a guide RNA (gRNA).

10. The method of claim 1, wherein the agent is a protein or an aptamer.

11. The method of claim 10, wherein the agent is an antibody.

12. The method of claim 11, wherein the agent is a blocking or neutralizing antibody that binds specifically to BMPR-1A, BMPR-2A, or both BMPR-1A and BMPR-2.

13. The method of claim 2, wherein the stem cell is a neural stem cell or a neural progenitor cell.

14. A method of preparing a medicament for treating Alzheimer's Disease (AD) or increasing neural stem cell self-renewal in a subject in need thereof, the method comprising:
identifying a compound that is effective as an agent to inhibit signaling by BMPR-1A, BMPR-2, or both BMPR-1A and BMPR-2 on neural stem cells, and
compounding a therapeutically effective amount of the compound with a pharmaceutically acceptable excipient so as to produce the medicament.

15. The method of claim 14, wherein the agent:
(a) inhibits expression of a BMPR-1A mRNA or protein;
(b) binds a BMPR-1A protein; and/or
(c) inhibits interaction between a BMP protein and a BMPR-1A.

16. The method of claim 14, wherein the agent:
(a) inhibits expression of a BMPR-2 mRNA or protein;
(b) binds a BMPR-2 protein; and/or
(c) inhibits interaction between a BMP protein and a BMPR-2.

17. The method of claim 14, wherein the agent is a nucleic acid selected from a small interfering RNA (siRNA), a short hairpin RNA (shRNA), an antisense oligonucleotide (ASO) and a guide RNA (gRNA), wherein the nucleic acid targets BMPR-1A, BMPR-2A, or both BMPR-1A and BMPR-2.

18. The method of claim 14, wherein the agent is a blocking or neutralizing antibody that binds specifically to BMPR-1A, BMPR-2A, or both BMPR-1A and BMPR-2.

19. A unit dose of a medicament prepared according to the method of claim 14, wherein formulation of the medicament and the amount of the agent contained in the unit dose are selected such that the unit dose is effective in treating Alzheimer's Disease (AD) in a subject in need thereof.

20. A unit dose of a medicament prepared according to the method of claim 14, wherein formulation of the medicament and the amount of the agent contained in the unit dose are selected such that the unit dose is effective in increasing the rate of neural stem cell self-renewal in a subject in need thereof.
